(19) United States
(12) Patent Application Publication
(10) Pub. No.: US 2025/0319296 A1
(43) Pub. Date: Oct. 16, 2025

(54) ULTRASOUND-MEDIATED GENE AND DRUG DELIVERY

(71) Applicant: Seattle Children’s Hospital d/b/a Seattle Children’s Research Institute, Seattle, WA (US)

(72) Inventors: Carol Hsing Miao, Seattle, WA (US); Feng Zhang, Seattle, WA (US)

(73) Assignee: Seattle Children’s Hospital d/b/a Seattle Children’s Research Institute, Seattle, WA (US)

(21) Appl. No.: 19/253,010

(22) Filed: Jun. 27, 2025

Related U.S. Application Data
(63) Continuation of application No. 17/051,141, filed on Oct. 27, 2020.
(60) Provisional application No. 62/663,939, filed on Apr. 27, 2018.

(51) Int. Cl. A61M 37/00; A61B 8/00; A61K 48/00; A61M 25/10; A61N 7/00

(52) U.S. Cl. CPC A61M 37/0092; A61B 8/481; A61K 48/0075; A61M 2025/1052; A61N 2007/0039

(57) ABSTRACT

Transcutaneous, ultrasound-mediated methods for administering compound(s) to subject tissue(s) are provided. Examples involve positioning an occluding device in a vessel such that the blockage is adjacent to target tissue; engaging the device to occlude outflow from a region adjacent to the tissue; administering compound(s) to the vessel such that it is substantially retained adjacent to the target tissue; determining the location of the compound and/or a detectable adjunct compound optionally administered with the compound, using diagnostic ultrasound, radiography, or fluorography; administering therapeutic ultrasound energy transcutaneously to mediate delivery of the compound across the vessel wall and into adjacent target tissue.

### CROSS-REFERENCE TO RELATED APPLICATIONS

This application is a continuation application which claims priority to U.S. patent application Ser. No. 17/051,141, filed on Oct. 27, 2020, which is a U.S. National Phase Patent Application based on International Patent Application No. PCT/US2019/029492 filed on Apr. 26, 2019, which claims priority to U.S. Provisional Application No. 62/663,939 filed on Apr. 27, 2018, the contents of each of which are incorporated herein by reference in their entirety as if fully set forth herein.

### STATEMENT REGARDING FEDERALLY SPONSORED RESEARCH OR DEVELOPMENT

This invention was made with government support under HL128139 awarded by the National Institutes of Health. The government has certain rights in the invention.

### REFERENCE TO SEQUENCE LISTING

The Sequence Listing associated with this application is provided in xml format in lieu of a paper copy, and is hereby incorporated by reference into the specification. The name of the text file containing the Sequence Listing is 3G27015.XML. The text is 4,096 bytes, was created on Jun. 20, 2025, and is being submitted electronically via Patent Center.

### FIELD OF THE DISCLOSURE

The present disclosure provides systems and methods for administering a compound to a targeted tissue. More particularly, it relates to using ultrasound to deliver a compound (such as a nucleic acid molecule or a drug) to tissue(s) within a subject.

### BACKGROUND OF THE DISCLOSURE

Non-viral gene therapy confers appreciable benefits over viral methods, including lower risk of immunopathogenicity, greater flexibility in vector construction, and better spatial and temporal control. Delivery of plasmid DNA (pDNA) is particularly attractive as manipulation of (and possible damage to) the host genome can be avoided and the vector can more easily be engineered for episomal persistence and long-term promoter activation.

Ultrasound (US)-mediated gene delivery (UMGD) has long been recognized as a potential method to perform minimally invasive, non-viral gene transfer of pDNA. Effective UMGD relies on the presence of microbubbles (MBs), which have been demonstrated to significantly enhance gene transfer efficiency, resulting in increased transgene expression. Under appropriate acoustic pressures and applied frequencies, spontaneous formation of gas cavities, termed cavitation, may occur. MBs serve as cavitation nuclei and can oscillate radially and collapse when exposed to a driving pressure field. Although the precise mechanism is not entirely known, MB cavitation and/or destruction during therapeutic sonication is shown to facilitate transient pore formation along the cell membrane (De Cock et al., J Control Release 197:20-28, 2015; Hallow et al., Ultrasound Med Biol. 32:1111-1122, 2006). Acoustic cavitation of MBs may also increase permeability of endogenous barriers such as the cell membrane or vessel wall to allow normally impermeable materials (e.g., drugs or macromolecules) to cross via diffusion.

Other non-viral gene therapies include systemic exposure to lipid nanoparticles carrying genetic material or direct injection of gene vectors to tissue-specific sites (e.g., intraparenchymal or intramuscular). However, use of lipid or polymer encased pDNA may be hindered by difficulty in packaging, expelling genetic load, and avoiding cytoplasmic degradation. In addition, direct injection to tissue-specific sites faces the challenge of traversing the plasma membrane of target cells. Alternatively, systemic administration of genetic vectors is also challenged by the multiple barriers hindering entry of pDNA into cells. The vectors must first cross the endothelium, the basement membrane, and smooth muscle layer before overcoming the outer cell membrane of the target cells and finally the nuclear membrane for efficient gene expression. UMGD may potentially overcome some or all of these barriers to significantly improve gene transfer efficiency targeting specific tissue/cells. Incorporation of UMGD methods with non-viral gene therapy is motivated by the aim of treating genetic diseases, such as hemophilia, that is safe with comparable efficiency to viral gene transfer methods.

The liver is an ideal target for gene therapy in hemophilia A patients, as it is a predominant site of factor VIII production, and where deficiency of the protein is responsible for the hemophiliac phenotype. It has previously been shown that UMGD can significantly enhance gene transfer in livers of both small and larger animal models (Noble et al., Mol Ther. 21:1687-1694, 2013; Song et al., Mol Pharm. 9:2187-2196, 2012; Song et al., Gene Ther., 18:1006-1014, 2011; Shen et al., Gene Ther. 14:1147-1155, 2008). In mouse models, MBs and pDNA encoding a reporter gene were co-administered by injection into the portal vein (PV) while US was simultaneously applied to the liver lobes. This treatment protocol had to be revised in rats to accommodate the larger liver lobes, by injecting the MBs into individual liver lobes through a PV branch. Similar to the rat studies, a nearly 100-fold increase in average transgene expression was achieved relative to sham, when the single lobe injection strategy was translated to a dog models. In agreement with small animal studies, the dog model indicated that a peak negative pressure (PNP) of about 2.7 MPa is required for effective gene transfection with minimal liver tissue damage. However, the current surgical procedure requires opening the cavity of the animal to treat the surface of the liver. It would be highly beneficial to develop a minimally invasive surgical procedure that could still facilitate effective UMGD.

A clinically applicable procedure would involve transcutaneous UMGD. Given the 2.7 MPa threshold necessary for effective gene transfer into the liver cells however, acoustic pressures beyond the capabilities of art-recognized piezo-material would be required due to the loss of acoustic energy passing through several tissue layers.

Thus, there remains need for additional methods of US compound delivery that can deliver nucleic acids or other therapeutic compounds into targeted tissues in a subject, as well as systems and devices that can be used with and facilitate such methods.

### SUMMARY OF THE DISCLOSURE

The current disclosure describes compositions and methods that enable targeted, ultrasound-mediated gene and drug delivery in large animals; the delivery in some embodiments is enhanced by targeted destruction of microbubbles. These methods are optionally enhanced further by using a percutaneous surgical procedure to access a target tissue site, and facilitated by a combination of fluoroscopy and diagnostic ultrasound imaging techniques. Therapeutic ultrasound is then used to insonate microbubbles in the presence of the therapeutic compound (typified herein by naked plasmid DNA), thereby transferring the compound to a large volume of tissue transcutaneously.

In certain embodiments, administration of the treatment is performed with novel ultrasound transducers, systems, and parameter settings. For example, a signal generator/amplifier has been designed for greater electrical power output to a new transducer configuration that can withstand the input without the piezo-material deteriorating. With this modified ultrasound technology, methods are enabled that apply high peak pressures with a low duty cycle while maintaining high effective treatment volume and penetration at a high frequency (1 MHZ). This technology permits high-efficiency gene transfer for ultrasound-mediated deliver (UMD) and offers a novel method to treat large volumes of tissue in large animals (including humans) with minimal tissue damage. As explained below, tissue damage is minimalized through the herein-described protocols that apply relative lower pressure (such as 0.5-6 Mpa) and/or relatively longer pulse period (such as 19 microseconds to 22 milliseconds). Specific examples are provided herein.

The methods, systems, and devices described herein can be used for gene and drug delivery to tissues at a large (for instance, in subjects over 2 kilograms in mass) animals and humans. They are also useful in other applications requiring medium-to high-intensity ultrasound targeting of tissue areas. By way of example, the herein described methods, systems, and devices may be used for targeted therapeutic compound (e.g., drug) delivery to tumor sites including where the stromal or cellular environment is too dense for penetration by conventional ultrasound.

In representative transcutaneous ultrasound protocols, components may include one or more of percutaneous access to a target site, percutaneous delivery and capture of therapeutic compound(s) adjacent (near) to a tissue to be treated, fluoroscopic-assisted targeting, diagnostic ultrasound-assisted targeting, microbubble insonation, and/or application of sonic waves using ultrasound transducer(s) developed herein.

While ultrasound-mediated gene delivery (UMGD) has been accomplished using high peak negative pressures (PNPs) of 2 MPa or above, it may not be a requirement for microbubble (MB) cavitation. Thus, lower-pressure conditions close to the MB inertial cavitation threshold were investigated, and additional efforts were directed towards increasing gene transfer efficiency and reducing associated cell damage. Longer pulse duration conditions yielded significant increase in transgene expression relative to sham with local maxima between 20J and 100J energy curves. A local maxima between 1J and 10J energy curves was observed in treated mice. Of these, several low pressure conditions showed a decrease in ALT and AST levels while maintaining better or comparable expression to the positive control, indicating a clear benefit to allow for effective transfection with minimized tissue damage versus the high-intensity control. The data presented here indicate that it is possible to eliminate the requirement of high PNPs by prolonging pulse durations for effective UMGD in vitro and in vivo, circumventing the peak power density limitations imposed by piezo-materials used in US transducers. Overall, these results demonstrate the advancement of UMGD technology for achieving efficient gene transfer and potential scalability to larger animal models and human application.

Thus, transcutaneous, ultrasound-mediated delivery methods for administering a therapeutic compound to a target tissue in a subject are provided. Examples of such methods involve positioning a positionable occluding device (e.g., a balloon catheter) in a blood vessel of the subject such that the resultant blockage is adjacent to the target tissue; engaging the occluding device to occlude outflow from a region adjacent to the target tissue; administering the therapeutic compound to the vessel of the subject such that it is substantially retained adjacent to the target tissue by the occluding device; determining the location of the therapeutic compound and/or a detectable adjunct compound (such as an ultrasound contrast agent, radioisotope, or the like) administered with the therapeutic compound using at least one of diagnostic ultrasound, radiography, or fluorography; administering therapeutic ultrasound energy (sonication) transcutaneously, such that the energy mediates delivery of the therapeutic compound across the vessel wall and into the adjacent target tissue. Specific methods described herein employ a therapeutic US device with at least some of the following features: small form factor, ergonomic to subject, high effective treatment volume, good penetration, operation at 1 MHZ, and high peak pressures with low duty cycle. Specifically, such a tUS is used to insonicate microbubbles transcutaneously, for instance where such microbubbles have been localized in the subject and that localization confirmed using one more of diagnostic ultrasound, fluorography, or radiography.

### BRIEF DESCRIPTION OF THE FIGURES

At least one of the drawings submitted herewith is better understood in color. Applicants consider the color versions of the drawing(s) as part of the original submission and reserve the right to present the color images of the drawings in later proceedings.

FIGS. 1A-1C: Overview of in vitro and in vivo experimental apparatus and setup. (FIG. 1A) Block diagram of signal generation and real-time monitoring. (FIG. 1B) Schematic representation of in vitro experiments done in an anechoic water bath. Cell/pDNA/MB suspension is treated 8 mm from the face of the transducer where maximal PNP occurs. (FIG. 1C) Representation of in vivo sonoporation experiments performed in livers of mice. A pDNA/MB solution is injected via the portal vein and the liver exposed to US simultaneously. US treatment on surface of liver was performed 8 mm from the face of the transducer.

FIGS. 2A-2C: Transgene expression after UMGD in HEK293T cells and analysis of results grouped by treatment energy bands. (FIG. 2A) Expression and (FIG. 2B) viability results across pulse durations (18 μs-36 ms) and PNPs (0.5-2.5 MPa). Expression was measured 48 hrs post-transfection using flow cytometry and quantified as product of MFI and percentage Live/GFP+. Expression values are normalized relative to the highest performing condition by average expression. Increasing GFP expression corresponds to a shift in color shade from light to dark. Viability was measured via 7-AAD staining using flow cytometry, quantified as the ratio of 7AAD/Total Single Cells. Increasing viability corresponds to a shift in color from red to green. Each circle represents one group of cell culture experiments at a distinct condition (n=2-8 experiments/group), with color linearly related to GFP expression or viability. Equi-energy curves (1, 10, 20, 60, and 100 Joules (J)) are plotted for both (FIG. 2A) and (FIG. 2B). Distribution of experimental data provided in FIG. 8A-B. (FIG. 2C) US parameter pairings that produce treatment energies at or above 60J resulted in significantly greater overall transfection than pairings that produce treatment energies below 60J. Data is presented as pooled averages with error bars indicating standard deviation. ** P<0.005, *** P<0.0005.

FIGS. 3A-3D: Transgene expression after UMGD in C57/BL6 mice and analysis of results grouped by treatment energy bands. (FIG. 3A) Luciferase expression across 32 conditions, varying pulse durations (18 μs-36 ms) and PNPs (0.5-2.5 MPa). Liver tissue was harvested 24 hrs post-treatment, and expression was evaluated via luciferase assay of excised tissue. Results were normalized against protein content of each sample as determined by Bradford protein assay. Increasing luciferase expression corresponds to a shift in color shade from light to dark. (FIG. 3B) US parameter pairings that produce treatment energies between 1-10J resulted in significantly greater overall transfection than pairings that produce treatment energies below 1J or above 10J. Group comparison of conditions that lie below 1J or above 10J show no statistical significance. Data is presented as pooled averages with error bars indicating standard deviation. ** P<0.005, *** P<0.0005. RLU, relative light unit. (FIG. 3C) ALT levels, and (FIG. 3D) AST levels in plasma of treated mice following UMGD. Increasing levels in ALT and AST correspond to a shift in color from green to red. Plasma was isolated 24 hrs post-treatment for examining the hepatotoxicity by a colorimetric AST/ALT assay. Results were quantified using a plate-reader and calibrated to known standards for each run. Plotted lines are equi-energy curves (1, 10, 20, 60, and 100 Js). Each circle represents one group of mice at a distinct condition (n=5-10 mice/group), with color linearly related to luciferase expression. Distribution of experimental data provided in FIG. S4.

FIG. 4: pDNA stability tested by exposing pGL4.13 [sv40/luc2] to US in the presence of MBs. A pulse duration of 18 μs at 2.5 MPa was used with a treatment time of 60s. RN18 MBs were used. Following US treatment, plasmid was linearized using Bgl II. Lane 1 is a 1kbp ladder, lanes 2-4 are pGL4.13 digested by Bgl II, lanes 5-7 are undigested pGL4.13. Lanes 4 & 7 indicate that neither US nor MB exposure affected the integrity of the DNA sequence or structure.

FIG. 5: Cavitation versus US treatment energy. RN18 MBs (˜1×10⁹ MB/mL) were diluted 1:1000 in PBS and mixed gently prior to 60s of US exposure in the in vitro setup. The remaining percentage of MBs with respect to an increase in US treatment energy follows an exponential decay trend. A range of conditions (18 μs-36 ms and PNPs 0.5-2.5 MPa) were tested with n≥2 for each condition. MB destruction was quantified using gated counts of MBs from flow cytometry, relative to sham-exposed MBs. Each circle represents one group with a distinct US condition.

FIG. 6: Relation of luciferase expression to GFP expression by correlation. HEK 293T cells were transfected with either pGL4.13 or pGFP using lipofectamine. Cells were transfected with 0.5%, 1%, and 2% (v/v) pDNA under several UMGD conditions. Cells transfected with pGL4.13 were assayed using the luciferase assay kit. Luciferase expression was normalized to protein levels and presented as RLU/mg protein. Cells transfected with pGFP were analyzed using flow cytometry. GFP expression presented as the product of MFI and percent GFP+/Total Live Single Cells.

FIGS. 7A-7B: Fluorescent microscopy with associated flow analysis 48-hrs post-US transfection of HEK293T cells. All microscopy images were taken at 10× magnification. (FIG. 7A) Representative fluorescent microscopy images depicting results of UMGD of pGFP before flow analysis. Scale bar=200 μm. (FIG. 7B) Representative flow analysis shows division of cell population into quartiles with 7AAD (PE)- and GFP (FITC)-fluorescence. Increasing 7AAD signal represents decreased viability. Gating strategy is shown on non-treated HEK293T cells (neg ctrl). MFI=median fluorescence intensity.

FIGS. 8A-8B: Distribution of HEK293T UMGD data. (FIG. 8A) Normalized GFP expression organized by peak negative pressure (PNP) and applied pulse duration. For each pair of PNP and pulse duration settings, a solid line and shaded region are used to denote the average normalized GFP value and values within +1 standard deviation of the average, respectively. (FIG. 8B) Cell viability (as a percentage) organized by peak negative pressure (PNP) and applied pulse duration. For each pair of PNP and pulse duration settings, a solid line and shaded region are used to denote the average value and the 95% confidence interval of the viability, respectively.

FIGS. 9A-9C: Distribution of murine UMGD data. (FIG. 9A) RLU/mg protein values organized by peak negative pressure (PNP) and applied pulse duration. For each pair of PNP and pulse duration settings, a solid line and shaded region are used to denote the average RLU/mg protein and values within +1 standard deviation of the average, respectively. FIG. 9B ALT concentration in units per liter organized by peak negative pressure (PNP) and applied pulse duration. For each pair of PNP and pulse duration settings, a solid line and shaded region are used to denote the average value and values within +1 standard deviation of the average, respectively. (FIG. 9C) AST concentration in units per liter organized by peak negative pressure (PNP) and applied pulse duration. For each pair of PNP and pulse duration settings, a solid line and shaded region are used to denote the average value and values within +1 standard deviation of the average, respectively.

FIGS. 10A-10C: Comparison of US parameter pairings resulting in best overall transfection to the positive control (2.5 MPa, 18 μs). (FIG. 10A) Several US parameter pairings were selected that had comparable expression to the positive control and showed no significant difference. (FIG. 10B) ALT values were compared using the same US parameter pairings to the positive control. (1.1 MPa, 400 μs) showed a significant decrease in ALT levels after treatment compared to (2.5 MPa, 18 μs). Some conditions show a significant increase in ALT levels. (FIG. 10C) AST values were compared using the same US parameter pairings to the positive control. Two conditions show a significant increase in AST levels compared to (2.5 MPa, 18 μs). Applied pressures above 1.1 MPa and pulse durations greater than 22 ms were therefore avoided. Error bars indicate standard deviation. * P<0.05, ** P<0.005. RLU, relative light unit.

FIG. 11: Ultrasound-mediated gene delivery (UMGD).

FIGS. 12A-12B: Improvements in UMGD-high peak negative pressure (PNP), focused transducer enhanced gene transfer. FIG. 12A is a pressure profile map of the beam pattern for one of the developed focused transducers. FIG. 12B is a plot of luciferase expression, reported as relative light units/mg protein on the y-axis, with respect to increasing PNP settings. Each dot depicts one liver tissue section.

FIGS. 13A-13B: Lower peak power density still enhanced gene transfer. FIG. 13A is a plot of average luciferase expression in individual pigs, represented as dots, in different US treatment groups. FIG. 13B shows the fold-enhancement of gene expression relative to sham.

FIG. 14: Illustration of clinically relevant, lower risk, multimodal approach to targeted UMGD employing a balloon catheter coupled with sonography and fluoroscopy (right panel), compared to current laparotomy-based direct ultrasound application (left panel).

FIGS. 15A-15B: Therapeutic US (tUS) localized by guided-US and fluoroscopy. In FIG. 15A, the main hepatic branches in a pig were mapped to be used as a reference for catheter insertions in later experiments. Several catheter exchanges were first performed, to access the desired hepatic vein branch and deploy a balloon catheter. The balloon is inflated to occlude blood outflow and retain the pDNA/MB solution. A solution of MBs is then infused after catheter placement, and visualized using diagnostic ultrasound before US treatment. This helps to determine the location and angle the therapeutic transducer should be placed. FIG. 15B, Top (left lobe) and bottom (right middle liver lobe) panels: left, fluoroscopy picture of catheter insertion into the liver lobe; middle, ultrasound imaging of contrast agent on the specific liver lobe before therapeutic ultrasound treatment; right, ultrasound imaging of contrast agent on the specific liver lobe after therapeutic ultrasound treatment.

FIGS. 16A-16B: Transducer (XDR) modification for UMGD, showing the beam pattern from H105 (52 mm diameter) and H114 (90×4 mm) transducers (Sonic Concepts, Inc.). Pressure profile maps of H105 and H114 show differences in beam pattern and transaxial uniformity. H105 is a planar, disc transducer shown in (FIG. 16A) with pressure profiles at depths of 25 mm (left) and 45 mm (right). Due to its unfocused nature, the relative pressure distribution is nearly constant with respect to depth when comparing the two slices. H114 is a geometrically focused transducer shown in (FIG. 16B) with pressure profiles at depths of 25 mm (top) and 45 mm (bottom). Moving from 25 mm to 45 mm, the beam pattern narrows significantly, and this focal convergence results in an increased maximal relative pressure.

FIG. 17: Table of treatment protocol conditions used with H105 and H114 transducers.

FIGS. 18A-18B: UMGD efficiency maintained via transcutaneous application. Using either the H105 or H114 transducer achieved significant gene transfer compared to the control group. Interesting, with increasing pulse duration and decreasing PNP, w even greater expression levels were obtained-especially with H114. The highest expressing points were greater than 105 RLU/mg protein using H105 and H114 at a pulse duration setting of 2 ms. This may be due to less overall tissue damage and cell death from using lower US PNPs. In general, luciferase gene transfer was enhanced by at least 500-fold using all US treatment groups.

FIG. 19: Safety profile and gene transfer spatial distribution, using H105 and H114 transducers.

FIG. 20: XDR modification furthers UMGD success, both with XDR106 5 element (10×80 mm) and XDR106 10 element (40×80 mm) custom transducers (Sonic Concepts, Inc.).

FIG. 21A-21C. FIG. 21A. The hepatic vasculature was mapped by occluding the inferior vena cava above and below the hepatic venous system, and used as a reference image guide for catheter insertion in later experiments. FIG. 21B-21C Catheter placement is consistently placed and confirmed via fluoroscopy. Representative fluoroscopy images accessing the left and right hepatic lobes are depicted in two separate pigs. The left and right lateral hepatic lobes were easily and consistently accessed without perforation of the vasculature and tissue. Contrast agent is dispensed through the catheter within the vasculature after outflow is occluded from the expanded balloon. Two lobes are accessed during each surgery. Each lobe is treated with either a different therapeutic transducer or US protocol.

FIGS. 22A-22C. Fluoroscopy-guided transhepatic venous infusion of plasmid NDA with MBs in the left lateral lob (LLL) and right medial lobe (RML). (FIG. 22A) Angiography of the left and middle hepatic vein confirmed the location of the balloon catheter and where the microbubbles/DNA infused through the same balloon catheter would distribute. (FIG. 22B-22C) Representative ultrasonography of MBs present in the hepatic vasculature before tUS treatment (FIG. 22B) and after treatment (FIG. 22C), showing the distribution of MBs in the liver lobe corresponding to the distribution of x-ray contrast (Visipauqe) (FIG. 22B) and the significant decrease in the vasculature post US treatment (FIG. 22C).

FIGS. 23A-23B. Strategy to determine transducer positioning prior to tUS. A solution of MBs are first injected through the secured balloon catheter and MB distribution is first determined via diagnostic US imaging (FIG. 23A). Depth and spread of MB distribution is visualized and determined to be appropriate for tUS treatment. Angle and placement of diagnostic US probe is noted and replaced with the tUS transducer probe (H114 depicted) (FIG. 23B). Same approximate angle and placement of tUS transducer probe is aligned prior to start of US treatment.

FIGS. 24A-24B. Pressure profile maps of H105 and H114 show differences in beam pattern and transaxial uniformity. H105 is a planar disc transducer shown in (FIG. 24A) with pressure profiles at depts of 25 mM (left) and 45 mm (right). Due to its unfocused nature, the representative pressure distribution is nearly constant with respect to depth when comparing the two slices. H114 is a geometrically focused transducer shown in (FIG. 24B) with pressure profiles at depths of 25 mm (top) and 45 mm (bottom). Moving from 25 mm to 45 mm, the beam patter narrows significantly, and this focal convergence results in an increased maximal relative pressure.

FIGS. 25A-25B. Resulting luciferase gene expression represented as activity levels for H105 and H114 μsing different US protocols. Data from multiple pigs treated with a particular US protocol were pooled for analysis. (FIG. 25A) All US protocol groups listed in Table 3 are compared to the control group (no MBs+no US treatment). The following lists the exact number of pigs and tissue sections used for analysis: (Control), N=3, n=26; (H105: 19 μs,2.2 MPa), N=1, n=8; (H105: 200 μs, 1.2 MPa), N=3, n=39; (H105: 2 ms, 0.8 MPa), N=2, n=34; (H114: 19 μs, 5.1 MPa), N=2, n=16; (H114: 200 μs,2.5 MPa), N=4, n=55; (H114: 2 ms, 1.7 MPa), N=2, n=47. All US protocols using either transducer shows significant increase in gene expression relative to control (p ****<0.0001). No significant difference is observed comparing across US protocols using either H105 or H114. Data is shown with upper and lower quartiles with median values. (FIG. 25B) Average gene enhancement using either H105 or H114 for all US protocols are able to achieve over 100-fold increase relative to control. Error bars represent standard deviation.

FIG. 26. Spread of pooled data from therapeutic US treatment groups and control group. Pooled data from all US protocols are represented as box and whisker plots to show the spread of data including outliers. Lines within the box represent the median of the data set with upper 75% and lower 25% quartiles to show the majority spread of data. Upper and lower lines of each box represent the maximum and minimum data point for a particular data set, respectively. ‘+’ symbols represent the mean of the data.

FIGS. 27A-27B. Representative spatial gene distribution plots depicting a correlation between catheter placement and resulting location of luciferase activity. Sectioned tissue was recorded for location on a cartesian map and assayed. Resulting luciferase activity level for assayed tissue sections were overlaid onto a mapped plot of the liver lobe. S+ Superior, I=Inferior, relative to head and tail of the pig. Spatial gene distribution maps are shown according to transducer and US protocol used; (FIG. 27A) H114 (200 μs, 2.5 MPa), (FIG. 27B) H105 (200 μs, 1.2 MPa). Corresponding fluoroscopy images are shown to the right of the spatial distribution maps. This spatial distribution qualitative analysis was performed for every liver lob assayed, for a total of 31 liver lobes.

FIGS. 28A-28D. Visualization of luciferase activity on a Cartesian coordinate system revealed correlative gene distribution to fluoroscopy imaging. Points of expression were localized within regions of catheter placement as verified by corresponding fluoroscopy images. Only treated left lateral hepatic lobes are shown. Representative spatial gene distribution maps are shown according to transducer and US protocol used: (FIG. 28A) H105 (19 μs, 2.2 MPa), (FIG. 28B) H105 (2 ms, 0.8 MPa), (FIG. 28C) H114 (19 μs, 5.1 MPa), (FIG. 28D) H114 (2 ms, 1.7 MPa).

FIG. 29A-29G. Trichome stained liver biopsies revealed variable tissue damage depending upon transducer and US protocol used. Trichome stained slides focusing on the area of maximal hepatic damage for all treatment groups including control are shown. Black arrows point to areas of pericentral hemorrhage and necrosis. The histological analysis is shown at 2.5× magnification. FIG. 29A: Control; FIG. 29B: H114 (19 μs, 4.1 MPa); FIG. 29C: H114 (200 μs, 2.5 MPa); FIG. 29D: H114 (w ms, 17. MPa); FIG. 29E: H105 (19 μs, 2.2 MPa); FIG. 29F: H105 (200 μs, 1.2 MPa); and FIG. 29G: H105 (2 ms, 0.8 MPa).

FIGS. 30A-30B. Distribution of gene expression in liver tissue following US/MB mediated gene delivery. Reporter gene plasmid pGL4 or pGFP were respectively delivered into BL6 mouse liver through portal veil for 30 sec with simultaneous 60 sec-US exposure (1 sec on 2 sec off, 1.1 MHz frequency, 20 cycle pulses, 2.7 MPa PNP, 13.9 Hz PRF). A unfocused H158 transducer was used in these experiments. (FIG. 30A) Luciferase expression levels on a 10⁷ cells basis in hepatocytes and non-parenchymal cells. On day 1 post treatment, pGL4 plasmid transferred mouse livers were perfused and isolated into two cell populations: hepatocytes and non-parenchymal cells and luciferase expression were evaluated in the 2 cell populations. (FIG. 30B) Representative GFP fluorescence staining of US/MB treated liver and plasmid only injected liver. Mouse liver transferred with pGFP was sectioned and stained to show GFP protein expression.

FIGS. 31A-31D. Gene expression and antibody production following naked gene transfer of FVIII and FVIII/N6 plasmids. hFVIII levels and inhibitory antibody formation over time in HemA mice after treatment with pBS-HCRHPI-hFVIIIA (FIGS. 31A & 31B) or pBS-HCRHPI-hFVIII/N6 (FIGS. 31C & 31D) plasmids at weeks 1,2,4 & 8. Due to the large size of FVIII, a B-domain deleted FVIII (BDD-FVIII) cDNA is usually used for developing gene therapies for HemA. BDD-FVIII consisting of a partial B-domain deletion leaving an N-terminal 226 amino acid stretch containing 6 intact putative asparagine-linked glycosylation sites (FVIII/N6) increases in vitro and in vivo secretion of FVIII by 10-15 fold. We inserted this modified FVIII/N6 cDNA into our liver-specific gene expression vector. The resulting construct, FVIII/N6 plasmid, was delivered hydrodynamically into HemA mouse liver. In control mice treated with FVIII plasmid containing the BDD-FVIII cDNA, FVIII expression levels dropped to undetectable levels 2 weeks post injection and high-titer anti-FVIII antibodies were generated in all mice. However, most mice treated with FVIII/N6 plasmid produced lower titers of inhibitory antibodies compared to the control mice. These findings suggest that a reduced dosage and duration of the immunomodulation protocol may be sufficient to modulate the anti-FVIII immune responses following gene transfer of a FVIII/N6 construct.

FIGS. 32A-32C. hFVIII activity achieved using hFVIII plasmids in HemA mice following UMGD. 50 μg hFVIII plasmid mixed with 5 Vol % MBs were injected via the portal vein of HemA mouse liver for 30 s, with simultaneous US exposure (1.1 MHZ, 2.7 MPa P-, 20 cycle pulses, 13.9 Hz PRF) for 60 s. For gene transfer of hFVIII, hFVIII gene expression in plasma was evaluated by a modified aPTT assay. (FIG. 32A) Schematics of the FVIII constructs. (FIG. 32B) hFVIII activity at day 3 and 7 post treatment using pLC-hF8/N6, (FIG. 32C) hFVIII activity at day 3 and 7 post treatment using pLC-hF8-X10

FIGS. 33A-33B. Gene therapy of hFVIII plasmid mediated by US/MB in hemophilia A mice. All mice were pretreated with IL-2/IL-2mAb complexes to modulate immune response. 100 μg hFVIII plasmid mixed with 5 Vol % NuvOx MB was injected for 30 sec with simultaneous pulse-train US exposure (1 sec on 2 sec off, 1.1 MHz frequency, 20 cycle pulses, 2.0 MPa PNP, 13.9 Hz PRF) for 60 sec. FVIII expression levels in hemophilia A mice were significantly enhanced following US/MB mediated gene transfer and immunomodulation, shown as: (FIG. 33A) Long-term FVIII activities in mice plasma assessed by a modified activated partial thromboplastin time (APTT) assay and (FIG. 33B) Inhibitory antibody against FVIII were assessed by Bethesda assay. Each symbol represents data obtained from an individual mouse.

FIG. 34 Phenotype correction of hemophilia A in HA/Balb/C mice following US/MB mediated gene transfer. Total blood loss upon tail tip clip resection as measured by released hemoglobin levels. Bleeding correction was calculated based on normal mice as positive control (100%) and untreated HA/Balb/C as negative control (0%).

FIGS. 35A-35B Evaluation of pBSHCRHP-human FVIII/N6 plasmid copy number in mouse liver. (FIG. 35A) Standard curve (equation y=−3.45x+36.254) generated with serial dilutions of the plasmid, which were assayed in duplicate by real-time qPCR. (FIG. 35B) Calculated FVIII plasmid copy number per ng DNA in the untreated mice, US treated mice on day 1, 4 and hydrodynamic injected mice on day 1 (n=3). Genomic DNA was extracted from the liver tissues and real-time PCR was performed in duplicate.

FIGS. 36A-36F Representative histological features of hemophilia mouse livers injected with plasmid and MB via portal vein and simultaneously treated with 2.0 MPa pulse-train US exposure. The serial liver sections (FIG. 36A-36F) were harvested on day 0, 1, 3, 7, 14, 28 respectively after treatment for H&E staining. Original magnification X100, X400 in inserts. Scale bar=50 μm.

FIG. 37A-37B. Luciferase gene expression following UMGD into pig livers show equivalent expression in prolonged pulse duration, lower pressure groups. All pigs were injected with 0.67 mg/kg pGL4 plasmid, 0.2 mL/kg MBs via the segmented PV branch with simultaneous exposure of the target liver lobe to therapeutic US (1.05 MHz applied frequency, 19-2100 cycle pulses, 50 Hz PRF, and 2.6-6.9 MPa PPNs). (FIG. 37A) Absolute gene expression for each group: Sham (n=2), control (n=18), ([19 μs, 6.9 MPA]; n=8), ([200 μs, 4.6 MPa] n=5), and ([2 ms, 2.6 MPa]; n=5). All three US-treated groups showed significant increase in expression compared to sham. There was no significant difference between the three US-treated groups. Each circle represents one pig. Horizontal lines indicate average value within a US pairing group. Error bars indicate standard deviation. (FIG. 37B) The average luciferase expression from each US-treated group was normalized to the average luciferase expression of the sham-treated pig to determine the overall gene enhancement. Data shows at least a 100-fold increase in expression compared to sham for all US-treated groups. * P<0.05.

FIG. 38. Gene transfer in pig livers treated with open UMGD using preliminary screening conditions. Resultant expression after treating using H185D, a circular planar transducer with 3 lensed cylindrical focuses at a depth of 20 mm from the face. Several US protocols were tested to determine optimal conditions to use for further testing. Each data point represents one sample of liver tissue. n=1 for all groups. Horizontal lines represent mean for each group. Error bars indicate standard error.

FIG. 39. Spatial distribution of gene transfer in pig liver treated with open UMGD. Measured luciferase expression from a representative experiment following treatment with H185D using a [200 μs, 4.6 MPa] condition. Expression is mapped onto the liver image using the underlying 1 cm grid. Cartesian coordinates for each sample were recorded during tissue sectioning prior to luciferase assay. Each point represents a core-sample of liver tissue with 1 cm² cross-sectional area (partially occupied sample squares used only the tissue falling within the square). Points are shown at the center of each sample area. Expression is listed in RLU/mg protein for each point.

FIGS. 40A-40C. Histological analysis and evaluation of serum liver transaminase enzymes (ALT and AST) in treated and control pigs show minimal tissue damage. Blood was collected from all experimental pigs 24 hours post-surgery for a complete blood count and chemistry panel. The plasma levels of enzymes, (FIG. 40A) alanine (ALT) and (FIG. 40B) aspartate-aminotransferase (AST) were examined from pigs treated with various pressure and pulse duration pairings. The treated groups analyzed were ([19 μs, 6.9 MPa]; N=8), ([200 μs, 4.6 MPa] N=5), and ([2 ms, 2.6 MPA]; n=5). All analytes were found to be within normal limits, shown as region enclosed by dotted lines, compared to sham-treated (n=2). Each circle represents one pig. Horizontal lines indicate average value within a US pairing group. Error bars indicate standard deviation. (FIG. 40C) Representative hematoxylin and eosin-stained images from treated pig livers using different US protocols are shown. (i) Untreated control and (ii) sham-treated liver was used for comparison. The liver lobe did not have direct pDNA/MB infusion or US applied to the surface. (iii & iv) Treated liver using the US protocol (19 μs, 6.9 MPa). (v & vi) Treated liver using the US protocol (200 μs, 46. MPa). (vii & viii) Treated liver using the US protocol (2 ms, 2.6 MPa). Some focal areas of hepatic injury with peri-central hemorrhage, congestion, and apoptosis. Hepatic injury and peri-central hemorrhage are indicated by black arrows. All images were obtained at 10× magnification.

FIGS. 41A-41B. Gene transfer in semi-open UMGD treated pig livers. All pigs were treated with the same concentration of pGL4 plasmid and MBs according to their weight via the segmental PV branch with simultaneous exposure of the target liver lobe to therapeutic US. For the semi-open surgeries, forceps were used to hold the folds of skin around the incision site. The US was placed on the skin, over the target liver lobe during treatment. Data is shown as representative tissue sections. (FIG. 41A) Comparison of open and semi-open surgeries to sham-treated pigs using H185D. Expression from selected US-treated sections show significant difference compared to select sham-treated sections for both surgery groups. (FIG. 41B) Comparison of resulting gene transfer using H185D to H185F for semi-open UMGD procedures. The focus of model H185F was revised to 30 mm. Absolute gene expression has been enhanced by the transducer modification. However, there is still a reduction in gene transfer efficiency when treatment occurs transcutaneously, suggesting a need for more powerful therapeutic transducers. Horizonal lines indicate average value within a group. * P<0.05, ** P<0.005.

FIGS. 42A-42C. Wave-propagation models of UMGD across different tissue layers using H185D show pressure reduction in transcutaneous application. (A) The left plot is a representation of tissue compositions in open surgery. The right plot is a representation of tissue layers present in transcutaneous UMGD. (B) The left plot is the resultant pressure field from H185D for open UMGD procedures. Target depth (portal vein) is located at ˜15 mm. The right plot is the resultant pressure field for H185D in a transcutaneous UMGD procedure. Peak US intensity does not reach the liver tissue and is instead dissipated within the muscle and fatty tissue layers. (C) Resultant pressure field simulated for H185F during transcutaneous UMGD. Though the maximal PNP magnitude achieved in the liver is comparable to the H185D case, H185F has a deeper focal depth and wider transition region, allowing it to more effectively insonify the full depth of the liver.

FIGS. 43A-43B. Pressure field plots comparing transducer H185D to H185F. (FIG. 43A) Pressure fields of H185D (left) and H185F (right) measured at 25 mm from the exit plane of the transducer. The pressure field of H185D becomes scattered when moving away from the focus (˜20 mm), whereas the focus of H185F has been shifted further (˜30 mm) to produce a more uniform pressure field at the same distance from the face of the transducer. (FIG. 43B) Pressure field plots of H185F. XY map (left) shows pressure field 30 mm from the exit plane of the transducer. YZ map (right) shows the length of the US beam where the face of the transducer is located at −25 mm on the Z axis.

FIG. 44. Prolonging pulse duration with lowered pressure procedures equivalent luciferase gene expression in semi-open UMGD treated pig livers using H185F. Data is shown as representative tissue sections. Comparison of two different US parameter pairings ([19 μs, 6.9 MPa] and [2 ms, 2.6 MPa]) to sham-treated pigs using H185F. Expression from selected US-treated sections show significant difference compared to selected sham-treated sections for both US parameter groups. There is no significant difference between the two US-treated groups. Horizontal lines indicate average value within a group. * P<0.05, ** P<0.005, N. S.=not significant.

FIG. 45: Human Factor VIII Antigen Level. Factor VIII expression persists through day 7.

FIGS. 46A-46B Evaluation of Transaminase Levels in Treated Dogs. Liver transaminase levels return rapidly to within normal range.

FIGS. 47A-47C. Imaging and treatment localization strategy. (FIG. 47A) Schematic illustration of an embodiment of the described transcutaneous UMGD in canine model. Therapeutic US (tUS) localized by guided-US and fluoroscopy is illustrated in the left lobe (FIG. 47B) and right middle lobe (FIG. 47C) of a dog.

FIGS. 48A-48C Transcutaneous UMGD of HFVIII-X10 plasmid into normal dog liver. Normal dogs (n=4) were injected with 0.67 mg/kg hFVIII-X10 plasmids mixed with 0.2 mL/kg RN18 MBs via a balloon catheter inserted into a hepatic venous branch guided by fluoroscopy. US was applied transcutaneously by scanning transducer H114 (PNP at 2.5 MPa, Pulse duration at 200 μs) on top of the dog liver lobes for 8 minutes. Two US treatments were carried out on separate liver lobes (right middle and left lobes, respectively) for each dog. (FIG. 48A) HFVIII expression over time. HFVIII expression in the normal dog plasma was analyzed by a hFVIII-specific ELISA. Each line represents an individual animal. (FIG.>48B) Representative maps of plasmid vector distribution in treated dog liver lobes. At 60 days post-surgery, tissue was sectioned and locations were recorded according to an alphanumerical grid. Samples were subsequently lysed to isolated cellular DNA, which was subsequently subjected to qPCR. Representative distribution maps of hFVIII plasmid copies in dog FLR002 liver lobes were shown (left panel, left lobe; right panel, right middle lobe). (FIG. 48C) Serum liver transaminase levels. Alanine aminotransaminase (ALT; left panel) and aspartate aminotransaminase (AST; right panel) were examined at various time points. The gray bar represents the normal ranges.

### DETAILED DESCRIPTION

Described herein are new protocols for microbubble-mediated gene and drug delivery, which include one or more percutaneous access to a target site, percutaneous delivery and capture of therapeutic compound(s) adjacent to a tissue to be treated, fluoroscopic-assisted targeting, diagnostic ultrasound-assisted targeting, microbubble insonation, and/or application of sonic waves using ultrasound transducer(s) developed herein. These protocols enable ultrasound-mediated gene and drug delivery at a scale appropriate for use in large animals, including humans.

Embodiments provided herein include a new protocol and device which enable ultrasound-mediated gene and drug delivery enhanced by destruction of microbubbles for physically targeted delivery into cells, tissues, and organs. Embodiments of the current technology overcome prior limitations to provide transcutaneous ultrasound delivery of therapeutic compounds, for instance nucleic acids in association with microbubbles, at scales appropriate for human and other large animals, without significant tissue damage. Example delivery systems are enhanced by targeted destruction of microbubbles, which increases the permeability of and thus delivery of adjacent drugs/compounds to the desired tissue(s).

Also included in various ultrasound compound delivery embodiments is a percutaneous (but only minimally invasive) surgical procedure that permits access to a target tissue site, facilitated by a combination of fluoroscopy and diagnostic US imaging. By way of example, one such percutaneous procedure is the insertion of a balloon catheter or similar device, which when inflated enables the localization of therapeutic compound(s) adjacent to the blockage so created. In some instances, the localization of the therapeutic compound(s) is determined and/or confirmed through another procedure, such as fluoroscopic or radioscopic imaging, diagnostic ultrasound, and so forth—for instance, through a detectable characteristic of the compound(s) or molecules associated therewith.

In protocols and methods provided herein, therapeutic ultrasound is used to insonate microbubbles in the presence of the therapeutic compound(s) (for instance, naked plasmid DNA (pDNA)), thereby enabling transfer into a large volume of tissue transcutaneously. By first trapping (corralling, capturing) therapeutic compound adjacent to or near (adjacent to) the tissue to be targeted by the therapeutic ultrasound—for instance, within a vein or artery that is adjacent to or within the targeted tissue or organ—the therapeutic ultrasound need only move the compound(s) from the capture region into the desired target site, for instance across the endothelial wall of the blood vessel and into the target tissue or organ.

Optionally, the placement/location of the compound(s) can be determined before the therapeutic ultrasound is performed (or concurrently therewith), for instance by detection using fluoroscopy, radiography, diagnostic ultrasound, or like methods. The specific method(s) of detection may be influenced by the type of compound(s) being used in the treatment, and compounds may be selected, modified, or mixed with detectable companion compounds in order to facilitate such detection.

Also described herein is the development of specialized ultrasound transducers which are constructed for use in transcutaneous therapeutic treatments, particularly having improved PNP output and increased treatment area. These include but are not limited to the five element, 10×80 mm (XDR106.5E) and ten element, 40×80 mm (XDR106.10E) transducers. Also provided are methods of using such transducers to target therapeutic compound delivery to tissues, cells, and organs-including such found more than a centimeter inside of an animal. In certain embodiments, the target tissue is at least 1 cm below, at least 2 cm below, at least 3 cm below, at least 4 cm below, at least 5 cm below, or more than 5 cm below the dermis of the subject. Relatively large subjects (for instance, subjects of 2 kg or more in mass) are specifically contemplated.

Lower-pressure conditions, close to the microbubble (MB) inertial cavitation threshold and focused towards further increasing gene transfer efficiency and reducing associated cell damage are developed herein. The data presented herein show that it is possible to eliminate the requirement of high PNPs by prolonging pulse durations for effective UMGD in vitro and in vivo, circumventing the peak power density limitations imposed by piezo-materials used in US transducers. Overall, these results demonstrate the advancement of UMGD technology for achieving efficient gene transfer and potential scalability to larger animal models and human application.

Significant gene transfer enhancement is described herein using targeted, ultrasound (US)-mediated gene delivery (UMGD) of non-viral vectors in large animal models via an open surgery procedure. This provides a minimally invasive treatment protocol that involves therapeutic US (tUS) across the skin for ease of clinical translation. However, gene transfer efficiency was reduced with transcutaneous UMGD due to US power attenuation across multiple tissue layers.

In addition, different US transducers and parameters were developed to overcome power loss while maintaining gene transfer efficiency. Described herein are methods that involve minimally invasive, interventional radiologic techniques combined with transcutaneous US treatment to significantly enhance gene transfer to targeted tissue (exemplified by liver lobes in live pigs). Also described are innovative US transducers which minimize power attenuation across several tissue barriers for efficient UMGD.

Though not limited to such use, methods, systems and devices described herein enable the introduction of gene modifications directly to the liver lobe(s) of large (e.g., over 2 kilogram) mammalian subjects, including humans. Such treatments are exemplified herein in the context of delivering gene therapy (such as DNA encoding a plasma Factor VIII) directly into the liver of a subject having hemophilia A, thereby treating the hemophilia.

Additional options and embodiments of the disclosure are now described in more detail.

The following sections describe information and steps to support therapeutically effective treatments involving targeted transcutaneous ultrasound compound delivery, including methods involving therapeutic compound guidance and optional capture, localization, and compound delivery/transport.

Ultrasound is recognized as acoustic energy that can be applied for imaging, for instance of structures within the body of a subject. Representative ultrasound imaging equipment is described, for instance, in Patent Publication US 2007/0255117, U.S. Pat. Nos. 6,527,718, 7,358,226, and International Patent Publication WO 2006/131840. Increasingly, ultrasound is also described as a source of external energy that can affect drug release, by altering one or more physical properties of ultrasound-sensitive carrier(s).

Ultrasound is generally applied by means of a transducer probe that sends (and receives) ultrasonic sound waves. When using ultrasound to activate drug delivery, the basic requirement is that ultrasonic waves can be transmitted into target, such as a tissue or more generally the body of a subject. Such soundwave applicators are known (see, e.g., International Patent Publication WO 2006/131840), and commercially available (see, e.g., products made by Sonic Concepts, Inc.).

Ultrasound particles are a class of particles such as microbubbles, microparticles, nanoparticles, microcapsules, and nanocapsules, having in common that they undergo a physical change upon the application of ultrasound. This change can alter characteristic(s) of the particle, including its physical state (for instance, by melting), integrity (for instance, through ultrasound-mediated destruction of microbubbles), shape/size (for instance, oscillation in size), and/or porosity (for instance, temporarily availability of the particle payload to the surrounding medium).

Particles capable of activation by ultrasound (that is, ultrasound particles) include aqueous suspensions of gaseous microbubbles. These exhibit large differences in acoustic impedance between a gas (such as air) and the surrounding aqueous medium. Such microbubbles can enhance ultrasound signals by a factor of up to a few hundreds. Detailed descriptions of the development of ultrasound contrast agents are given in the reviews by Harvey et al. (Eur. Radiol. 11:675-689, 2001) and Correas et al. (Eur. Radiol. 11:1316-1328, 2001). Ultrasound particles beneficially are small enough to be injectable intravenously and to pass through the capillaries of most tissues; thus, they are generally smaller than about 8 microns, but preferably not so small as to lose significant echogenicity. Particles of 3-4 microns are considered to be an optimal size, as they possess sufficient echogenicity but still pass through the capillaries of most tissues (Klibanov, “Ultrasound Contrast Agents: Development of the field and current status” in Topics in Current Chemistry, 222:73, Springer-Verlag Berlin, Heidelberg; 2002). In addition, size influences the optimal imaging frequency or resonance of the particle. Particles of 2 to 4 micron diameter may therefore be beneficial because their resonance lies in the medical diagnostic imaging frequency range of 1 to 10 MHz.

Microbubbles cavitate under the influence of ultrasound, as a result of which an associated or conjugated bioactive agent will be released and can thus be delivered to a target site. Microbubbles useful for drug delivery using ultrasound imaging contrast agents are described in WO 97/33474. However, many other suitable particles have been taught; see, for instance WO 2005/039750 (core-shell microparticles made by mixing a polyelectrolyte microgel and a colloid in an aqueous solution); U.S. Pat. No. 5,487,390 (gas-filled polymeric microcapsules for ultrasound imaging, formed by ionotropically gelling synthetic polyelectrolytes by contact with multivalent ions) and U.S. Pat. No. 5,562,099 (similarly constructed polymeric microcapsules filled with contrast agent); WO 89/06978 (describing ultrasonic contrast agents consisting of micro-particles containing amyloses or synthetic biodegradable polymers); EP 0441468 (ultrasound contrast agents including microparticles having a particle diameter of from 0.1 to 40 microns consisting of a biodegradable polymer obtainable from a polymerizable aldehyde and a gas and/or liquid having a boiling point of less than 60° C.); EP 0576519 (ultrasound contrast agents including gas-filled vesicles described as “microballoons” that include microbubbles of gas encapsulated by monolayers or one or more bilayers of non-proteinaceous crosslinked or polymerized amphiphilic moieties); US 2002/0150539 and US 2005/0123482 (gaseous precursor-filled liposomes suitable for use as contrast agents for ultrasonic imaging or as drug delivery agents); WO 00/72757 (surface stabilized microbubbles); WO 2007/010442 (polymeric particles, partially filled with a gas or a gas-precursor, for use in ultrasound-mediated drug delivery); US 2006/0002994 (liposomes with enhanced ultrasound responsiveness, based on the incorporation of surface active dopants containing ethylene glycol polymers or oligomers). Additional references include US 2008/0319375 (“Materials, Methods, and Systems for Cavitation-mediated Ultrasonic Drug Delivery In Vivo”); US 2008/0213355 (“Method and System for in Vivo Drug Delivery); US 2013/0261442 (“Methods and System for Ultrasound-Mediated Drug Delivery”); US 2011/0125080 (“Ultrasound Mediated Drug Delivery”).

The phrase diagnostic agent encompasses any atom, molecule, or compound that is useful in diagnosing a disease or condition. Diagnostic agents include, but are not limited to, radioisotopes, dyes, contrast agents, fluorescent compounds or molecules, and enhancing agents (such as paramagnetic ions). A non-radioactive diagnostic agent is a contrast agent suitable for magnetic resonance imaging, computed tomography, or ultrasound.

Similarly, an imaging agent refers, in the current context, to any atom, molecule or compound that is useful in detecting physical changes or that produces images of internal body tissues. In some instances, an imaging agent may also be a diagnostic agent.

The terms treatment and “to treat”, and the like, encompass therapeutic or suppressive measures for a disease or disorder leading to any clinically desirable or beneficial effect, including, but not limited to, alleviation or relief of one or more symptoms, regression, slowing or cessation of progression of the disease or disorder. Treatment can be evidenced as a decrease in the severity of a symptom, the number of symptoms, or frequency of relapse.

The terms “preventing,” “inhibiting,” “reducing” or any variation of these terms, includes any measurable decrease or complete inhibition to achieve a desired result. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99% or more, or any range derivable therein, reduction of activity or symptoms, compared to normal.

It is contemplated herein that the term subject encompasses humans as well as other animals. Thus, the term subject includes primate and non-primate, and include without limitation livestock and domestic animals, veterinary animals, and research animals.

Over the past decade, ultrasound-mediated gene delivery (UMGD) has seen advancement towards clinical translation. Ultrasound imaging can be enhanced using the echogenicity of IV-administered contrast agents, such as microbubbles. Microbubbles act as exogenous cavitation nuclei when exposed to ultrasound, meaning the MBs undergo radial oscillation and collapse. This phenomenon can be exploited to disrupt the vascular wall and cellular membranes of target cells temporarily, allowing a non-viral vector, in this case plasmid DNA, to cross and enter the cell. In this example, a luciferase-encoding plasmid DNA is used to assess the level of gene transfer, targeting the liver. The liver is targeted because it is a primary site of Factor VIII production, the blood clotting factor deficient or lacking in Hemophilia A patients. Thus, UMGD as developed here can be used to deliver Factor VIII-encoding pDNA to treat hemophilia A patients. However, it is also contemplated for use with delivery of other compounds or compositions (generally agents, including therapeutic agents), for the treatment of additional conditions and diseases, and in various modified methods and systems.

An ultrasound dosage form is an ultrasound particle that includes a therapeutic agent. The therapeutic agent can be associated with and/or bound to the ultrasound particles, for instance covalently (by chemical interaction) or by physical interaction (such as adsorption). In some embodiments, the therapeutic agent is incorporated into a cavity of the ultrasound particle.

Generally, a therapeutic agent is an atom, molecule, or compound that is useful in preventing and/or treating a disease or condition. In the current context, the term specifically encompasses any bioactive agent that is useful to be administered using ultrasound. This includes agents that treat a disease or disorder (treatment agents), as well as agents that prevent the occurrence, or worsening, of a disease or disorder (prophylactic agents). The term also includes genetic material, including DNA (such as plasmid DNA) and RNA (such as siRNA and in vitro transcribed mRNA).

Accordingly, compounds envisaged for use as bioactive agents in the context of the present disclosure include any compound with one or more therapeutic or prophylactic effects. Such compounds include those which affect or participate in tissue growth, cell growth, cell differentiation; which are able to invoke a biological action such as an immune response; as well as compounds that can play any other role in at least one biological process. A non-limiting list of examples includes antimicrobial agents (including antibacterial, antiviral agents and anti-fungal agents), anti-viral agents, anti-tumor agents, thrombin inhibitors, anti-thrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, anti-mitotics, microtubule inhibitors, anti-secretory agents, actin inhibitors, remodeling inhibitors, anti-metabolites, anti-proliferatives (including anti-angiogenesis agents), anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, ACE inhibitors, free radical scavengers, chelators, antioxidants, anti-polymerases, and photodynamic therapy agents.

Any composition formulation disclosed herein can advantageously include any other pharmaceutically acceptable carriers which include those that do not produce significantly adverse, allergic, or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol, or mannitol.

Exemplary stabilizers include organic sugars, polyhydric sugar alcohols, polyethylene glycol; sulfur-containing reducing agents, amino acids, low molecular weight polypeptides, proteins, immunoglobulins, hydrophilic polymers, or polysaccharides.

Combinations of active components and/or device components can be provided as kits. Kits can include containers including one or more or more ultrasound particles as described herein, optionally along with one or more targeting, therapeutic, or diagnostic agents. For instance, some kits will include at least ultrasound-sensitive microbubble preparation, along with an amount of at least one nucleic acid or other therapeutic compound, formulated to be administered to a subject. Optionally, kits will include a device useful for percutaneous localization of the compound(s) to be delivered via transcutaneous ultrasound. Such device may for instance be a balloon catheter or component thereof. Any active component in a kit may be provided in premeasured dosages, though this is not required; and it is anticipated that certain kits will include more than one dose.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding preparation of polynucleotides (PN) or nanoparticles (NP), for administration; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as syringes, ampules, tubing, facemask, an injection cap, sponges, sterile adhesive strips, Chloraprep, gloves, and the like. Variations in contents of any of the kits described herein can be made. The instructions of the kit will direct use of the active ingredients to effectuate a new clinical use described herein.

Unless otherwise indicated, the practice of the present disclosure can employ conventional techniques of immunology, molecular biology, microbiology, cell biology and recombinant DNA. These methods are described in the following publications. See, e.g., Sambrook et al., Molecular Cloning: A Laboratory Manual, 2nd Edition (1989); Ausubel et al., eds., Current Protocols in Molecular Biology (1987); the series Methods in Enzymology (Academic Press, Inc.); M. MacPherson, et al., PCR: A Practical Approach, IRL Press at Oxford University Press (1991); MacPherson et al., eds. PCR 2: Practical Approach (1995); Harlow and Lane, eds. Antibodies, A Laboratory Manual (1988); and Freshney, ed. Animal Cell Culture (1987).

### Exemplary Embodiments

A first embodiment provides transcutaneous, ultrasound-mediated delivery methods for administering a therapeutic compound to a target tissue in a subject, the method involving positioning a balloon catheter (or other positionable occluding device) in a blood vessel of the subject such that the balloon (or blockage) is adjacent to the target tissue; inflating the balloon catheter to occlude outflow from a region adjacent to the target tissue; administering the therapeutic compound to the vessel of the subject such that it is substantially retained adjacent to the target tissue by the balloon catheter; determining the location of the therapeutic compound and/or a detectable adjunct compound (such as an ultrasound contrast agent, radioisotope, or the like) administered therewith using at least one of diagnostic ultrasound, radiography, or fluorography; administering therapeutic ultrasound energy (sonication) transcutaneously, such that the energy mediates delivery of the therapeutic compound across the vessel wall and into the adjacent target tissue. Optionally, such methods further involve administering to the subject a composition comprising a coagulation factor to the subject before the therapeutic ultrasound energy (sonication) is administered to the subject.

In examples of the transcutaneous, ultrasound-mediated delivery methods, the therapeutic compound and/or the adjunct compound comprises microbubbles (MBs).

In examples of the transcutaneous, ultrasound-mediated delivery methods, the therapeutic compound comprises a nucleic acid molecule capable of expression in at least one cell type in the target tissue. By way of example, the nucleic acid molecule can include one or more naked plasmid DNA encoding at least one peptide, protein, or functional RNA molecule.

Optionally, in any of the provided methods the transcutaneous therapeutic sonication is performed using parameter pairings between the 1 and 3 J energy curves. The therapeutic sonication beneficially is performed using parameter pairings that can generate effective energy for efficient MB cavitation and gene transfer, as described.

In examples of the transcutaneous, ultrasound-mediated delivery methods, the transcutaneous therapeutic sonication is performed using an ultrasound transducer selected from model H114, XDR106-5E, and XDR106-10E.

The transcutaneous therapeutic sonication in example methods is performed with a frequency of about 0.5-3 MHz. It may be performed for a period of between about 10 seconds and about 15 minutes in any of the provided methods.

The methods provided herein are of particular use for delivery of therapeutic compound(s) to tissue within a relatively large subject (for instance, a subject of 2 kilograms or more). Another way to look at this is that the described methods provide for delivery to target tissue that is at least 1 cm below, at least 2 cm below, at least 3 cm below, at least 4 cm below, at least 5 cm below, or more than 5 cm below the dermis of the subject.

It is particularly expected that the provided methods will be used to deliver compounds (such as expressible nucleic acids) to liver tissue. Specific exemplars are described herein.

In addition, the targeted tissue in some embodiments is a tumor tissue. By way of non-limiting example, the tumor tissue can be brain tumor tissue, ovarian tumor tissue, breast tumor tissue, liver tumor tissue, kidney tumor tissue, head tumor tissue, neck tumor tissue, colon tumor tissue, or a combination thereof.

Yet another embodiment provides transcutaneous ultrasound-mediated drug delivery systems for use with any of the herein-provided subcutaneous delivery methods, the system including at least an ultrasound apparatus, a therapeutic compound capable of being administered to the target tissue, and a balloon catheter (or other temporary occluding device). In examples of this system, the ultrasound apparatus includes a function generator for generating the sonication; an amplifier connected with the function generator to amplify the sonication; a power meter connected with the amplifier; and a transducer connected between the power meter and a removable surface for transferring the sonication to therapeutic compound adjacent to the target tissue, wherein the transducer is selected from model H114, XDR106-5E, and XDR106-10E. Optionally, the provided systems may further include an ultrasound contrast agent administered to the subject before or in conjunction with administration of the therapeutic compound, wherein the ultrasound contrast agent includes microbubbles.

It is also contemplated that provided systems may further include a diagnostic ultrasound device, radiography device, or fluorography device for determining the location of the therapeutic compound and/or a detectable adjunct compound administered therewith.

In examples of any of the provided systems, the targeted tissue may be liver tissue.

In additional examples of any of the provided systems, the targeted tissue is a tumor tissue. By way of non-limiting example, the tumor tissue is a brain tumor tissue, ovarian tumor tissue, breast tumor tissue, liver tumor tissue, kidney tumor tissue, head tumor tissue, neck tumor tissue, colon tumor tissue, or a combination thereof.

Also provided are methods for transcutaneous ultrasound treatment to tissue internal to a subject, such methods including: obtaining percutaneous access to a target site within the subject, percutaneous delivery and capture of administered therapeutic compound(s) adjacent to a tissue to be treated at the target site, fluoroscopic-assisted and/or diagnostic ultrasound-assisted targeting of the therapeutic compound(s), and microbubble insonation at the target site using transcutaneously ultrasound, thereby releasing the therapeutic compound(s) into tissue at the target site. Optionally, in such methods the transcutaneous ultrasound insonation is applied using a model H114, XDR106-5E, or XDR106-10 ultrasound transducer.

Specific methods described herein employ a therapeutic US (tUS) device that provides the following combination of features: small form factor, ergonomic to subject, high effective treatment volume, good penetration, operation at 1 MHZ, and high peak pressures with low duty cycle. Specifically, such a tUS is used to insonicate microbubbles transcutaneously, for instance where such microbubbles have been localized in the subject and that localization confirmed using one more of diagnostic ultrasound, fluorography, or radiography.

### Example 1. Prolonging Pulse Duration in Ultrasound-Mediated Gene Delivery Lowers the Acoustic Pressure Threshold for Efficient Gene Transfer to Cells and Small Animals

This Example describes development of clinically applicable procedures involving transcutaneous ultrasound-mediated gene deliver (UMGD). At least some of the material described in this example was published in Tran et al., J Controlled Release 279:345-354, 2018. Introduction. Non-viral gene therapy confers appreciable benefits over viral methods including lower risk of immunopathogenicity, greater flexibility in vector construction, and better spatial and temporal control. Delivery of plasmid DNA (pDNA) is particularly attractive as manipulation of the host genome can be avoided and the vector can more easily be engineered for episomal persistence and long-term promoter activation. Ultrasound (US)-mediated gene delivery (UMGD) has long been recognized as a potential method to perform minimally invasive, non-viral gene transfer of pDNA. Effective UMGD requires the presence of microbubbles (MBs), which has been demonstrated to significantly enhance gene transfer efficiency, resulting in increased transgene expression. Under appropriate acoustic pressures and applied frequencies, spontaneous formation of gas cavities, termed cavitation, may occur. MBs serve as cavitation nuclei and can oscillate radially and collapse when exposed to a driving pressure field. Although the precise mechanism is not yet known, MB cavitation and/or destruction during therapeutic sonication is shown to facilitate transient pore formation along the cell membrane (De Cock et al., J Control Release 197:20-28, 2015; Hallow et al., Ultrasound Med Biol. 32:1111-1122, 2006). Acoustic cavitation of MBs may also increase permeability of endogenous barriers such as the cell membrane or vessel wall to allow normally impermeable materials (e.g., drugs or macromolecules) to cross via diffusion.

Other non-viral gene therapies developed include systemic exposure to lipid nanoparticles carrying genetic material or direct injection of gene vectors to tissue-specific sites (e.g., intraparenchymal or intramuscular). However, use of lipid or polymer encased pDNA may be hindered by difficulty in packaging, expelling genetic load, and avoiding cytoplasmic degradation. In addition, direct injection to tissue-specific sites faces the challenge of traversing the plasma membrane of target cells. Alternatively, systemic administration of genetic vectors is also challenged by the multiple barriers hindering entry of pDNA into cells. The vectors must first cross the endothelium, the basement membrane, and smooth muscle layer before overcoming the outer cell membrane of the target cells and finally the nuclear membrane for efficient gene expression. UMGD may potentially overcome some or all of these barriers to significantly improve gene transfer efficiency targeting specific tissue/cells. Incorporation of UMGD methods with non-viral gene therapy is motivated by the aim of treating genetic diseases, such as hemophilia, that is safe with comparable efficiency to viral gene transfer methods.

The liver is an ideal target for gene therapy in hemophilia A patients, as it is a predominant site of factor VIII production, and where deficiency of the protein is responsible for the hemophiliac phenotype. It has been shown previously that UMGD can significantly enhance gene transfer in livers of both small and larger animal models. In those mouse models, MBs and pDNA encoding a luciferase reporter gene (pGL40 were co-administered by injection into the portal vein (PV) while US was simultaneously applied to the liver lobes. However, it was necessary to modify the treatment protocol in rats to accommodate the larger liver lobes; this modification was accomplished by injecting the pGL4/MBs into individual liver lobes through a PV branch. Similar to the rat studies, a nearly 100-fold increase in average luciferase expression relative to sham was achieved when the single lobe injection strategy was translated to dog models. In agreement with those small animal studies, from an in vivo dog model it appeared that a peak negative pressure (PNP) of about 2.7 MPa was required for effective gene transfection with minimal liver tissue damage. However, the surgical procedure required opening the cavity of the animal model to treat the surface of the liver.

A clinically applicable procedure would involve transcutaneous UMGD. Given the 2.7 MPa threshold necessary for effective gene transfer into the liver cells however, acoustic pressures beyond the capabilities of the standard piezo-material would be required due to the loss of acoustic energy passing through several tissue layers. While UMGD has historically been achieved through high PNPs of 2 MPa or greater, emerging evidence suggests it may not be a requirement for MB cavitation (Lin et al., Ultrasonics Sonochemistry 35:176-184, 2017; Chen et al., Ultrasound Med Biol. 42:528-538, 2016; Apfel & Holland, Ultras Med Biol. 17:179-185, 1991).

In this example, lower-pressure conditions were investigated for UMGD, close to the MB inertial cavitation threshold. A matrix of pulse durations and acoustic pressures was examined in HEK293T cells and mice. These conditions were focused towards achieving comparable, and possibly further increasing, gene transfer efficiency while reducing associated cell damage. Lower pressure, longer pulse duration conditions within a range of acoustic energies have been identified that produced gene expression levels similar to or better than previously reported results, without significantly increasing tissue damage. Thus, these prolonged pulse duration US protocols have potential for improving US transducer design to readily achieve US intensity threshold with increased treatment volumes that could lead to effective gene transfer transcutaneously. These results overcome current challenges in broad applications of UMGD and transducer design to facilitate clinical translation.

### Materials and Methods.

Plasmid Preparation: The luciferase reporter plasmid pGL4.13 [luc2/SV40] (Promega, Madison, WI) was produced by GenScript Inc. (Piscataway, NJ) according to standard techniques. The green fluorescent protein (GFP) reporter plasmid p2X-GFP (pGFP) was prepared as previously described (Miao et al., Hum Gene Ther. 16:893-905, 2005; incorporated herein by reference) using an Endo-Free Maxi-Prep kit (Qiagen Inc., Valencia, CA), according to the manufacturer's manual.

Microbubble Preparation: RN18 microbubbles were prepared according to the previously described protocol (Sun et al., J Control Release, 182:1111-120, 2014; incorporated herein by reference). The MB shells were composed of lipids at a 82:10:8 molar ratio of 1,2-distearoyl-sn-glycero-3-phosphocholine (DSPC), 1,2-distearoyl-sn-glycero-3-phosphate (DSPA), and N-(Carbonylmethoxypolyethyleneglycol 5000)-1,2-distearoyl-sn-glycero-e-phospho-ethanolamine (MPEG-5000-DSPE) purchased from Avanti® Polar Lipids, Inc. (Alabaster, AL). Before use, MBs were produced by vigorous agitation of the lipid emulsion for 45 seconds using a Vialmix™ (Lantheus Medical Imaging, N. Billerica, MA), yielding an average concentration of 2-5×10⁹ MBs/mL.

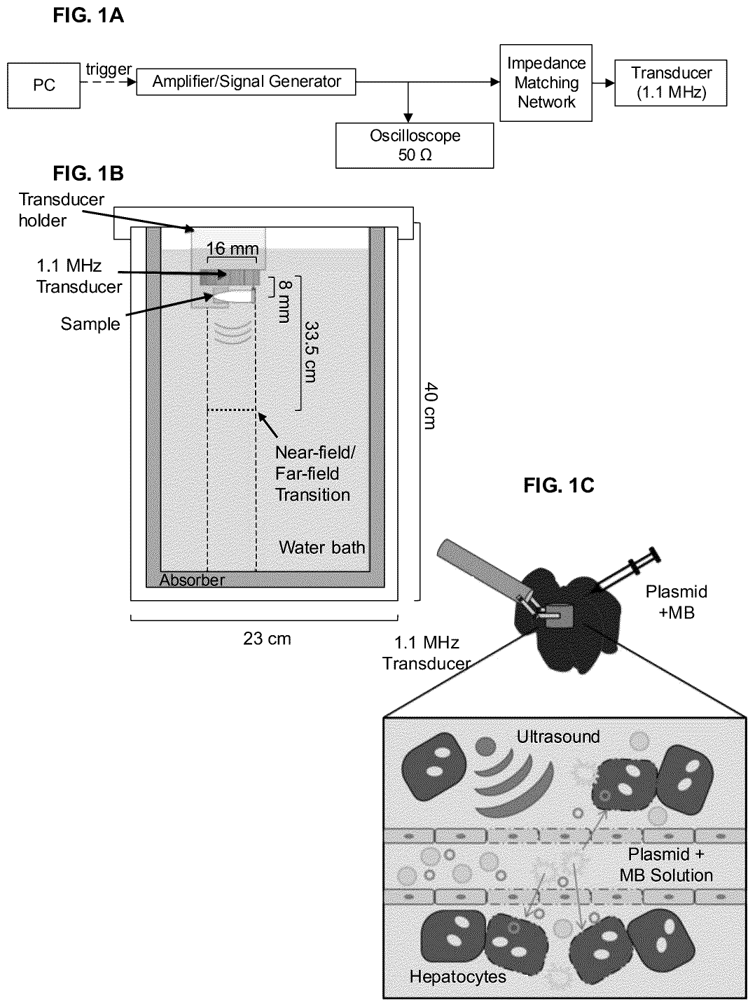

Ultrasound Conditions: The experimental ultrasound setup for both cell culture and murine experiments is summarized in FIG. 1A. A laptop was used to control a signal-generating amplifier via serial interface (Models: RFG-1000 and RFG-1500BB, JJ&A Instruments, Duvall, WA) that was connected to an impedance matching network and subsequently to a single-element, 16 mm diameter, unfocused transducer (Model H158, Sonic Concepts, Bothell, WA). A broad range of ultrasound conditions was tested in both HEK293T cells (Table 1) and mice (Table 2).

TABLE 1
Summary of HEK293T experimental conditions

| Pulse | | PNP | Power | |
|---|---|---|---|---|
| Duration | | (MPa) | (W) | n |
| 18 | μs | 2.5 | 280 | 8 |
| 50 | μs | 0.5 | 10 | 3 |
| | | 1.1 | 50 | 3 |
| | | 1.5 | 100 | 4 |
| | | 2.1 | 200 | 4 |
| 150 | μs | 0.7 | 20 | 5 |
| | | 1.1 | 50 | 5 |
| | | 1.5 | 100 | 7 |
| | | 2.1 | 200 | 7 |
| 400 | μs | 0.5 | 10 | 5 |
| | | 0.7 | 20 | 5 |
| | | 1.1 | 50 | 5 |
| | | 1.5 | 100 | 6 |
| | | 2.1 | 200 | 7 |
| 1 | ms | 1.3 | 71 | 5 |
| | | 2.2 | 210 | 5 |
| 2 | ms | 0.5 | 10 | 3 |
| | | 0.7 | 20 | 2 |
| | | 0.8 | 30 | 2 |
| | | 1.1 | 50 | 5 |
| | | 1.6 | 110 | 5 |
| | | 2.0 | 180 | 5 |
| 4 | ms | 0.5 | 10 | 2 |
| | | 0.7 | 20 | 7 |
| | | 0.8 | 30 | 2 |
| | | 1.1 | 50 | 6 |
| | | 1.4 | 89 | 5 |
| 5 | ms | 0.6 | 14 | 5 |
| 7 | ms | 0.5 | 10 | 5 |
| | | 1.1 | 51 | 5 |
| 11 | ms | 0.7 | 19 | 6 |
| 14 | ms | 0.6 | 15 | 5 |
| 18 | ms | 0.7 | 20 | 5 |
| 22 | ms | 0.5 | 10 | 5 |
| 24 | ms | 0.6 | 15 | 5 |
| 36 | ms | 0.5 | 10 | 5 |

TABLE 2
Summary of murine experimental conditions

| Pulse | | PNP | Power | |
|---|---|---|---|---|
| Duration | | (MPa) | (W) | n |
| 18 | μs | 0.7 | 20 | 5 |
| | | 1.1 | 50 | 5 |
| | | 1.5 | 100 | 5 |
| | | 2.1 | 200 | 5 |
| | | 2.5 | 280 | 41 |
| 50 | μs | 0.5 | 10 | 5 |
| | | 0.7 | 20 | 5 |
| | | 1.1 | 50 | 5 |
| | | 1.5 | 100 | 5 |
| | | 2.1 | 200 | 5 |
| 150 | μs | 0.7 | 20 | 5 |
| | | 1.1 | 50 | 5 |
| | | 1.5 | 100 | 5 |
| | | 2.1 | 200 | 5 |
| 400 | μs | 0.5 | 10 | 5 |
| | | 0.7 | 20 | 5 |
| | | 1.1 | 50 | 10 |
| | | 1.5 | 100 | 5 |
| 1 | ms | 0.5 | 10 | 5 |
| | | 0.7 | 20 | 10 |
| | | 0.8 | 30 | 10 |
| | | 1.1 | 50 | 5 |
| | | 1.5 | 100 | 4 |
| 2 | ms | 0.5 | 10 | 5 |
| | | 0.7 | 20 | 4 |
| | | 0.8 | 30 | 10 |
| | | 1.6 | 108 | 6 |
| 4 | ms | 0.5 | 10 | 4 |
| | | 0.7 | 20 | 9 |
| | | 1.1 | 54 | 5 |
| | | 1.4 | 94 | 5 |
| 7 | ms | 0.5 | 10 | 4 |
| 22 | ms | 0.5 | 10 | 10 |

All experiments used a center frequency of 1.1 MHz, pulse repetition frequency (PRF) of 14 Hz, and 20 cycles of a 1s ON, 2s OFF pulse train to yield 60s total treatment time. US treatment monitoring was performed by capturing current, voltage, and calculated power from a gated, triggered waveform using a high sample-rate oscilloscope (44MXs-B, Teledyne Lecroy, Chestnut Ridge, NY).

The acoustic energy (E) is defined here as the product of spatial average intensity (I_{avg}) and exposure time (T):

$${I}_{avg} = \frac{{P}_{avg}}{A} \qquad ( 1 )$$

$$⊤ = P D \cdot P R F \cdot T \qquad ( 2 )$$

$$E = {I}_{avg} \cdot τ \qquad ( 3 )$$

where P_{avg} is the average power output, A is the active cross-sectional area of the beam, PD is the pulse duration, PRF is the pulse repetition frequency and T is the insonation time. The exposure time (T) is defined here as the time length in which the ultrasound is actively on, whereas the insonation time (T) is defined as the full time duration of pulsed ultrasound treatment from start to finish (Ultrasound in Med & Biol. 35:847-860, 2009).

In vitro UMGD: Human embryonic kidney (HEK) 293T cells (ATCC, Manassas, VA) were cultured in Dulbecco's modified Eagles medium (DMEM) (Mediatech, Inc, Manassas, VA) with 10% fetal bovine serum (FBS) (Atlanta Biologicals Inc, Lawrenceville, GA), 1% HEPES buffer (Mediatech, Inc), 1% Penicillin/Streptomycin (Mediatech, Inc), and 1% L-Glutamine (Mediatech, Inc). For transfection experiments, the cells were suspended in 1.7 mL complete media and 3.2 μg of pGFP plasmid were added and then transferred into TPX microcentrifuge tubes (Diagenode Inc., Denville, NJ) leaving a small headspace of gas necessary to avoid MB destruction when capping. Subsequent steps were performed serially by each tube to prevent passive decay of diluted MBs. 0.1% (v/v) (17 μL) of activated RN18 were added into each tube, capped immediately, and mounted in the acoustic water bath.

The cell/pDNA/MB suspension was then exposed to US in the nearfield of the H158 transducer for 60s, varying the US signals used as detailed in section 2.3. The experimental set-up can be visualized in FIG. 1B. After completion, cells were gently mixed by pipetting then plated in single-cell suspension into 6-well tissue culture plates. For each experiment, pDNA transfection controls were also generated to validate gene expression by directly transferring 1.7 mL of the cell and plasmid solution to an empty well of a 6-well plate and adding 2 μL of Lipofectamine 2000 (Thermo Fisher Scientific, Waltham, MA). An additional 1.7 mL of complete media was added to each well for a total volume of 3.4 mL. Cells were harvested at 48 hours post-transfection for expression analysis via flow cytometry.

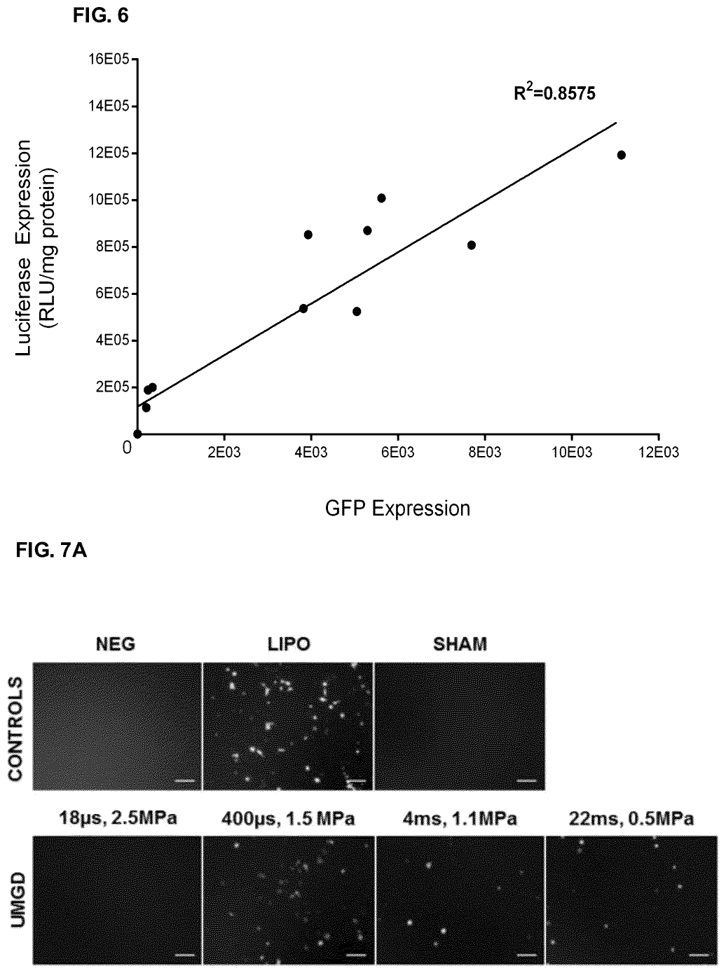

Flow Cytometry Analysis: A more robust method of assessing both viability and gene transfer efficiency was required. Flow cytometry allows the opportunity to simultaneously observe both experimental dimensions within a single experiment. Fluorescence is not a frequently reported output measure for gene transfer. Therefore, a correlation between GFP+expression and luciferase activity was determined (FIG. 6). A correlation of 0.857 was derived between GFP+expression and luciferase activity, deeming satisfactory for comparative analysis.

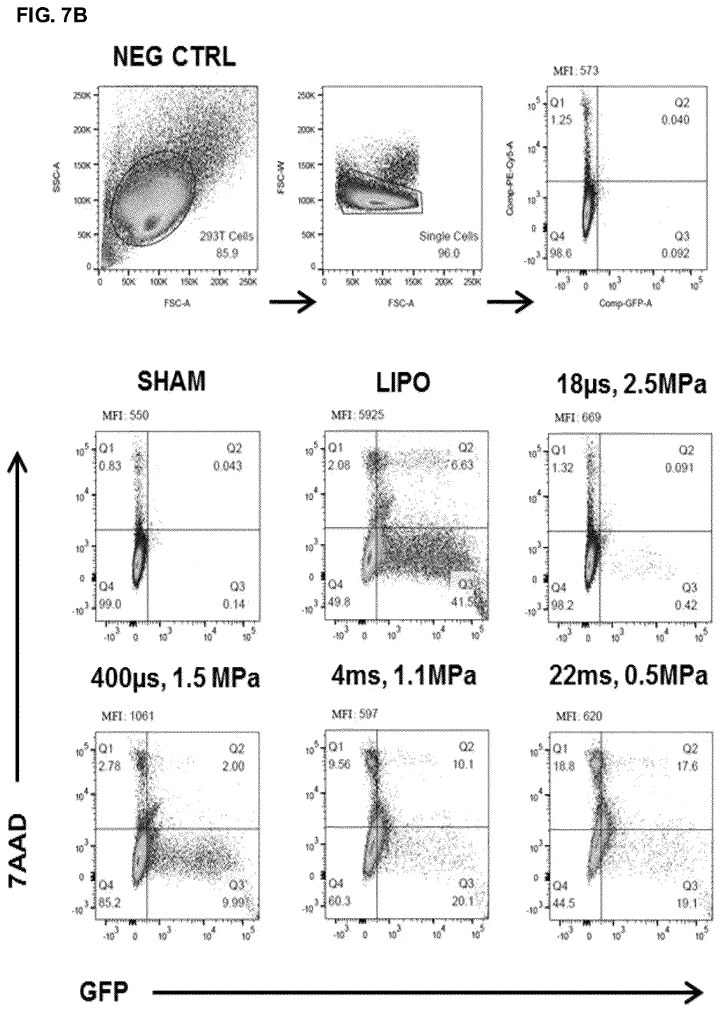

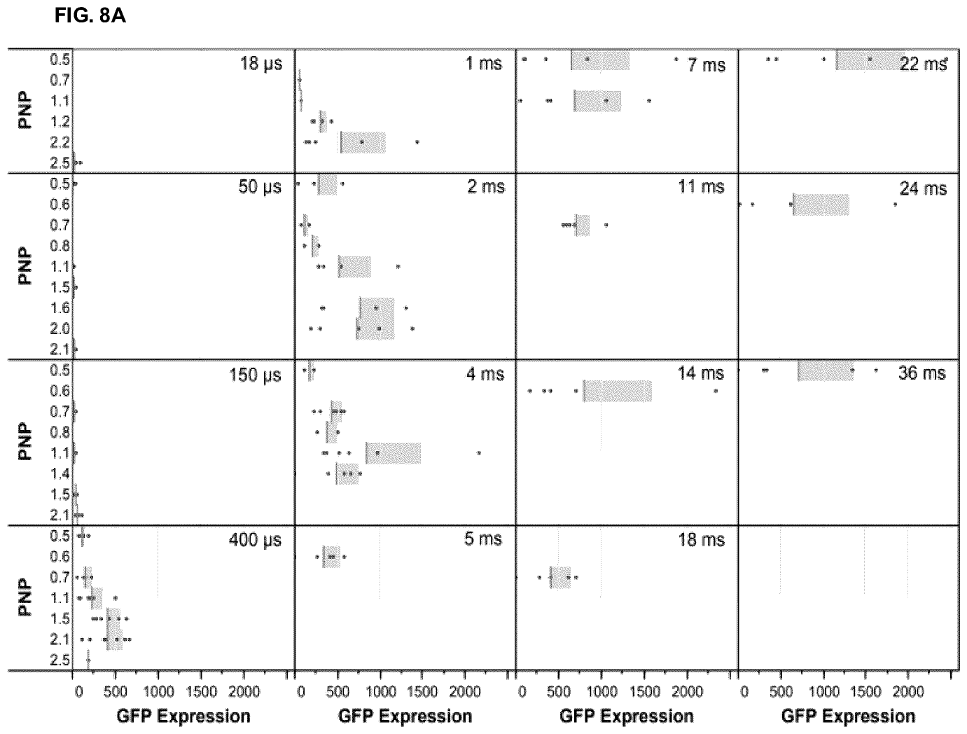

HEK293T cells were stained with a viability dye, 7AAD (Biolegend®, San Diego, CA), and analyzed via flow cytometry for PE fluorescence. GFP expression was analyzed using the FITC channel. Viability was calculated as the percentage of all events that are GFP+/−and 7AAD-events within a parent gate of singlet HEK293T cells. GFP expression was calculated as the percentage of GFP+/7AAD-events within a parent gate of singlet HEK293T cells, multiplied by the GFP MFI of the same population. Representative flow analysis and microscopy images are shown (FIG. 7A-7B). For comparison across experiments performed on different dates, GFP+expressions were normalized to a positive US control protocol (2 ms, 1.1 MPa).

In Vivo UMGD: Eight-week-old C57/BL6 male mice were purchased from the Jackson Lab (Bar Harbor, ME) and maintained at a specific pathogen-free vivarium. All animal experiments were performed in accordance with IACUC-approved protocol and guidelines for animal care of both National Institutes of Health and Seattle Children's Research Institute. Prior to surgery, mice were anesthetized by continuous inhalation of isoflurane. A midline incision was made to expose the liver and portal vein in mice. Mouse livers were transduced, varying the US signals used as detailed in section 2.3. Murine UMGD was performed as previously described (Song et al., Gene Ther., 18:1006-1014, 2011; incorporated herein by reference). Briefly, a 400 μL mixture containing 50 μg of pGL4.13, RN18 MBs, and 5% glucose in PBS was injected into the liver via the portal vein. US treatment was performed simultaneously to injection, and continued for 30s after completion of injection for a total treatment time of 60s. Hemostasis was applied and the incision site closed, and the mice were allowed to recover from anesthesia.

Evaluation of Luciferase Gene Expression: All liver lobes were harvested from the animal 24 hrs after gene delivery to analyze luciferase gene expression. The liver was subsequently processed and assayed as previously described (Noble et al., Mol Ther. 21:1687-1694, 2013; incorporated herein by reference). Briefly, supernatant was collected from homogenized liver that underwent three freeze-thaw cycles. Luciferase activity in the lysate was measured by using a commercial kit (Luciferase Assay System, Promega) and luminometer (Victor 3; Perkin-Elmer, Wellesley, MA), and then normalized to protein levels measured using a BCA assay kit (Bio-Rad, Hercules, CA). Luciferase expression data are expressed as relative light units per mg protein (RLU/mg protein).

Transaminase Assay: Blood samples were collected by retro-orbital bleeding prior to euthanizing the animal and used to assess liver damage by measuring alanine aminotransferase (ALT or SGPT) and aspartate aminotransferase (AST or SGOT) levels using commercial assay kits (Teco Diagnostics, Anaheim, CA). Normal, untreated mice were used as controls.

2.9 pDNA Stability Assessment: The stability of the pGL4.13 during US and in the presence of MBs was assessed using the same experimental set-up as the in vitro UMGD experiments. A 1.7 mL solution mixture containing 5% glucose, 50 μg of pDNA, and 5% (v/v) RN18 MBs in 1×PBS was prepared in a 1.7 mL TPX microcentrifuge tube. Using transducer H158, the mixture was exposed to US using a pulse duration of 18 μs at 2.5 MPa for 60s. The condition was repeated without MBs, and again without US+MBs. The pDNA for each condition was then linearized with Bgl II and analyzed alongside the undigested form on a 1% (v/v) agarose gel.

Cavitation Experiments: 17 μL of activated RN18 MBs was added to 1.7 mL of PBS in TPX microcentrifuge tubes. These tubes were then exposed to ultrasound identically to the in vitro protocol. The MBs used for each experimental tube were drawn from the same pool of activated RN18 MBs. After completion of ultrasound exposure, MBs were analyzed using flow cytometry and their sizes and concentrations were calibrated using known dilutions of polybeads (Polysciences Inc., Warrington, PA) on the same day. Run times were fixed and the number of events gathered was allowed to vary. Total events of US exposed MBs were compared to a control tube without US exposure. Consistent cytometer flow rate was verified by comparing dilutions of polybeads and untreated RN18 MBs from the start and end of each experiment.

Statistical Analysis: All statistical analyses were performed using a two-tailed Student's t-test with unequal variance.

### Results.

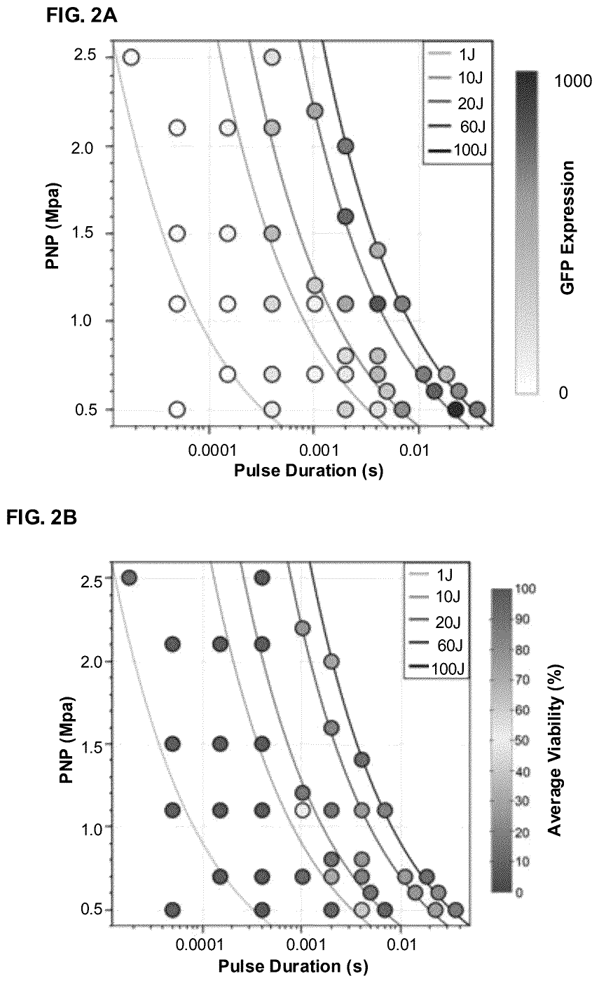

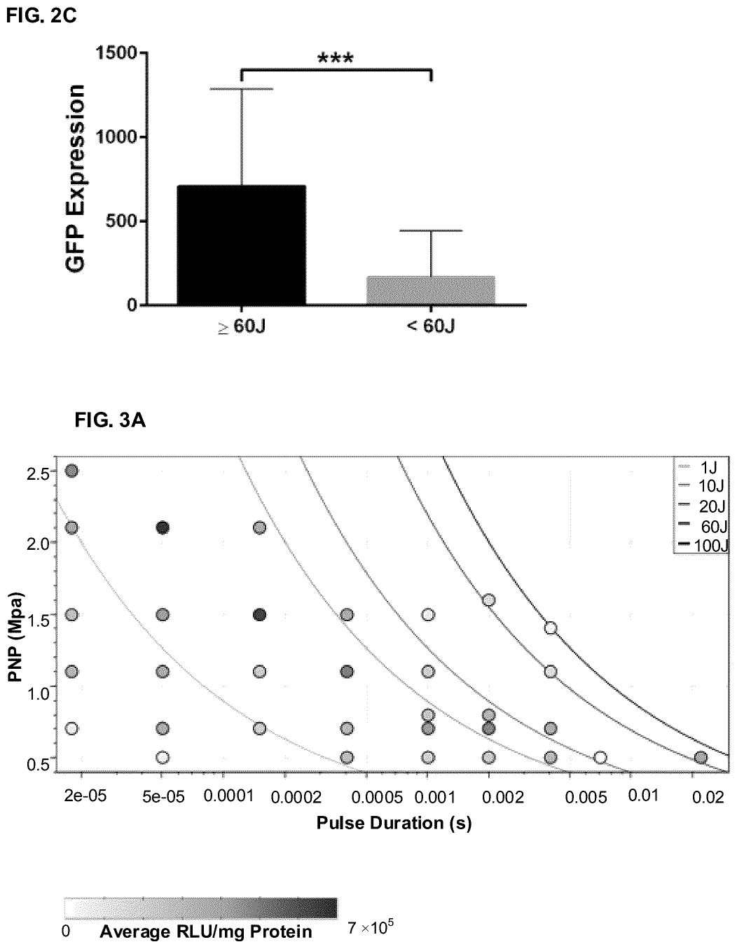

HEK293T UMGD: In order to establish a reference for ultrasound parameters in in vitro experiments, a matrix of 36 conditions was tested in HEK293T cells. These conditions spanned pulse durations of 18 μs-36 ms and PNPs of 0.5-2.5 MPa (intensities of 5-140 W/cm²). Group sizes were n=2-8 samples per condition, with a mode size of n=5 samples. GFP expression was quantified as the population of live, expressing cells and adjusted to incorporate the MFI of the population. To assess the effectiveness of varying US parameter combinations, the resulting GFP expression was plotted against pulse duration and PNP as shown in FIG. 2A. It was observed that conditions with pulse durations 150 μs and below displayed minimal increase in expression compared to sham-treated controls (no significant difference, P-value≥0.05) at all tested powers. Across most other tested pulse durations, one or more corresponding PNPs did not produce significantly different expression levels from the global expression maximum (located at 22 ms, 0.5 MPa).

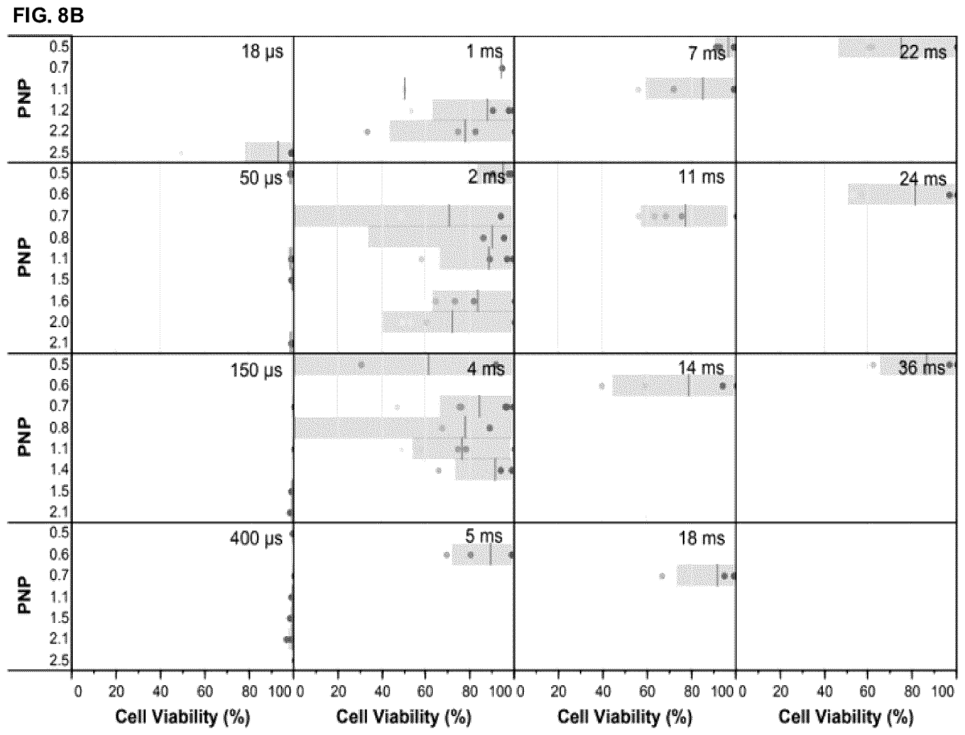

With increasing pulse duration, maximum expression was found to occur at progressively lower PNPs. Increasing PNP beyond the local expression maximum for pulse durations on the millisecond scale does not enhance expression any further; rather, expression appears to decrease. Additionally, increasing pulse duration resulted in a narrower range of optimal pressures. The majority of high-expressing conditions delivered total treatment energies greater than or equal to 60J. Expression from various US protocols appears to be separated by two treatment energy ranges. Results from US transfected HEK 293T cells were pooled into two separate groups: cells treated with acoustic energies of greater than or equal to 60J, or cells treated with acoustic energies below 60J. The pooled data from each group was averaged and compared. Analysis revealed that points of expression equal to or above the 60J treatment energy curve were significantly greater than points that lie below the 60J energy curve (P<0.0005, FIG. 2C). Mean values and standard deviation evaluations for GFP expression may be found in FIG. 8A.

HEK 293T Viability: Cell viability was quantified using flow cytometry populations selected via a 7-AAD stain. FIG. 2B shows the resulting empirical distribution of cell viability over the chosen pulse duration-PNP parameter space. The control US protocol (2 ms, 1.1 MPa) resulted in 88% cell viability. Shorter pulse durations within the microsecond scale did not affect cell viability even when increasing PNP. Cell viability begins to decrease gradually when increasing pulse duration beyond the microsecond scale. The global viability minimum occurs at a condition of 1 ms, 1.1 MPa with 50% of viable cells remaining. When plotted against total treatment energy across the parameter space, cell viability appears to decrease with increasing treatment energy. Mean values and confidence interval evaluations are shown in FIG. 8B.

Murine UMGD: The effects of 32 unique ultrasound conditions were examined in C57/BL6 mice using the existing UMGD protocol. The conditions were selectively narrowed from the HEK 293T transfection experiments and upper limits of 22 ms for pulse duration and 2.5 MPa for PNP were determined. Group sizes ranged from n=2-14 samples per condition, with a mode size of n=5 samples. The reference condition comprised of n=37 mice (18 μs, 2.5 MPa), and was used to verify stationarity across experimental days.

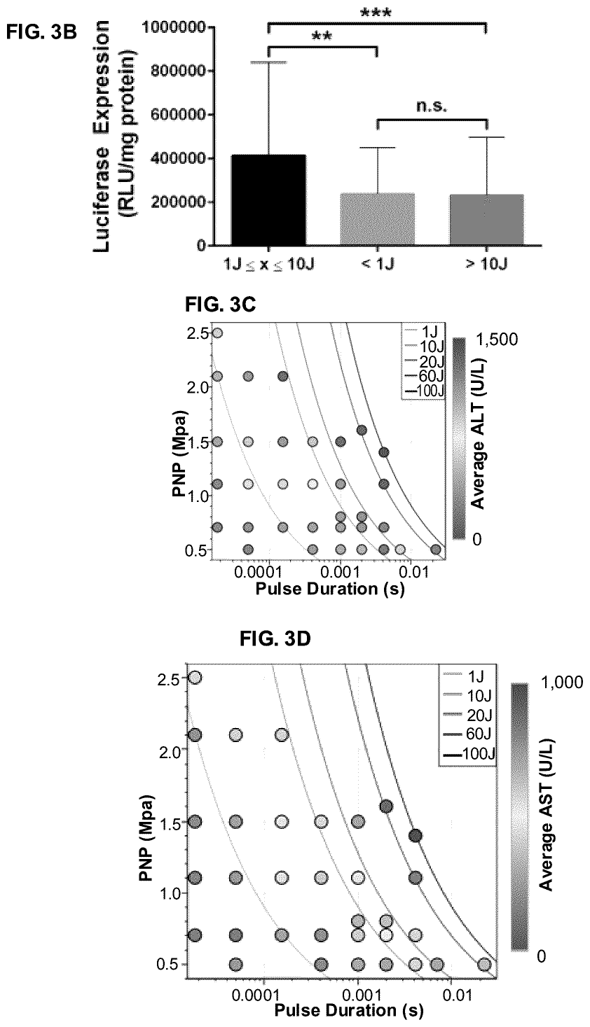

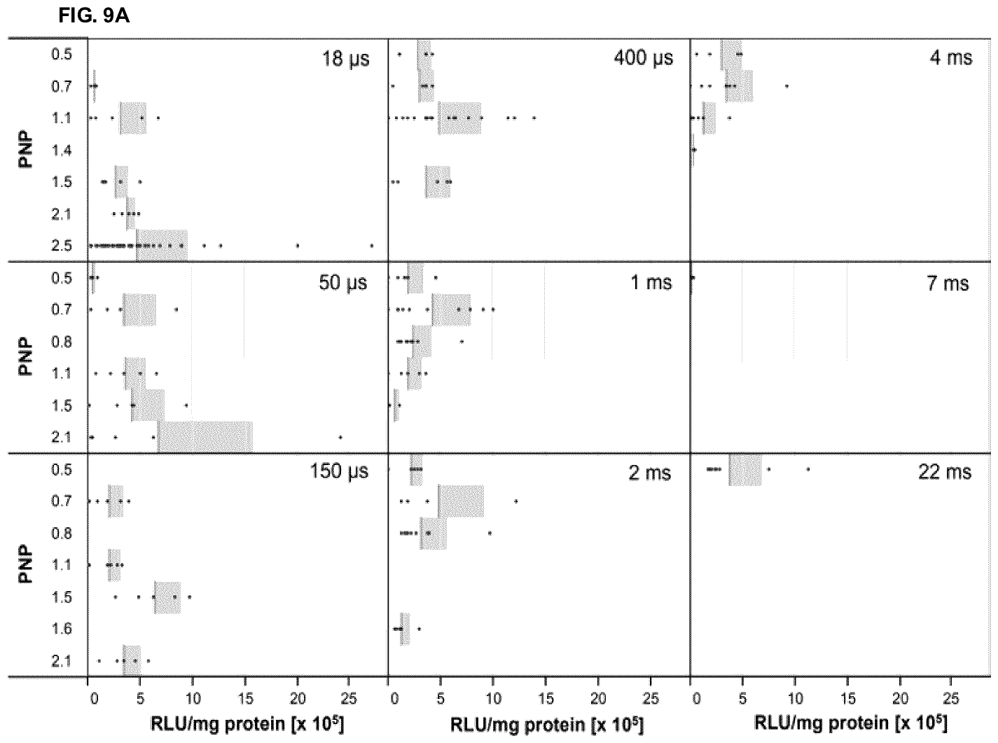

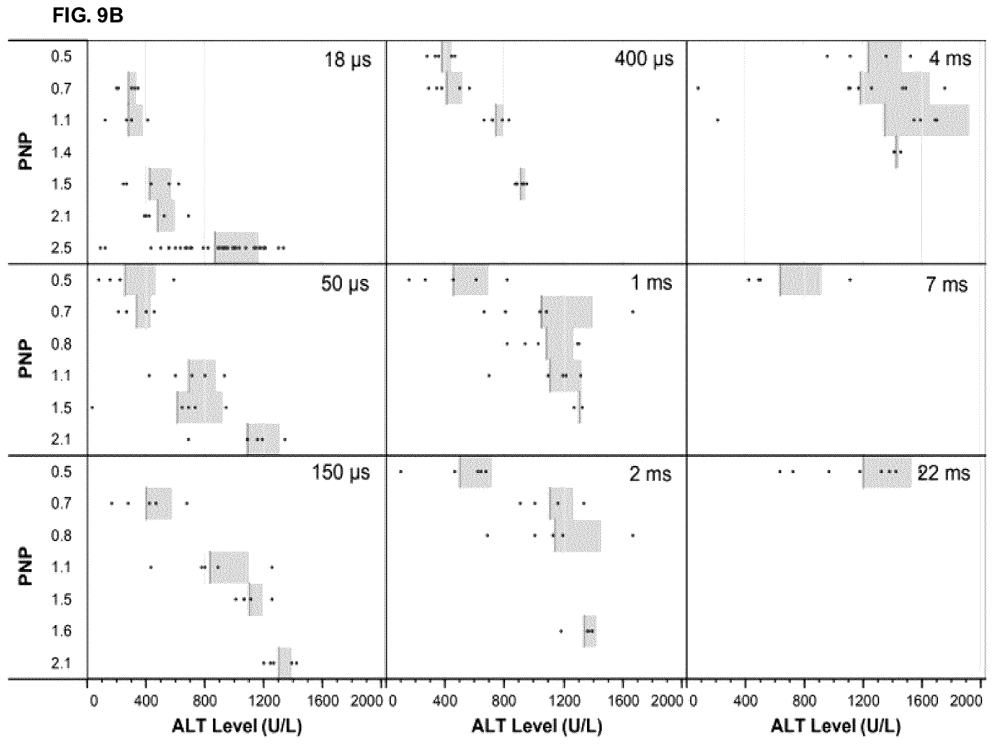

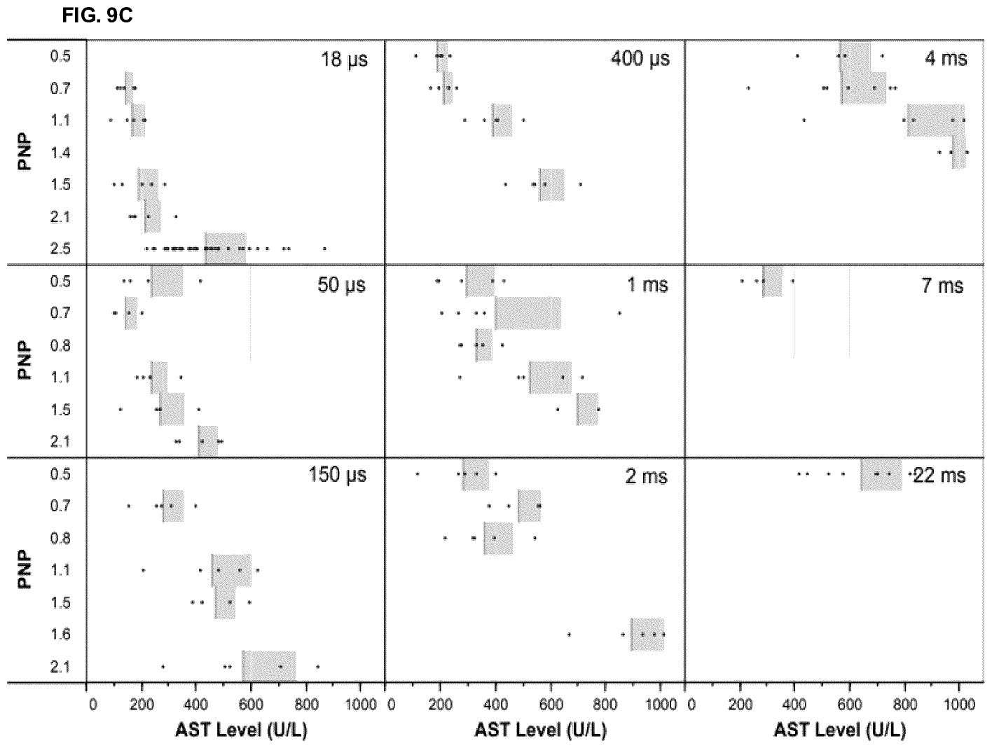

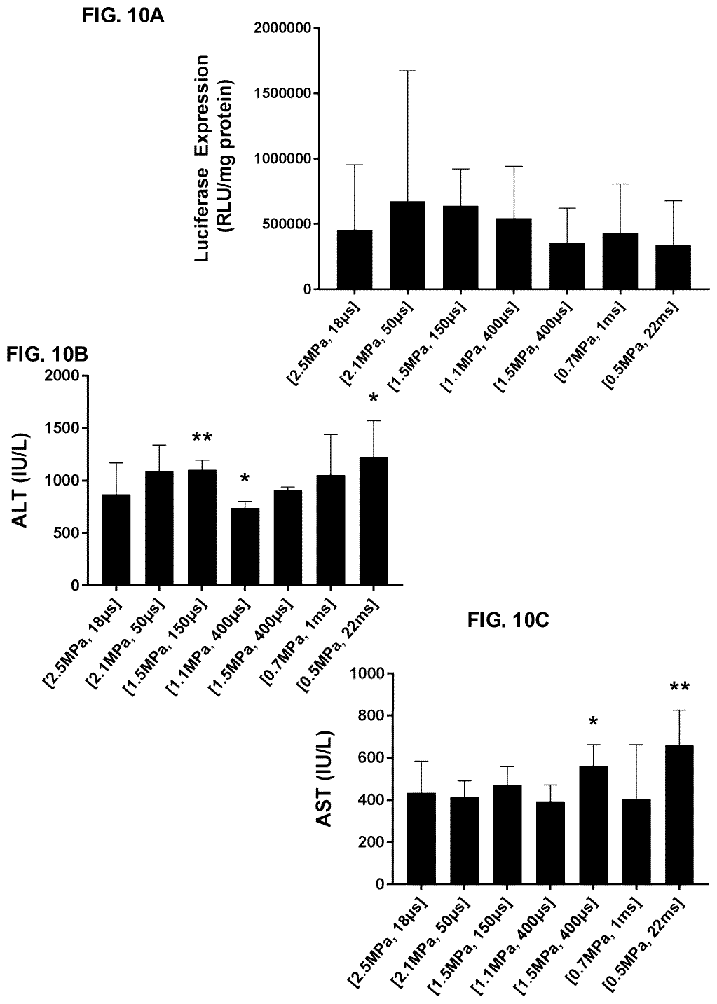

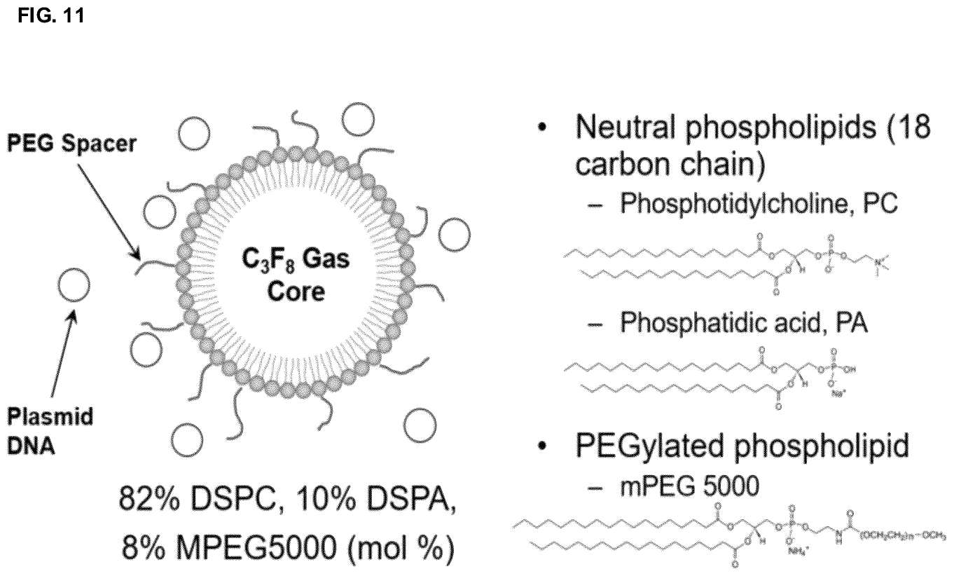

Luciferase expression results from experimental mice are summarized in FIG. 3A. Mean values and standard deviation evaluations of luciferase expression are shown in FIG. 9A. In general, at shorter pulse durations, a higher PNP value is required for efficient gene delivery. For increasingly long pulse durations, the best expression was found at decreasing PNP values. The range of acceptable pressures also appeared to decrease with increasing pulse duration. Increasing the PNP at longer pulse durations did not increase expression. This is indicated by the more rapid falloff in expression with respect to pressure at longer pulse durations. However, increasing pulse duration beyond the microsecond range at low pressures appeared to enhance gene transfer, while decreasing toxicity. Similar to UMGD experiments performed on HEK 293T cells, luciferase expression at varying US parameter combinations appears to organize close to an equi-energy curve. It was found that the majority of high-expressing conditions in this model fell on or near a curve close to 5J of total treatment energy. These high-expressing conditions were not significantly different from the positive control (FIG. 10A). Expression from various US protocols appears to be separated by three treatment energy ranges. The results from US-treated mice were pooled into three groups: mice treated with acoustic energies less than 1J, mice treated with acoustic energies between 1J and 10J, or mice treated with acoustic energies above 10J. The pooled data from each group was averaged and compared. Analysis revealed that the points that lie between the 1-10J treatment energy band were significantly greater than points that lie below the 1J energy curve or points above the 10J energy curve (P<0.005, FIG. 3B). There was no significant difference comparing the grouped points below 1J and above 10J treatment energies.

Murine Hepatotoxicity: Mouse plasma and livers were harvested 24 hours following surgery to examine any potential hepatic injury. ALT and AST enzyme levels were examined in each UMGD experimental mouse to identify conditions with increased gene expression and minimal liver tissue damage. ALT results from the panel of mouse experiments are presented in FIG. 3C and similar AST results are presented alongside in FIG. 3D. Mean values and standard deviation evaluations of the ALT and AST results are shown in FIGS. 9B, 9C. One particular condition (400 μs, 1.1 MPa) resulted in comparable transgene expression, but decreased ALT levels (FIG. 10B, 10C). It can be observed that total delivered treatment energy correlates with an increase in average ALT and AST values for each condition. Liver damage was within murine therapeutic norms for most conditions tested, though the sole condition tested lying on the 100J energy curve began to elevate above tolerable levels with an accompanying drop in expression. As a result, fewer conditions in the higher 60-100J range were tested in mice than in HEK293T experiments and the range was deemed the upper limit.

pDNA Stability after UMGD: The integrity of pGL4.13 was assessed after UMGD to determine if loss of expression at certain US conditions could be attributed to DNA instability. For conditions in which the pDNA was exposed to US, a pulse duration of 18 μs at 2.5 MPa was used for a total treatment time of 60s. This condition set was used to compare against the results of the reference condition in the in vivo experiments.

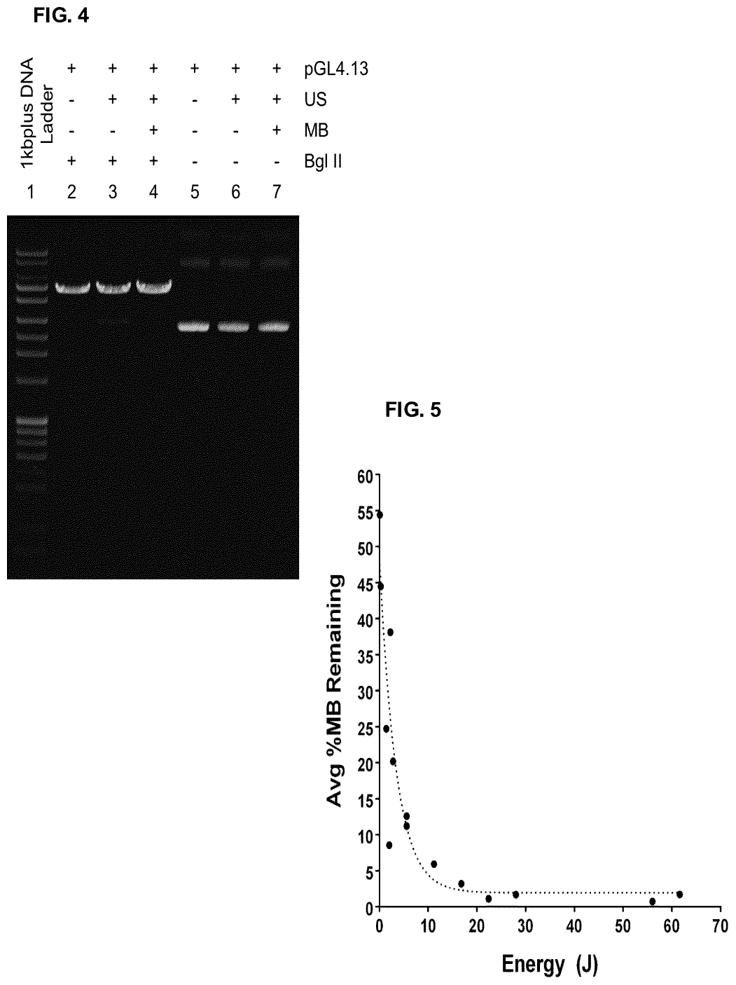

Three conditions were tested: a control untreated pDNA mixture, pDNA exposed to US without MBs, and pDNA exposed to US in the presence of MBs. The undigested and digested forms of the pDNA for each condition were compared on a gel as shown in FIG. 4. In both conditions where the pDNA was exposed to US alone and US in the presence of MBs, the integrity of pGL4.13 remained unchanged relative to the untreated pDNA. The supercoiled structure of pGL4.13 was intact, and the overall size of the pDNA remained the same for all conditions.

MB Destruction Efficiency: To allow comparison of expression results versus MB activity, endpoint studies of MB concentration were conducted using flow cytometry. Conditions were selected as a representative subset of those previously tested in HEK293T cells. It was found that once again, delivered US energy closely predicted results, although the trend was not a simple linear correlation. A comparison of treatment energy versus the extent of MB destruction is shown in FIG. 5. For treatment energies above 5J, there is a rapid drop-off in the average percent of MBs remaining after US exposure. The data is fitted by a single-decay, exponential curve.

### DISCUSSION

The potential for UMGD as means for a targeted and safer method of gene delivery has previously been shown. Other methods incorporating delivery systems such as viral vectors or adenoviral vectors are hampered by immunotoxicity or poor genetic payload, respectively.

Therapeutic administration of UMGD however, has yielded spatial and temporal control over delivery in animal experiments and is well-tolerated (Song et al., Gene Ther., 18:1006-1014, 2011; Shen et al., Gene Ther. 14:1147-1155, 2008; Sun et al., J Control Release, 182:1111-120, 2014). Prior surgical methods of performing gene delivery requires opening the cavity of the subject to access the liver. However, that procedure is invasive and not ideal for clinical translation, especially for hemophilia patients. Therefore, transcutaneous UMGD may be a possibility for minimally invasive treatment. In exchange for a safer surgical procedure however, the intensity of the acoustic wave may be attenuated via absorption by soft tissue when treating across several tissue layers. It was therefore hypothesized that a greater PNP would be required for sufficient gene delivery transcutaneously. Achieving this goal had been constrained by the upper limits of the transducer materials, US parameter boundaries, and resulting adverse bioeffects.

To overcome the barrier imposed by the transducer limits, the effects of varying US parameter pairings-pulse duration and acoustic pressure-were investigated. In previous animal study, 2.7 MPa PNP was deemed appropriate for the desired transfection effect with minimal tissue damage. To perform hepatic UMGD transcutaneously, a more powerful transducer had to be designed to meet the 2.7 MPa PNP requirement on the surface of the liver. Alternatively, whether it was possible to replicate the transfection efficiency of previous results using other PNPs and pulse durations was studied. A matrix of varying pulse durations and PNP values was generated to extend previous US parametric explorations (Song et al., Gene Ther., 18:1006-1014, 2011). When tested in vitro, increasing the pulse duration was found to result in greater GFP expression compared to shorter durations. As the pulse duration was increased, the PNP required for transfection decreased and the local GFP expression maximum occurred at progressively lower PNPs. Cell viability was maintained at 70% or greater for most conditions. A narrowing range of optimal applied pressures with maximal expression was observed as pulse duration was increased. Interestingly, it appeared the local maximums in each experimental pulse duration set clustered along a treatment energy curve upon mapping the expression results to PNP against pulse duration. Likewise, the decrease in cell viability trended towards clustering around specific energy curves. Other studies have found similar results, suggesting cell transfection and viability may correlate to ultrasound energy exposure (Keyhani et al., Pharma. Res. 18:1514-1420, 2001; Guzman et al., J Acoustical Soc. 110:588-96, 2001). Those publications report optimal energy exposures in the range of 10 to 40 J/cm², using a low frequency of 24 kHz.

The energy levels observed in the current work required to significantly sonoporate HEK 293T cells are greater than what has been previously reported by other groups. This difference may be explained by how the method chosen to calculate treatment energy, wherein the duty factor has been incorporated. In addition, Helfield et al. has suggested that at fixed frequencies, longer pulse durations are required to increase the occurrence of bubble oscillation necessary for sonoporation in vitro (PNAS 113:9983-9988, 2016). Karshafian et al. have suggested sonoporation having a dependence on pulse center frequency instead (Ultrasound in Med & Biol. 35:847-860, 2009). This may be due to different sized MBs having varied resonant frequencies that allow them to oscillate (Sun et al., IEEE Trans Ultrason Ferroelectr Freq Control 52:1981-1991, 2005). Differing driving frequencies were not texted in the current work, since the chosen 1.1 MHz driving center frequency setting allowed 100% power transfer across the H158 transducer with 90% output efficiency. Thus, it may be that only a subset of the MB population is able to undergo cavitation and contribute to sonoporation. However, the end-point MB cavitation experiments show that for nearly the entire range of power and pulse duration, and thus treatment energy, an increase led to a majority of MBs being destroyed or fragmented. This would suggest most of the MBs were initially able to oscillate at the fixed frequency and could potentially contribute to sonoporation. It is also noted that this research was carried out using a different cell line compared to other groups, which may affect the efficacy of sonoporation if membrane properties differed.

Prior studies (Noble et al., Mol Ther. 21:1687-1694, 2013; Song et al., Gene Ther., 18:1006-1014, 2011; Shen et al., Gene Ther. 14:1147-1155, 2008; Fan et al., J Control Release 170:401-413, 2013; Fan et al., Ultrasound Med Biol. 40:1260-1272, 2014) demonstrated that significant enhancement of UMGD efficiency resulted from membrane sonoporation by cavitation of the MBs upon US exposure. The efficiency is controlled mainly by US parameters and the characteristics of MBs. At low pressure such as 0.06 MPa, stable cavitation of MBs occur, whereas at pressure above 0.4 MPa, inertial cavitation of MBs can be induced and significantly enhanced gene transfection. In previous mouse studies, the in vivo gene transfer efficiency increased with increasing pressure and achieved a plateau at ≥2.5 MPa (1.1 MHz Frequency, 20 cycle pulses, 50 Hz pulse repetition frequency). Under these conditions, the total energy output from the transducer is only 1.4J due to the very short pulse duration (18 μs). However, very little enhancement was obtained at 0.4 MPa combined with 18 μs pulse length. Higher gene transfer efficiency at low pressure such as 0.4 MPa may be achievable by increasing the treatment pulse duration during the in vivo dynamic condition of US treatment. Thus, the total energy output and duty factor are increased. Increasing pulse duration can improve sonoporation based on prior in vitro studies.

The result of cell culture experiments provided a framework for translation of the US parameter matrix to small animals. Conditions above 22 ms may not be necessary for sonoporation in vivo as the optimal condition found in culture was 22 ms, 0.5 MPa. Similarly, it has been shown in previous studies that 2.7 MPa is enough to induce sonoporation with minimal tissue damage. Therefore, in the current study the upper limit of applied pressure and pulse duration were 2.5 MPa and 22 ms, respectively. 2.5 MPa, 18 μs was used as the experimental control, as used in cell culture studies for reference.

As with previous studies, a co-administration of pDNA and MBs was perfused through the portal vein while simultaneously treating the subject with US on the surface of the liver. It is believed that whole MBs cross the endothelial barrier upon permeabilization. In the case of treating hemophilia patients, where transient large pores may result in a bleeding event, pretreatment with coagulation factors may optionally be implemented to moderate bleeding during UMGD procedures. Similar to the cell experiments, optimal gene expression in mice after UMGD appears to follow a treatment energy curve. However, the range of energy curves at which optimal gene expression occurs shifted towards lower energy ranges. The US parameter pairings with optimal luciferase expression and tolerable toxicity lies between 1 and 10J. This may suggest an optimal treatment energy curve for sonoporation specific to the particular US settings and parameters defined. Within the 1 to 10J treatment energy range, ALT and AST levels begin to rise above normal ranges. As treatment energy is increased beyond this range, the total average luciferase expression decreases and instead liver enzyme levels increase well outside of normal ranges, suggestive of tissue damage. In agreement with prior work, at the longest pulse duration settings (millisecond range), increased treatment toxicity was observed for pressure settings above 1 MPa. However, the lowest pressure settings (<1 MPa) using>1 ms resulted in comparable ALT and AST values for shorter pulse duration, higher pressure settings. Overall, there is a general trend towards decreased tissue damage using lower PNP values at longer pulse durations.

The in vivo results surprisingly differed from transgene expression obtained in vitro, where the longest pulse durations provided the best gene transfer. This may be explained by the MB-mediated sonoporation having irreversible effects on membrane recovery or induction of apoptosis in vivo at both longer pulse durations and higher pressure settings without pulse train. Therefore, a compromise is made by using intermediate treatment energies to provide optimal UMGD with minimal toxicity in vivo. Lowered toxicity using longer pulse durations beyond 1 ms may be possible for treatment if a different pulse train setting is used. A pulse train would allow MBs to distribute further beyond the endothelial barrier and proximal hepatocytes before cavitating or being destroyed. Use of different pulse train settings during treatment will be considered for future studies. Although higher pressures above 1.5 MPa had not been tested at longer pulse durations, it is apparent that treatment with US above 1.5 MPa using pulse durations greater than 1 ms will decrease transfection efficiency. Using this knowledge as a guide, the US parameter pairings can be translated to larger animal models. Longer pulse durations and lower applied pressures can be selected that are equally conducive to efficient gene transfer as short pulse duration, high pressure settings, while minimizing adverse bioeffects.

The success of sonoporation in both in vivo and in vitro settings depends on MB cavitation and destruction. In the described end-point MB cavitation studies, at energies above 20J, the percentage of MBs remaining in the population is reduced to about 2%. Notable transfection occurred above 20J in UMGD of HEK 293T. This suggests MB destruction plays a role in transfection of HEK 293T cells in the experimental set-up. In mice however, MB destruction does not appear to be the sole factor in determining sonoporation. Treatment energies between 1-10J were observed to be most ideal for in vivo transfection using tested US parameter settings. Treatment energies within the 1-10J range destruct or fragment 45-90% of the MB population based on end-point cavitation results. The end-point cavitation study is not able to quantify the number of MBs that could have fragmented during US exposure. Thus, the efficacy of UMGD in vivo may depend on additional factors that are not captured in the end-point cavitation study.

One consideration that also affects microbubble behavior is the viscosity of the medium in which it is contained. The herein described in vitro studies were performed in water, which is a less viscid fluid than blood. At low applied pressures in water, MBs tend to fragment to smaller sized bubbles. However, given the higher viscosity of blood, the radial oscillations of MBs can be dampened and the occurrence of fragmentation reduced. This could increase the likelihood of and prolong multiple viable sonoporation events. As the in vivo setting is dynamic, blood flow also plays a role in translating the MBs downstream from the portal vein injection site, increasing the volume of tissue exposed to cavitating MBs. MBs can be translated by primary and secondary Bjerknes forces which also enhance treatment volume. Similar consideration can also be applied to the restricted dense space in the extravascular tissue area where MBs are in close contact with the target liver cells. These phenomena may contribute to the observation that efficient gene transfer occurred in lower energy band (1-10 J) in mice compared with higher energy band (20-100 J) in cell culture experiments. Other reasons for needing greater pressures, longer pulse durations, or energy settings in vitro have been proposed by other groups, such as difference in membrane properties of cells, MB and cell concentration, and cells being in suspension rather than adherent.

There are significant limitations to designing US transducers and protocols that will achieve efficient gene transfer in large animal models and humans due to US intensity attenuation through several tissue layers, increased treatment area/volume, and potential tissue damage via UMGD. The current study focused on defining US parameters that can (1) decrease potential damage to the tissue, and (2) decrease the requirement of high peak intensity of US for efficient delivery, thus rendering workable designs of effective US transducers within the limitation of piezo materials and further reducing potential tissue damage. The US parameter pairings between the 1 and 10 J energy curves generated optimal luciferase expression levels and tolerable toxicity. Specifically, increasing pulse duration lowered the pressure threshold required for efficient gene transfer. Thus, it was demonstrated that feasible US protocols can minimize damage to tissue and maintain efficient gene transfer, while overcoming limitations in transducer design. Lowering US peak intensity will also allow flexibility in transducer design where highly focused, narrow beams may not be necessary and can therefore increase treatment area/volumes. This is especially important and beneficial when translating to larger animal models wherein adverse bioeffects are a concern and tissue volume is much greater.

The conditions outlined as optimal in these experiments were optimized under specific US settings and transducer design, and may change depending on the design of the transducer or desired target tissue. However, the results and conclusions drawn from the very extensive exploration of US parameters in this study, especially the correlation between transfection efficiency or tissue damage and US conditions including pulse length, peak negative pressure, and energy curves have provide guidance on how to achieve successful translation of US technology from small animal experiments to large animals and human applications. These results serve as a guideline for overcoming obstacles in UMGD due to limitations of transducer design or adverse bioeffects to facilitate broader applications of US-mediated delivery.

### Example 2. Minimally Invasive Procedure for Ultrasound-Mediated Non-Viral Gene Delivery to Liver in a Porcine Model

In this example, changes in beam patterns such as focused, unfocused, or cylindrically focused beams in transducer designs are evaluated. Other changes including number and configuration of elements, or driving center frequency are also considered, as are various US parameter settings and treatment energy, which appear to play a role in UMGD bioeffects.

Significant gene transfer enhancement was realized using targeted, ultrasound (US)-mediated gene delivery (UMGD) of non-viral vectors in large animal models via an open surgery procedure; see Example 1. The goal to develop a minimally invasive treatment protocol that involves therapeutic US (tUS) across the skin for ease of clinical translation was handicapped because gene transfer efficiency was significantly reduced with transcutaneous UMGD due to US power attenuation across multiple tissue layers. Therefore, different US transducers and parameters were explored to overcome power loss while maintaining gene transfer efficiency.

A balloon catheter was inserted into the pig through a minor jugular vein incision, guided to the portal vein branch of the target liver lobe, and the location confirmed with fluoroscopy. The balloon was inflated to occlude flow, and placement of the tUS transducer was determined by diagnostic US imaging using phospholipid microbubbles (MBs) as contrast agent. tUS exposure and infusion of pGL4 plasmid (encoding a luciferase reporter gene) and MBs was simultaneously initiated. tUS was delivered via H105 (an unfocused disc transducer; Sonic Concepts, Inc.), H114 (a focused 4-element array transducer; Sonic Concepts, Inc.), or two varying configurations of XDR106 (an unfocused array transducer family designed specifically for transcutaneous compound delivery in large animals).

Livers were harvested 24 hours after surgery, assayed, and spatially-mapped for analysis of gene expression. The developed method allowed minimally invasive access to focal regions of deep-tissue organs for treatment. Following the procedure, the pigs generally recovered quickly without adverse effects. In conjunction, significant gene transfer using H105 and H114 was achieved relative to control lobes without US exposure. Transcutaneous UMGD via H105 in regions of treated liver tissue yielded a 100-fold increase to control in luciferase expression, and obtained wide spatial distribution of gene transfer due to its high treatment volume. H114 resulted in a 3000-fold increase to control, significantly improving enhancement with levels comparable to those achieved in open surgery pig experiments (>104 RLU/mg protein), however treatment area was reduced. Treatment volume and high peak negative pressure (PNP) output were both required to achieving therapeutic levels by UMGD, but improvement of one parameter sacrificed enhancement of the other.

The array transducer family, generally called XDR106, was designed that surpassed the barrier to allow improved treatment volume without sacrificing PNP output and to offer optimal UMGD efficiency. Despite increasing the range of applied PNP values (3.0-12.0 MPa) and relative gene expression, ALT and AST values remained within normal ranges using both configurations of XCR106.

These results demonstrate the success of minimally invasive, interventional radiologic techniques combined with transcutaneous US treatment to significantly enhance gene transfer to targeted liver lobes in pigs. Also provided are novel US transducers which minimize power attenuation across several tissue barriers for efficient UMGD.

### Methods, Results, and Discussion

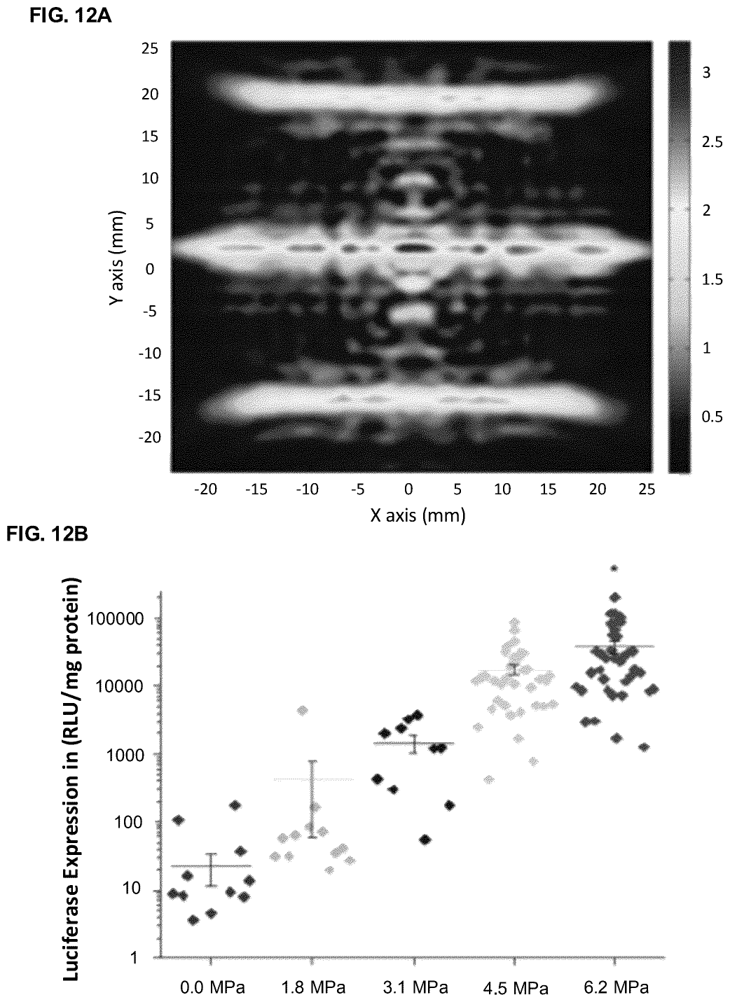

This study aimed to develop high-intensity, focused ultrasound in pigs as a model organism for targeted delivery of therapeutic compounds. Shown in FIG. 12A is a pressure profile map of the beam pattern for one of the developed focused transducers. In FIG. 12B is a plot of luciferase expression, reported as relative light units/mg protein on the y-axis, with respect to increasing PNP settings. Each dot depicts one liver tissue section.

This particular focused transducer design was able to output up to 6.2 MPa of peak negative pressure, and achieved significant gene enhancement relative to sham, or having no US applied with pDNA/MB infusion.

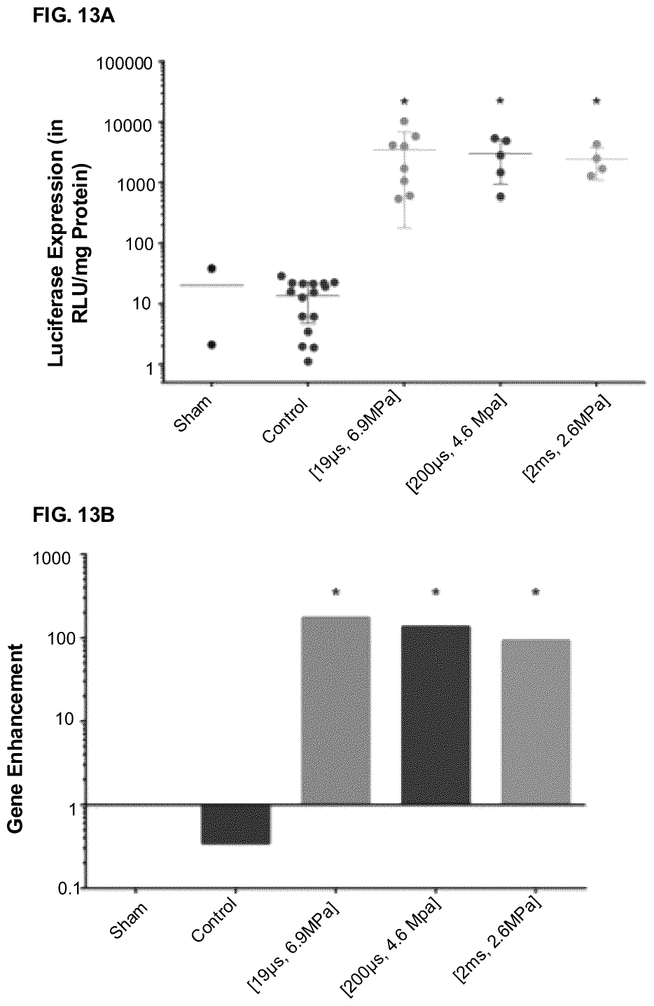

Signs of tissue damage were detected when using high PNPs, as indicated by elevated transaminase levels and tissue structure analysis. Therefore different ultrasound protocols, were investigated, simultaneous to innovative transducer development, to minimize tissue damage while maintaining efficient gene transfer. Increasing the pulse duration over ten-fold increments can help lower the acoustic threshold for microbubble cavitation, and therefore gene transfer using UMGD. FIG. 13A is a plot of average luciferase expression in individual pigs, represented as dots, in different US treatment groups. FIG. 13B shows the fold-enhancement of gene expression relative to sham. Increasing pulse duration at 200 μs or 2 ms generated equivalent gene transfer as using a 19 μs, high PNP setting, with over 100-fold in gene enhancement compared to sham. All US treated groups achieved significant gene enhancement compared to sham.

In previous studies, pig surgeries were performed using a protocol involving laparotomy. A midline incision was made to expose the surface of the liver (FIG. 14, left panel). Targeted delivery to a liver lobe was achieved by injecting a solution of pDNA and MBs into a segmental branch of the portal vein. Ultrasound was applied directly to the surface of the liver where the pDNA was injected. However, in order to translate UMGD to the clinic, a surgical procedure is needed that involves fewer or lower risks, especially if being applied in hemophilia patients. Therefore, a multimodal approach combining sonography and fluoroscopy was developed (FIG. 14, right panel). This method still allows targeting a liver lobe by accessing the jugular vein and deploying a balloon catheter into the main branch of a hepatic vein. The pDNA and MBs can still be injected through the balloon catheter, and US is then applied externally over the site of pDNA and MB injection.

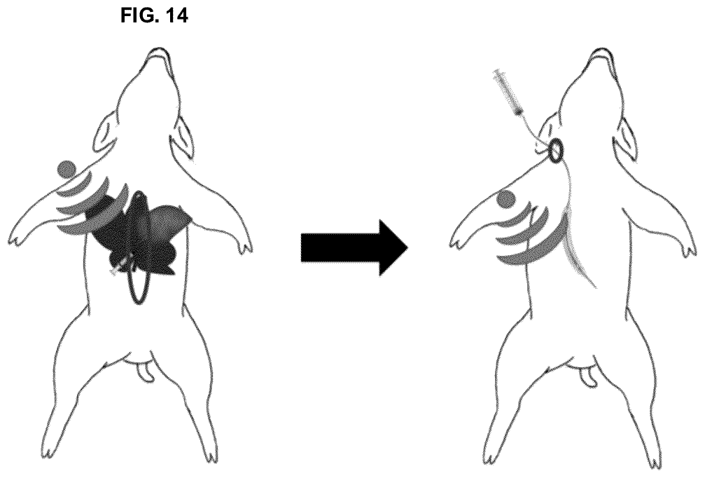

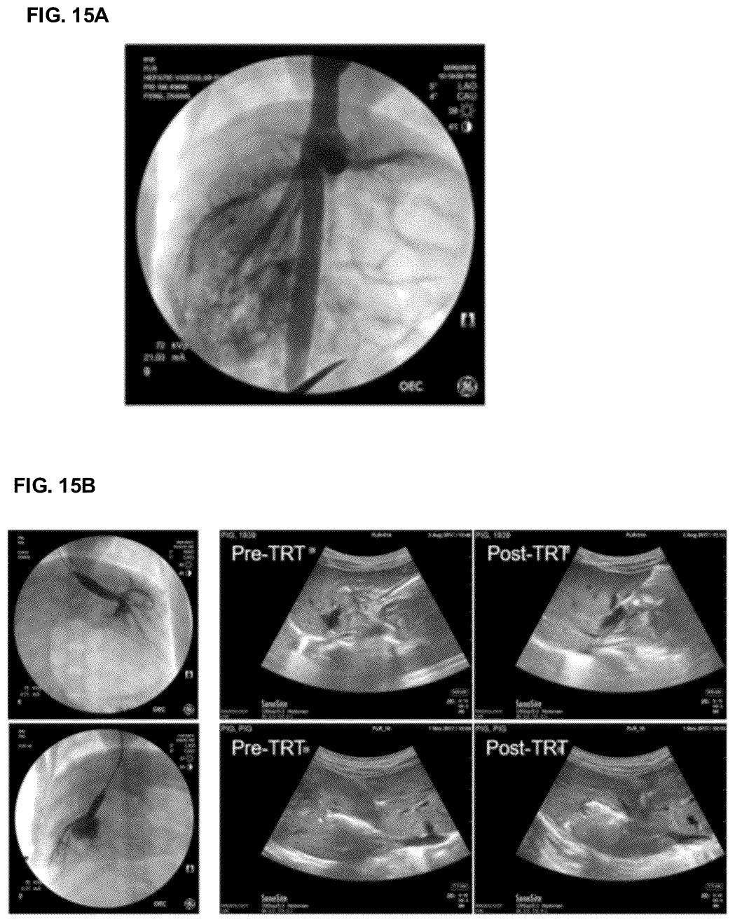

In FIG. 15A, the main hepatic branches in a pig were mapped to be used as a reference for catheter insertions in later steps. Several catheter exchanges were first performed, to access the desired hepatic vein branch and deploy a balloon catheter. The balloon is inflated to occlude blood outflow and retain the pDNA/MB solution. A solution of MBs is then infused after catheter placement, and visualized using diagnostic ultrasound before US treatment. This helps to determine the location and angle the therapeutic transducer should be placed. Whether MBs were retained or destroyed was visualize after US treatment. While the catheter placement and therapeutic transducer targeting worked well, gene transfer efficiency was reduced compared to pigs that underwent a laparotomic procedure.

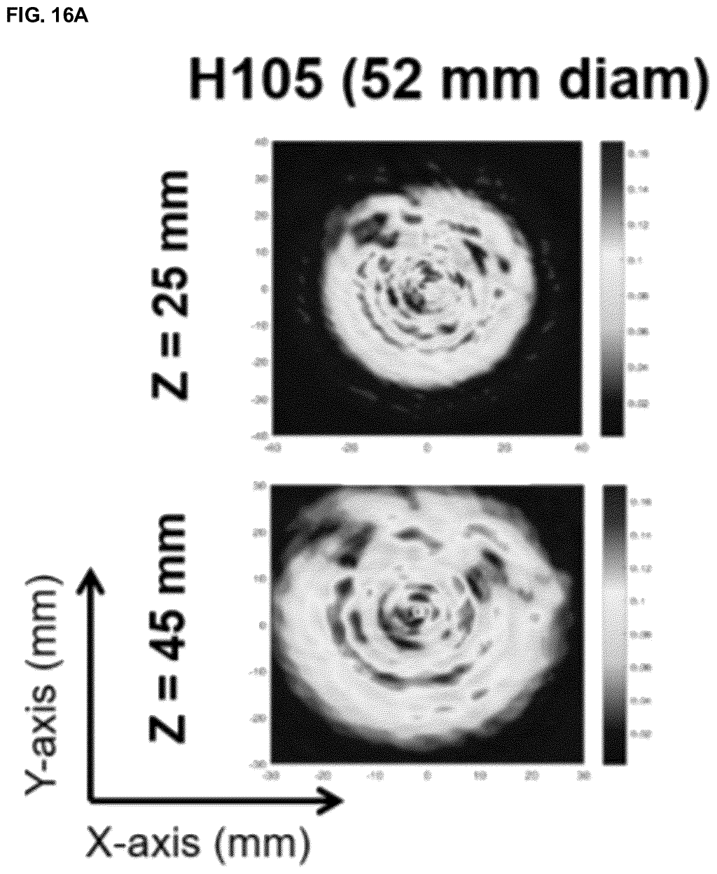

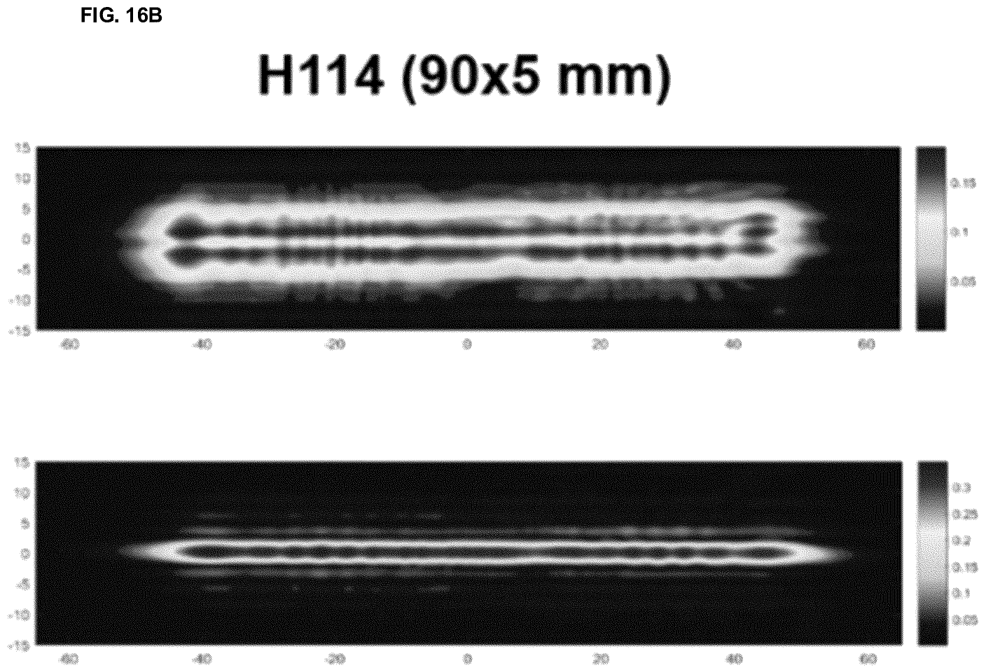

It was proposed that the main reasons for the decrease in gene transfer efficiency in transcutaneous surgeries was acoustic attenuation across several tissue barriers, and having too shallow a focal depth. Therefore, continuing with a focused transducer design, a 4-element array transducer (H114) was developed that had an extended focal depth of 45 mm that could output PNPs of greater than or equal to 12 MPa. The beam pattern of this transducer is shown in the two right-hand images (FIG. 16) at two different focus planes, 25 mm and 45 mm. The beam width is about the width of a grain of rice, and a length of 90 mm. An unfocused transducer (H105; shown on the two left-hand panels of FIG. 16) was used as a comparison to H114. These two transducers were tested to determine whether an unfocused or focused route was more effective for gene delivery in a transcutaneous application, as well as the safety profile of using each transducer.

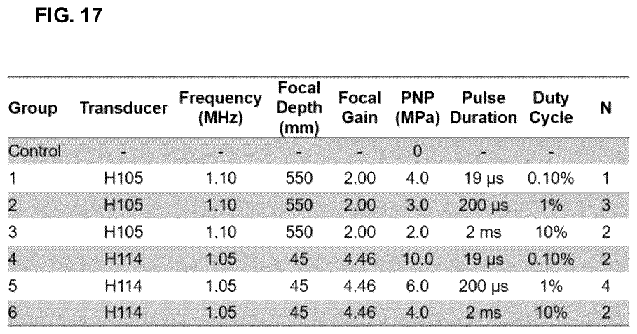

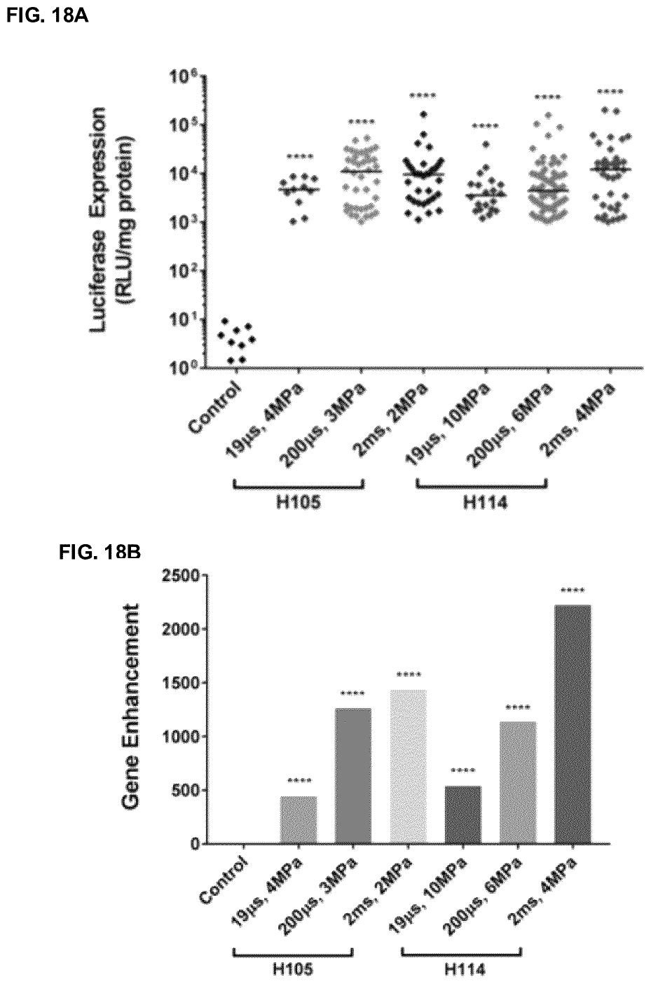

Six different US treatment protocols and one control group were used (FIG. 17). For the control group consisted of liver lobe(s) that did not have US applied across it. Three of the six US protocols used the unfocused transducer, H105, with increasing pulse duration, and decreasing PNP. The other three protocols used the focused transducer, H114, also with increasing pulse duration and decreasing PNP. N values for each protocol are as indicated.

Using either the H105 or H114 transducer achieved significant gene transfer compared to the control group. Interesting, with increasing pulse duration and decreasing PNP, w even greater expression levels were obtained-especially with H114. The highest expressing points were greater than 10⁵ RLU/mg protein using H105 and H114 at a pulse duration setting of 2 ms. This may be due to less overall tissue damage and cell death from using lower US PNPs. In general, luciferase gene transfer was enhanced by at least 500-fold using all US treatment groups.

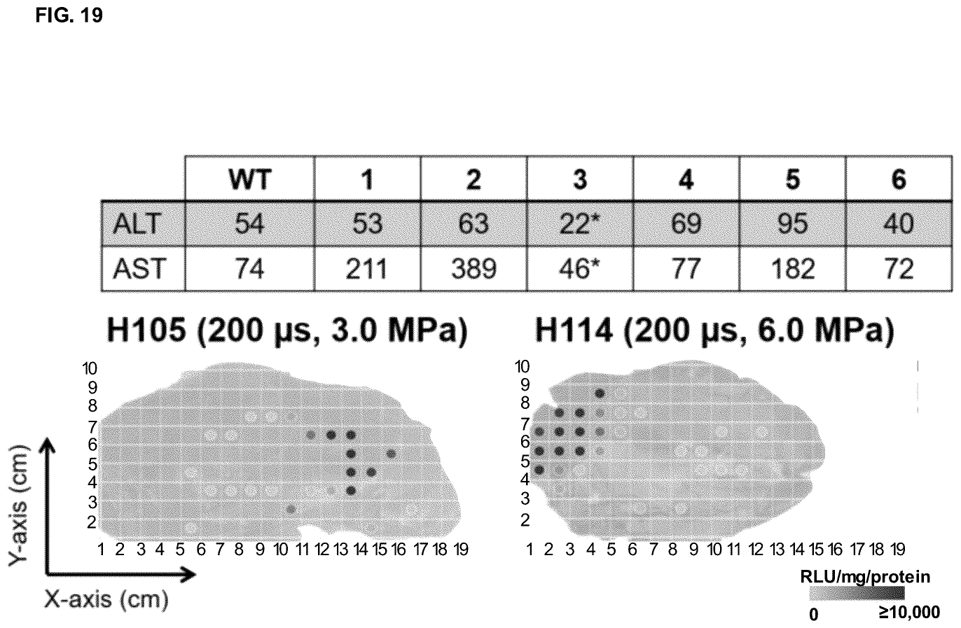

Based on transaminase levels, there is some indication that there is some tissue damage using H105, and minimal tissue damage using H114 (FIG. 19). Each value corresponds to a representative pig. Although the pig in group 3 was treated with H105, it had the lowest transaminase levels, which could be attributed to its anemic condition post-surgery. There was a spike in AST and slight elevation in ALT in the pig from group 5, but transaminase levels remained within normal levels using H114. This may be explained by H114's shortened focal length and narrow beam focus, limiting off-target bioeffects. In terms of spatial gene distribution, as shown in the Cartesian map of treated left-lateral lobes (FIG. 19, bottom panel), both H105 and H114 are capable of reaching the sites of pDNA/MB injection. Distribution is dependent on catheter placement and the user's ability to scan the transducer-particularly in the case of H114 where the beam is so narrow, it requires the user to continuously scan a copious area.

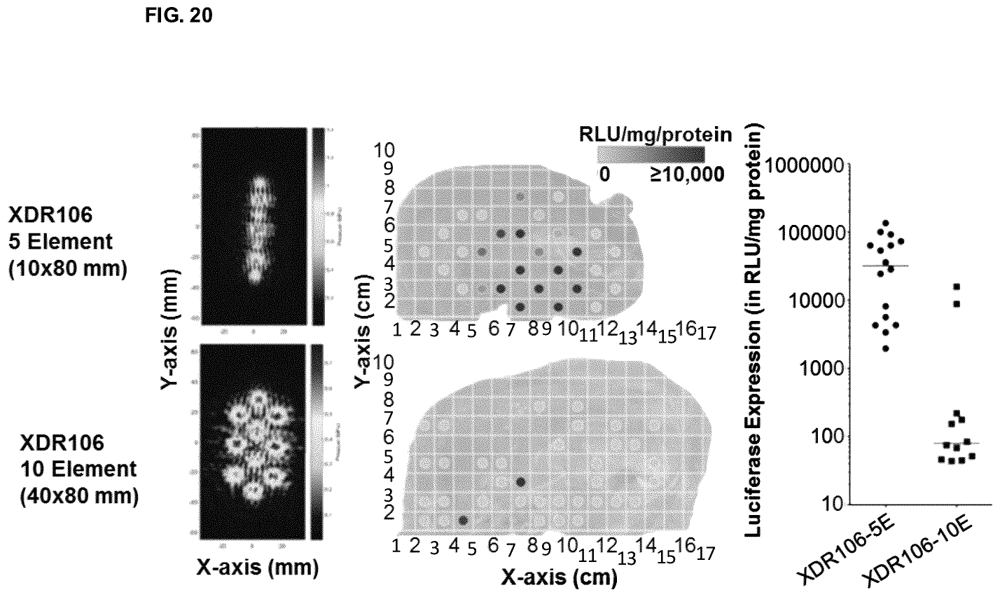

Though significant gene enhancement in liver was obtained after external US application, user variability must be improved when performing US treatment. Different transducer configurations have been designed that can leverage both the needed PNP output to circumvent tissue attenuation, as well as the treatment area for effective gene delivery. Generally, a trade-off between one of those two parameters is required if one is to be improved. However, a transducer design, generally called XDR106, has been achieved that allows both improved PNP output and increased treatment area. There are two configurations as shown by the beam plots (FIG. 20); one with 5 elements, and another with 10 elements. Gene transfer was more successful using the XDR106-5E element configuration, as opposed to the 10-element configuration (XDR106-10E), as shown by the right hand graph (FIG. 20). Without being limited to a single interpretation, this may be because the 10-element configuration does not allow the elements to directly make contact with the skin, whereas the 5-element does. In terms of spatial gene distribution, the XDR106-5 element allows the user to easily scan the transducer across the abdomen and covers a greater area as opposed to the H114 transducer, as can be seen in the Cartesian map here.

In summary, a multimodal surgical procedure has been developed that involves fluoroscopic and sonographic guidance for targeted gene delivery. Catheter placement into the left-lateral liver lobe was reliably achieved and had no major surgical complications were associated with catheter deployment. Acoustic attenuation across several tissue layers was overcome, to achieve significant gene enhancement using lower peak power densities.

### Example 3: Transcutaneous Ultrasound-Mediated Nonviral Gene Delivery to the Liver in A Porcine Model

Ultrasound (US)-mediated gene delivery (UMGD) of non-viral vectors was demonstrated in this study to be an effective method to transfer genes into the livers of large animals via a minimally invasive approach. A transhepatic venous non-viral gene delivery protocol was developed in combination with transcutaneous, therapeutic US (tUS) to facilitate significant gene transfer in pig livers. A balloon catheter was inserted into the pig hepatic veins of the target liver lobes via jugular vein access under fluoroscopic guidance. tUS exposure was continuously applied to the lobe with simultaneous infusion of pGL4 plasmid (encoding a luciferase reporter gene) and microbubbles. tUS was delivered via an unfocused, 2-element disc transducer (H105), or a novel, focused single-element transducer (H114). Supplying transcutaneous US using H114 and H105 with longer pulses and reduced acoustic pressures resulted in over 100-fold increase in luciferase activity relative to untreated lobes. This Example also showed effective UMGD by achieving focal regions of >10⁵ RLU/mg protein with minimal tissue damage, demonstrating the feasibility for clinical translation of this technique to treat patients with genetic diseases.

Introduction: The liver is a desirable target to treat a number of diseases as it is a main contributor in several metabolic pathways and production of serum proteins. Ultrasound (US)-mediated gene delivery (UMGD) has emerged as an effective gene transfer approach with great clinical relevancy and translational potential to treat various diseases (Miao & Brayman, Ultrasound-mediated gene delivery. Non-viral gene therapy (Yuan X, Ed), Intech, Rijeka, Croatia 213-242, 2011; Price et al., Mov Disord. doi: 10.1002/mds.27675, 2019; Miao et al., Hum Gene Ther. 16:893-905, 2005; Anderson et al., Gene Ther. 23:510-519, 2016; Manta et al., J Control Release. 262:170-181, 2017; Huang et al., Hum Gene Ther. 30:127-38, 2019). The technique has been applied to deliver genes and therapeutic agents to liver (Noble et al., Mol Ther. 21:1687-1694, 2013; Song et al., Mol Pharm. 9:2187-2196, 2012; Tran et al., J Control Release. 279:345-354, 2018) and various other tissues notoriously difficult to access, such as brain (Mead et al., Nano Lett. 17:3533-3542, 2017; Fan et al., J Control Release. 261:246-262, 2017; Tan et al., J Control Release. 231:86-93, 2016; Timbie et al., J Control Release. 219:61-75, 2015), bone (Bez et al., Sci Transl Med. 9, 2017; Bez et al., Mol Ther. 26:1746-1755, 2018), myocardium (Kwekkeboom et al., J Control Release. 222:18-31, 2016), and progenitor cells (Weber-Adrian et al., Gene Ther. 22:568-577, 2015). Effective UMGD relies on sonoporation events caused by exogenous cavitation nuclei such as microbubbles (MBs). MBs oscillate radially under US exposure at particular frequencies and peak-negative-pressures (PNPs) that can result in transient pores in cell membranes and opening of endothelial tight junctions. Non-viral vectors, such as naked plasmid DNA (pDNA) carrying a gene of interest, diffuse across the temporarily disrupted barrier and enter the nucleus to be transcribed. Interest lies in developing US technology and minimally invasive techniques to improve non-viral UMGD to liver tissue in order to treat human diseases.

Recent studies achieved significant gene delivery enhancement in the liver using UMGD via a laparotomic procedure in small and large animal models (Song et al., Mol Pharm. 9:2187-2196, 2012; 8, Shen et al., Gene Ther. 15:1147-1155, 2008; Song et al., Gene Ther. 18:1006-101418,-20 2011; Noble-Vranish et al., Mol Ther Methods Clin Dev. 10:179-188, 2018). Furthermore, gene transfection efficiency was improved using novel US transducer designs and beam patterns, as well as US treatment safety by modifying US protocols using longer pulse durations, and lower peak negative pressures (PNP) (Tran et al., J Control Release. 279:345-354, 2018). In order to translate recent findings into a clinically relevant minimally-invasive approach, interventional radiologic procedure was developed to facilitate transcutaneous UMGD. However, gene transfer efficiency can be reduced due to the challenge of overcoming acoustic output attenuation across multiple tissue layers (Tran et al., J Control Release. 279:345-354, 2018; Zderic et al., Ultrasound Med Biol. 30:61-66, 2004). Here, the successful optimization of US transducers and protocols in combination with a minimally invasive was showed, transhepatic venous approach to deliver plasmid DNA (pDNA) into target liver lobes to overcome transcutaneous attenuation of US intensity while maintaining effective gene delivery.

### Results

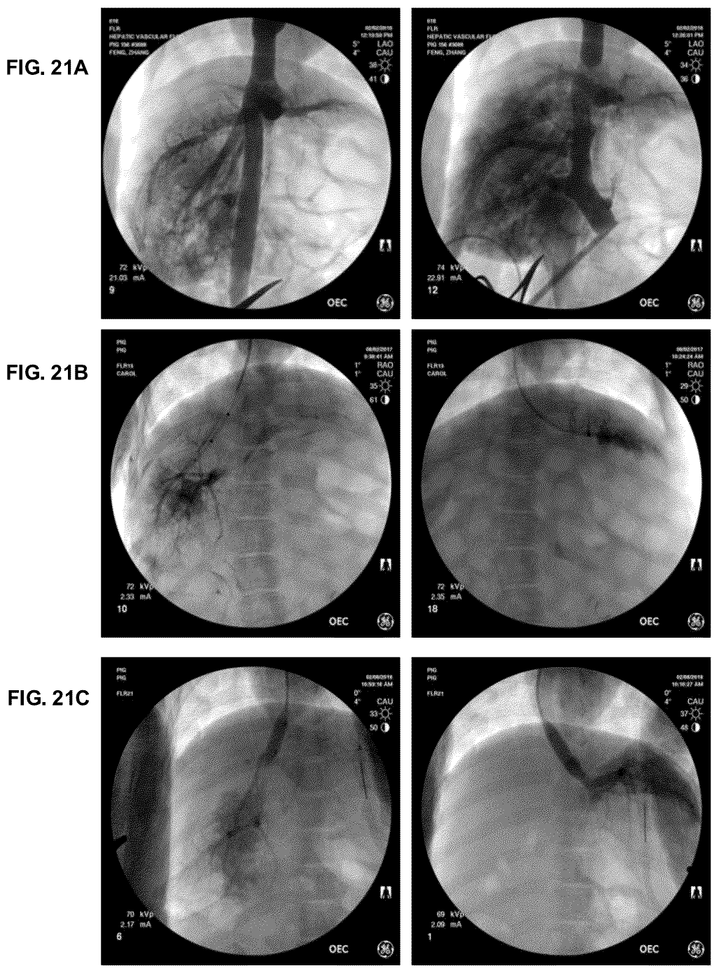

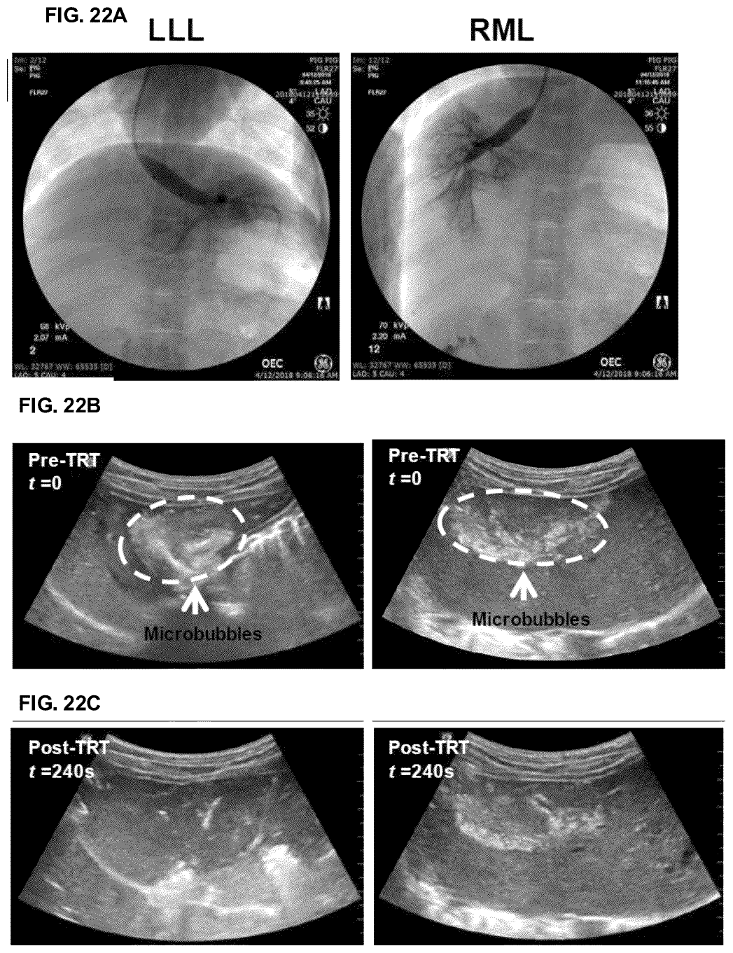

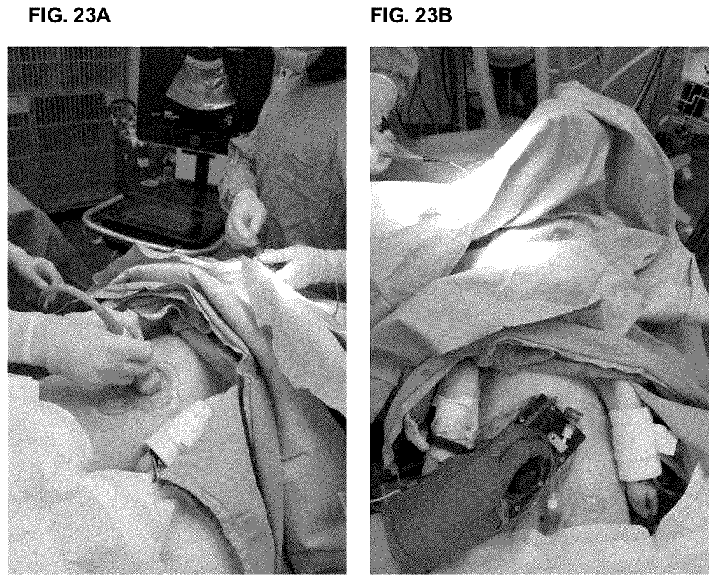

Development of a minimally invasive technique for UMGD: Via a trans-jugular-venous approach, a balloon catheter was placed in the hepatic vein of a targeted liver lobe with fluoroscopic imaging guidance. An angiography of the hepatic venous system (FIG. 21A) carried out via a terminal procedure in one pig provided reference for the targeted catheter insertion. After catheter placement, the balloon was inflated to occlude blood outflow, followed by injection of X-ray contrast agent into the liver lobe to visualize where the pDNA/MBs would distribute (FIG. 22A). Afterwards, MBs were perfused through the catheter and their retention and distribution was examined by diagnostic US imaging (FIG. 22B). Transcutaneous diagnostic imaging also helped guide the entry point of the therapeutic ultrasound (tUS) beam across the abdominal wall (FIG. 23A, 23B) and direct tUS energy towards the localized, MB/pDNA perfused lobe. tUS was applied to the targeted liver lobe for 4 minutes followed by diagnostic US imaging to visualize the retention of MBs in the vasculature (FIG. 22C). US images demonstrated MBs distributed in the left-lateral segment approximately 30-40 mm deep from the entry point of the US beam on the skin, 20-30 mm in the right-middle segments, and approximately 40-60 mm deep within the right lobe. The hepatic veins and their segmental branches could be consistently catheterized without major peri-procedural complications (FIG. 21B, 21C).

Development of tUS transducers for transcutaneous UMGD: Although transducers in the previous H185 series improved gene delivery in laparotomic procedures, they produced low levels of transgene expression in preliminary transcutaneous experiments. As a result, H114 was designed with an increased linear focal pressure gain of 4.46 and an increased focal depth of 45 mm. The focal length was geometrically increased by equipping a single curved element to produce PNPs≥12 MPa at the focus. An applied center frequency of 1.05 MHz was used to ensure maximal power conversion efficiency of the transducer and US wave propagation to deeper tissue sites.

The performance of H114 was compared to that of transducer H105 which had been used in prior UMGD experiments. H105 is an unfocused, dual-element apodized transducer with a planar surface that is driven at a center frequency of 1.10 MHz. Pressure field profiles of H105 and H114 are shown and compared in (FIG. 24A-24B). Relative pressure outputs across the US beam were measured at various distances using a 1 mm diameter hydrophone normalized to 1 MPa peak-pressure. The pressure field profile of H105 shows non-uniform PNP output transaxially. Due to its unfocused nature, the relative pressure distribution is nearly constant with respect to depth when comparing the two slices at 25 mm and 45 mm. However, the pressure field profile of H114 indicates transaxial uniformity of PNP output at 25 mm and 45 mm from the exit plane of the transducer. It should be noted that during transcutaneous US treatment, PNP is expected to decrease because of the presence of multiple skin and tissue layers above the liver. As assessed from the previous simulation model, the attenuated PNPs at acoustic maximum (Amax) during transcutaneous US treatment were estimated to decrease by 50% in intensity and are listed in Table 3. Reported PNPs are approximated derated values.

TABLE 3

| | | | | | | Derated | | | Duty |
|---|---|---|---|---|---|---|---|---|---|
| | | Frequency | Amax | Focal | PNP* | PNP** | Pulse | | Cycle |
| Group | Transducer | (MHz) | (mm) | Gain | (MPa) | (MPa) | Duration | | (%) |
| Control | — | — | — | — | 0 | 0 | — | | — |
| 1 | H105 | 1.10 | 35 | — | 3.1 | 2.2 | 19 | μs | 0.1 |
| 2 | H105 | 1.10 | 35 | — | 1.7 | 1.2 | 200 | μs | 1 |
| 3 | H105 | 1.10 | 35 | — | 1.2 | 0.8 | 2 | ms | 10 |
| 4 | H114 | 1.05 | 45 | 4.46 | 7.2 | 5.1 | 19 | μs | 0.1 |
| 5 | H114 | 1.05 | 45 | 4.46 | 3.5 | 2.5 | 200 | μs | 1 |
| 6 | H114 | 1.05 | 45 | 4.46 | 2.4 | 1.7 | 2 | ms | 10 |

*PNPs at acoustic maximum (Amax) from exit plane in the near field (<60 mm) for each transducer was measured by a 1 mm hydrophone in degassed water.
**Derated PNP at Amax was estimated assuming attenuation of 50% US Intensity through multiple tissue layers.

Gene transfer enhancement via transcutaneous UMGD in porcine livers estimate: Additional US protocols utilizing lower PNP settings with longer pulse durations were investigated to determine if efficient UMGD can be achieved using both a focused and unfocused transducer while reducing tissue damage (Table 3). The left-lateral or right-medial or-lateral hepatic lobe of pigs was treated and the luciferase activity levels in liver tissue were evaluated. The luciferase activity levels in UMGD-treated lobes were compared to control lobes without pDNA/MBs infusion, as verified by fluoroscopy and sonography, and without tUS treatment. The liver lobes were excised 24 hours after UMGD, then spatially mapped based on Cartesian coordinates, sampled, and assayed for level of luciferase activity.

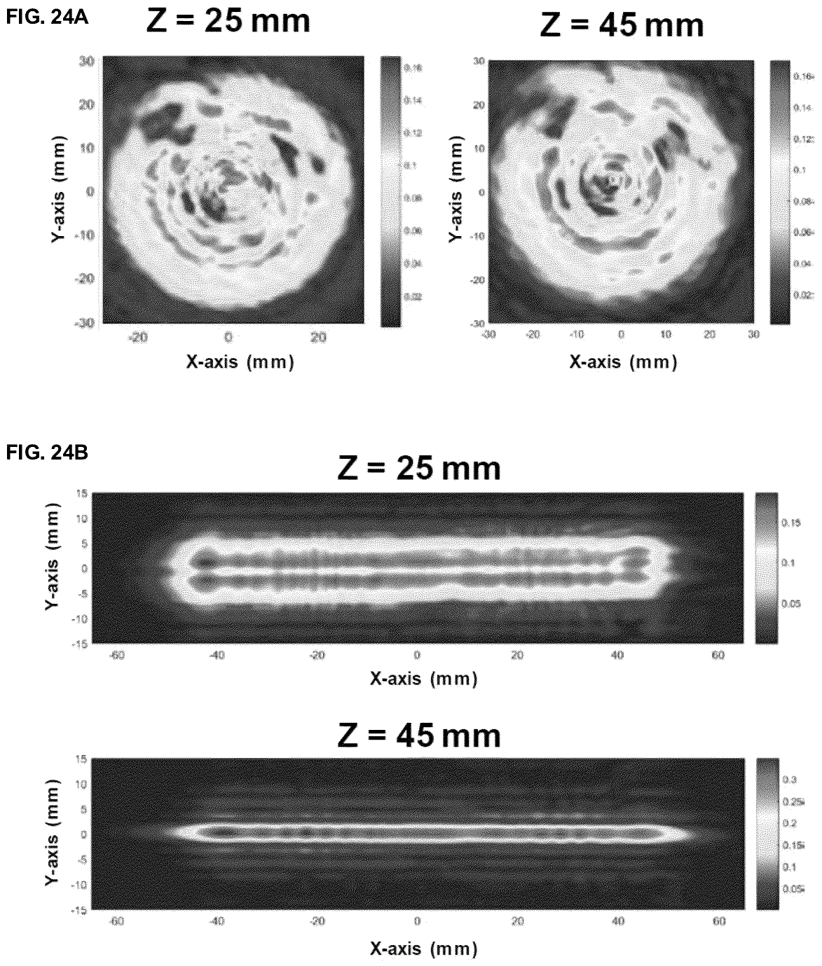

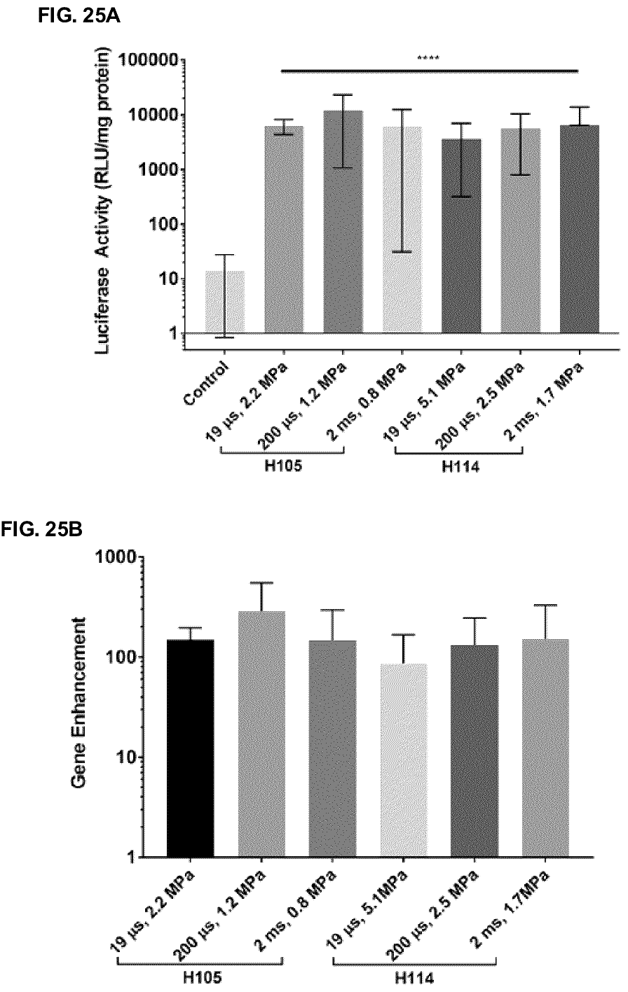

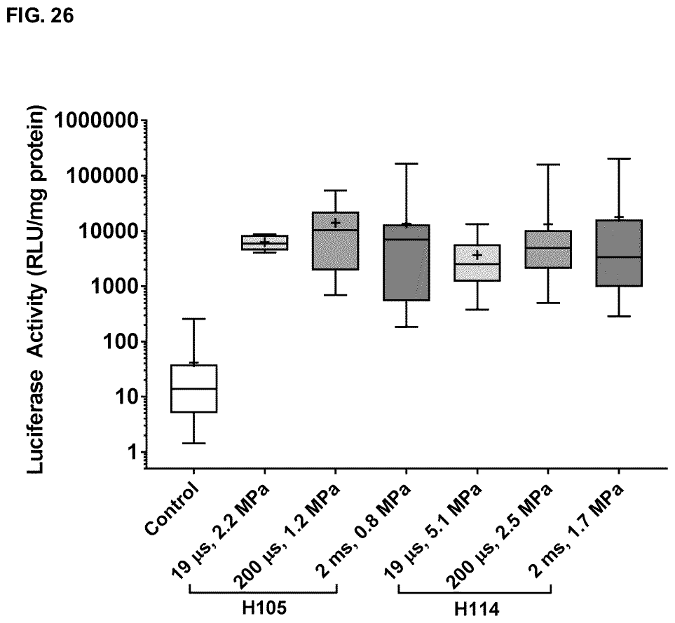

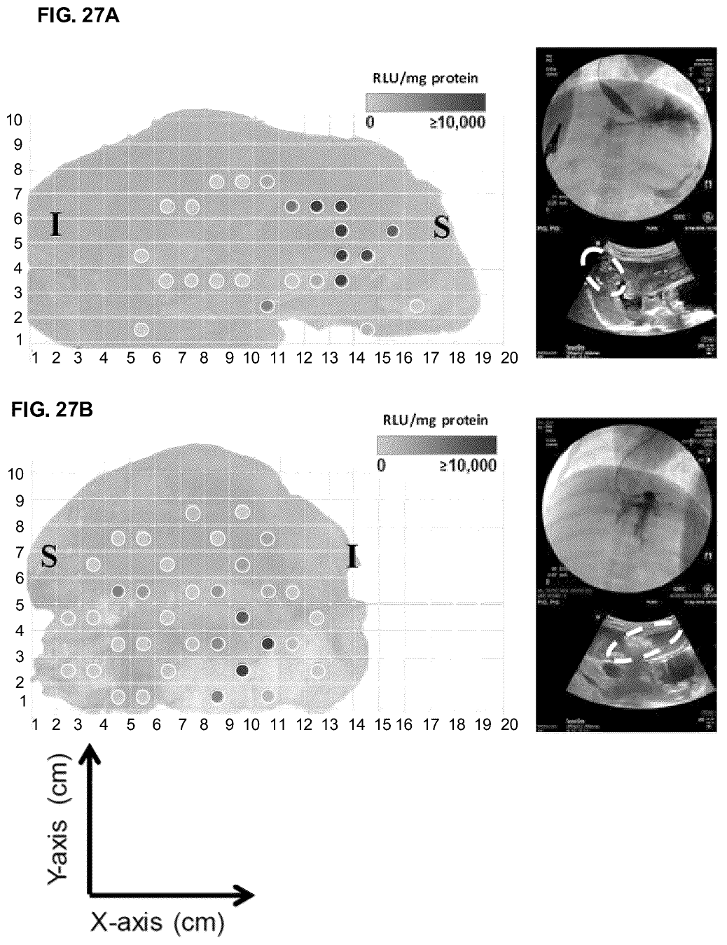

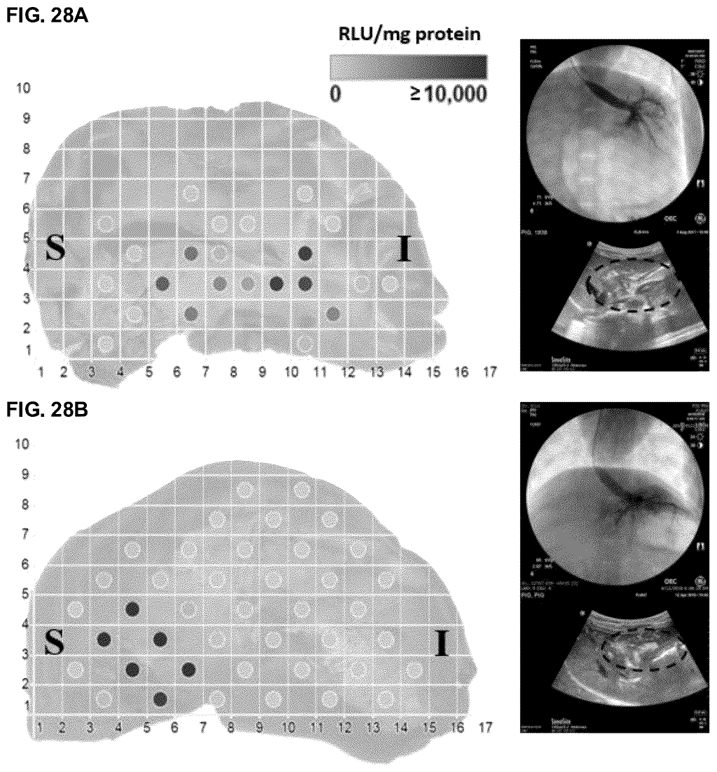

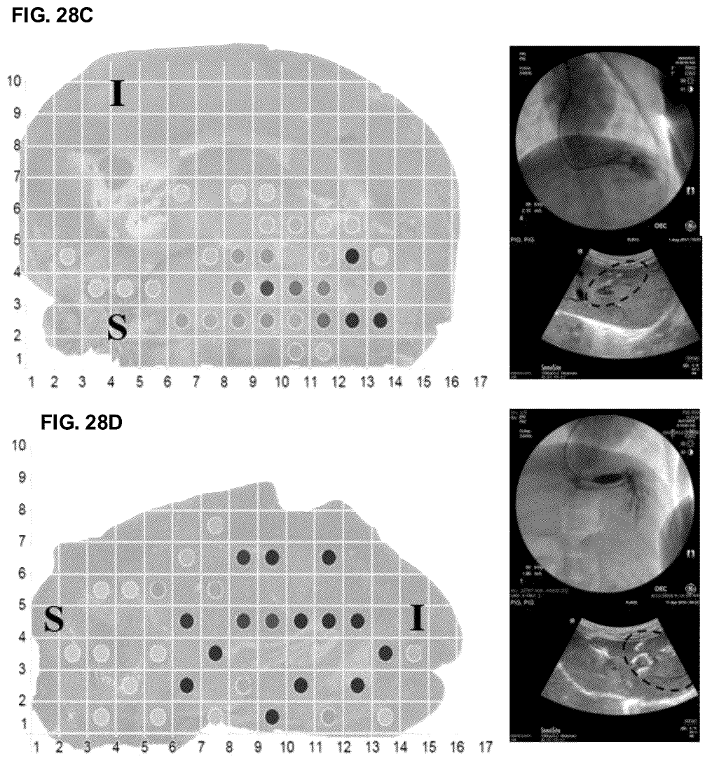

There were several points of gene transfer resulting in greater than 10,000 RLU/mg protein for each US treatment group. Sections with luciferase activity were pooled across pigs for a particular US treatment group and compared (FIG. 25A-25B). N is the sample size of pigs used, and n is the sample size of tissue sections collected for a particular US protocol amongst the treated pigs with outliers removed. The control liver lobes had luciferase activity values less than 200 RLU/mg protein (N=3, n=26). Using H114, the treatment group at 19 μs, 5.1 MPa had a maximum activity value of 13,291 RLU/mg protein, and a mean value of 3,638±3,324 RLU/mg protein (N=2, n=16); the treatment group at 200 μs, 2.5 MPa had a maximum activity value of 18,046 RLU/mg protein and a mean value of 5,537±4,748 RLU/mg protein (N=4, n=55); the treatment group at 2 ms, 1.7 MPa had a maximum activity value of 31,509 RLU/mg protein and a mean activity value of 6,361+7,435 RLU/mg protein (N=2, n=47). Using H105, the treatment group at 19 μs, 2.2 MPa had a maximum activity value of 8,709 RLU/mg protein, and a mean value of 6,260±1,944 RLU/mg protein (N=1, n=8); the treatment group at 200 μs, 1.2 MPa had a maximum activity value of 34,689 RLU/mg protein and a mean value of 12,020±10,959 RLU/mg protein (N=3, n=39); the treatment group at 2 ms, 0.8 MPa had a maximum activity value of 18,465 RLU/mg protein and a mean value of 6,168+6,137 RLU/mg protein (N=2, n=34). All treatment groups achieved significantly greater gene transfer than the control group (p-value<0.0001), and all produced over 100-fold increase in gene transfer relative to control. When comparing amongst the three treatment groups for either transducer, there was no statistically significant difference in luciferase activity levels (FIG. 25A-25B). The spread of the data including outliers is shown in (FIG. 26). Spatial expression profile maps with plotted sections assayed for luciferase activity are shown as representative maps in (FIG. 27A-27B). Spatial maps for other US protocols and transducers used are shown in (FIG. 28A-28D). The distribution of luciferase activity on the spatial expression plots corresponds well to the distribution of contrast agent in the liver lobes for all US treatment groups.

Assessment of UMGD safety profile using a highly-focused and unfocused transducer: Livers were harvested to examine the potential hepatic injury 24 hours after UMGD. Histology samples were taken from areas targeted by US treatment. There was focal hepatic injury (hemorrhage and necrosis) in several of the samples present in a pericentral distribution around the central vein in the central of the hepatic lobules (FIG. 29A-29G). The pattern of injury was limited to a few hepatic lobules with varying amounts of pericentral damage that did not involve the portal tracts or bridge between adjacent hepatic lobules. Trichrome stained liver sections from control and US-treated pigs focusing on the area of maximal injury are shown in (FIG. 29A-29G). All of the biopsies including the control had mild central venous congestion and hepatic sinusoidal dilation presumably related to the recent therapy (transient venous outflow obstruction during pDNA/MB administration). Using H114, the treatment group at 19 μs, 10 MPa presented no significant tissue damage or injury (FIG. 29B). The treatment group at 200 μs, 6 MPa presented with very small areas of pericentral necrosis involving approximately 10% of the hepatic lobules, and only 2% of the total tissue (FIG. 29C). The treatment group at 2 ms, 4 MPa also showed some pericentral necrosis in two adjacent lobules, representing 7% of the hepatic lobules and 3% of the tissue biopsy (FIG. 29D). Using H105, the treatment group at 19 μs, 2 MPa presented with focal injury in four hepatic lobules representing 10% of the total hepatic lobules examined and representing less than 3% of the biopsied tissue (FIG. 29E). The treatment group at 200 μs, 1.5 MPa presented with a greater amount of hepatic injury in which 30% of the hepatic lobules were injured representing approximately 10% of the biopsied tissue (FIG. 29F). Whereas, the treatment group at 2 ms, 1 MPa had focal hemorrhage without significant hepatic injury (FIG. 29G). The percent of injured hepatic lobules is a measure of the distribution of hepatic injury (focal or diffuse) and may be related to the US focal length of the movement of the probe during the procedure. The extent of total tissue damage is presumably related to the US conditions.

Blood was also collected 24 hours post-surgery to assess transaminase levels. No direct correlation between liver damage and US protocol or transducer could be found as two liver lobes were treated, each with a different US protocol and/or transducer, during each surgery. However, alanine transaminase (ALT) levels were calculated to be 56.9+19.5 U/L on average. The minimum ALT value obtained from UMGD-treated pigs was 22 U/L, and the maximum ALT value was 95 U/L. The median ALT value was 56 U/L. Aspartate transaminase (AST) levels were calculated to be 129+106.8 U/L on average. The minimum AST value was 46 U/L, and the maximum AST value was 389 U/L. The median AST value was 74.5 U/L.

### DISCUSSION

Procedural safety is an important factor when developing methods for clinical translation. Earlier studies had developed a minimally invasive technique to access a hepatic, region-specific area for gene delivery in large animals for hydrodynamic gene therapy (Khorsandi et al., Cancer Gene Ther. 15:225-23023,-2008; Kamimura et al., PLOS One. 9: e10720325, 2014; Fabre et al., Hum Gene Ther. 22:879-887, 2011). Thus, a multimodal procedure involving fluoroscopic and ultrasound imaging guidance was adopted for transhepatic venous, ultrasound-mediated gene delivery in the liver to minimize surgical invasiveness (Bashir et al., J Vasc Interv Radiol. 29:696-70326-28, 2018; Gebauer et al., Eur J Radiol. 69:517-522, 2009; Lee et al., Abdom Imaging. 33:555-559, 2008). Using interventional radiologic techniques under fluoroscopic guidance, the right jugular vein was accessed and the balloon catheter was placed in the left and middle hepatic veins sequentially to infuse pDNA into the liver lobes. The inflated balloon in the hepatic veins occluded blood outflow, ensuring long retention of pDNA in liver sinusoids. This offers a sufficient window of time to apply transcutaneous US to the target lobes and promote gene transfection in the liver. Catheter placement was optimized to liver lobes that can be targeted by the US beam efficiently in order to maximize gene delivery to the liver cells. No major peri-procedural complications accessing a liver lobe via the hepatic vein branch occurred during the surgeries.

In previous studies using laparotomic procedures in pigs, gene transfer could be increased using a pulse duration of 19 μs and PNP settings above 2.7 MPa using the focused H185 US transducer series (Noble-Vranish et al., Mol Ther Methods Clin Dev. 10:179-188, 2018). These focused transducers capable of higher PNPs were assessed in comparison to unfocused transducer H105 and showed improved gene delivery, but did not produce similar results in preliminary transcutaneous experiments (Noble et al., Mol Ther. 21:1687-1694, 2013). The H185 series US transducers used in laparotomy procedures were designed to have short focal depth (3-20 mm) to scan on the liver surface. Under the transcutaneous US treatment protocol, the liver was located at an increased depth under intervening skin and muscle tissue. In addition, US intensity was attenuated by multiple tissue layers and highly fibrous connective tissue surrounding porcine hepatic lobules, which may dampen the effects of MB cavitation. Therefore, a new, highly focused transducer (H114) capable of producing greater PNPs at a deeper focus was developed for transcutaneous UMGD. Considering that a focused transducer could reduce potential treatment area and volume, the focused transducer, H114, was compared with the unfocused transducer, H105, to determine whether treatment area may be a factor impacting gene transfer.

The luciferase activity assay results suggest that continuous nutation or pivoting of the focused transducer (H114) to scan the target liver lobe is able to facilitate gene transfer to a substantial fraction of a treated lobe, despite that transducer having a narrow focus approximately the width of a grain of rice. Interestingly, although the sample size was limited and direct comparison is not possible, prolonged pulse durations and lower PNP settings appeared to result in equivalent gene transfer as compared to short pulse duration, high PNP settings, similar to previous studies (Tran et al., J Control Release. 279:345-354, 2018). Both transducers were capable of producing>10⁵ RLU/mg protein using longer pulse durations, lower PNPs. The length of energy deposition over time with prolonged pulse durations may be a contributing factor. While not examined here, other studies demonstrated that as pulse duration is increased and PNP decreased, there is a higher likelihood that MBs will experience stable cavitation, enhancing mechanical influences on the cells (Mancia et al., Ultrasound Med Biol. 43:1421-1440, 2017;29 Helfield et al., Proc Natl Acad Sci USA. 113:9983-9988, 2016). The stress-induced oscillatory effect can be advanced by using targeted, pDNA-bound MBs to improve gene delivery and is planned for future studies. These results suggest the importance of both treatment volume and US protocol design for UMGD efficiency. Potential improvements in transducer design can be made by combining features of H114 and H105 to produce sufficiently high PNPs while maintaining a larger treatment area for greater coverage and delivery efficacy. One design could involve segmenting H114's single cylindrical element into a multi-channel array in order to allow variable defocusing of the US beam while maintaining high PNP outputs.

To manage the safety profile of US energy deposition, both H105 and H114 were continuously scanned across the target in an attempt to minimize focal damage from prolonged exposure as explained by Watkin et al. (Ultrasound Med Biol. 22:483-491, 1996). The advantage of using an unfocused transducer for therapy is that there is little restriction on potential treatment volume. However, H105 still has the potential to damage the hepatic tissue even in a transcutaneous setting as can be seen from the representative trichrome stains. The 200-μs setting produced the most necrosis with focal distribution following treatment. Continuous exposure to H105's large effective treatment area may possibly prolong or enhance the effects of MB cavitation, and H105 has an increased potential to damage other organs/tissues due to its unfocused nature. This can be seen from representative trichrome stains (FIG. 29E, 29F). In comparison, H114 produced a lower amount of tissue damage as detected by histology (FIG. 29B-29D). Some studies demonstrated the potential of focused US to produce necrotic lesions due to heating effects even with prolonged pulse durations and lower power settings (Damianou et al., J Acoust Soc Am. 95:1641-1649, 1994;32 Holt et al., Ultrasound Med Biol. 27:1399-1412, 2001). However, Klotz et al. showed in rats that heating effects resulting from focused US to be limited, and suggested generated heat to be contained to regions of vasculature with circulating MBs and which may actually improve vascular permeability (Klotz et al., Phys Med Biol. 55:1549-1561, 2010). Furthermore, in the current case, the heat generation is very limited due to intentionally constraining the total treatment energy by proportionally decreasing the input power as the duty factor was increased. It is most likely the necrosis seen from using H114 is a result of MB cavitation (Fan et al., J Control Release. 170:401-413, 2013; 35, Wang et al., Sci Rep. 8:3885, 2018). Furthermore, liver transaminase (ALT and AST) levels in US-treated pigs at day 1 were either normal or slightly elevated, indicating minor liver damages. The hepatic injury was primarily in pericentral distribution which may be related to the transient venous obstruction that was used during the pDNA/MB infusion. Although transient, minor liver damages may be inevitable for efficient transfection of liver cells following UMGD of plasmid vectors. These physical damages were repaired quickly after a few days as shown in previous long-term rat studies (Song et al., Mol Pharm. 9:2187-2196, 2012) in contrast to potential induction of long term side effects observed from viral vector-mediated gene transfer.

In conclusion, this study showed the feasibility of performing minimally-invasive, nonviral UMGD to the liver in a transcutaneous, clinically relevant setting. Most importantly, this study demonstrated that prolonged pulse duration allowed us to use lower acoustic pressures for efficient gene delivery while applying transcutaneous US across several tissue layers. With these successes, significant limitations were overcame in scaling-up including acoustic attenuation through multiple tissue layers, increased treatment area/volume, and potential tissue damage via UMGD to achieve efficient gene transfer in large animals. Ongoing work continues to explore delivery of therapeutic pDNA via transcutaneous UMGD in large animal models, which may pave the way towards translating the technology of minimally-invasive, nonviral UMGD to the clinic.

### Materials and Methods

Animal use protocol. All procedures were performed according to the guidelines for animal care of both the National Institutes of Health and Seattle Children's Research Institute (SCRI), with protocol approval of Institutional Animal Care and Use Committees of both SCRI and the University of Washington. S.P.F. derived Yorkshire hybrid swine (13-24 kg) were obtained from S&S Farms (Ramona, CA, USA). The swine were acclimated for at least four days prior to treatment.

Plasmid and MB preparation. The luciferase reporter plasmid pGL4.13 [luc2/SV40] (Promega, Madison, WI, USA) was produced by GenScript Inc. (Piscataway, NJ, USA) according to standard industry techniques. Preparation of RN18 MBs was described previously by Sun et al. (J Control Release. 182:111-120, 2014). Briefly, the MB shells were comprised of lipids at a 82:10:8 molar ratio of 1, 2-distearoyl-sn-glycero-3-phosphocholine (DSPC), 1,2-distearoyl-sn-glycero-3-phosphate (DSPA), and N-(carbonylmethoxypolyethyleneglycol 5000-DSPE) (Avanti®) Polar Lipids, Alabaster, AL, USA). The lipids were reconstituted and gas-exchange was performed with the headspace filled with octafluoropropane gas (American Gasp Group, Toledo, OH, USA). MB size and concentration were measured using the qNano with a NP2000 membrane and calibrated with CPC1000 particles (Izon Science, Christchurch, NZ) and analyzed using the accompanying Izon V3.3 software. Before use, MBs were activated by vigorous agitation for 45 seconds using a Vialmix™ (Lantheus Medical Imaging, N. Billerica, MA, USA), yielding an average concentration of 2-5×10⁹ MBs/mL.

US transducers and systems. The US system was previously described by Noble et al. (Mol Ther. 21:1687-1694, 2013). In this system, a laptop was used to control the signal-generating amplifiers (Model: RFG-1500BB, JJ&A Instruments, Duvall, WA, USA and Model: RPR-4000-HP, Ritec, Inc., Warwick, RI, USA) capable of producing up to 1.5 KW and 15 KW of electrical power respectively via a custom serial interface (Sonic Concepts, Bothell, WA, USA). The combined pulse generator and radio-frequency amplifier was connected to an impedance matching network to minimize reflections and maximize power transfer. The matching network is subsequently connected to either a dual-element, 57 mm diameter, unfocused disc transducer (Model H105, center frequency=1.10 MHz, Sonic Concepts, Inc.), or a single element, focused transducer (Model H114, center frequency=1.05 MHZ, Sonic Concepts, Inc.). The focus of H114 lies at 45 mm from the exit plane with a focal pressure gain of 4.46. Spatial average PNPs used with H105 ranged from 1.2-3.1 MPa. Spatial average PNPs used with H114 ranged from 2.4-7.2 MPa after including the focal gain. Derated PNP at acoustic maximum (Amax) (Table 3) was estimated assuming attenuation of 50% US intensity through multiple tissue layers. US was delivered using a 50 Hz pulse repetition frequency for 4 minutes of total US exposure.

Porcine surgery. All procedures were performed according to the guidelines for animal care of both the National Institutes of Health, Seattle Children's Research Institute (SCRI), and the University of Washington, with protocol approval of the Institutional Animal Care and Use Committee. The surgeons and veterinary staff were blinded to US protocol allocation for each pig. However, the tUS operators were not blinded as this was not possible. After administration of preanesthetic (acepromazine: 1.1 mg/kg) and induction of general anesthesia (ketamine: 33 mg/kg, glycopyrrolate: 0.005 mg/kg), each pig (15-18 kg) was placed in supine position and maintained in a stable anesthetic plane with isoflurane. The abdomen was shaved before positioning to allow better coupling between the transducer and transmission gel. The pig was draped in a sterile fashion. A micropuncture introducer set (Cook Medical, Bloomington, IN, USA) was used to access the right jugular vein under US imaging guidance (X-Porte, FUJIFILM Sonosite, Bothell, WA, USA). An 8Fr sheath (Boston Scientific) was then placed into the vein and sutured in place. A NIH angiographic catheter (Cook Medical) was placed through the sheath to cross the right atrium of the heart and access the inferior vena cava. Fluoroscopy (OEC 9900 Elite C-arm X, GE Healthcare, Little Chalfont, UK) was used to verify the placement of the angiographic catheter. The angiographic catheter was positioned to the desired target liver lobe via a hepatic vein branch and a Back-Up Meier C-Tip guidewire (Boston Scientific) was inserted for introducing a balloon catheter (20.0 mm length and 12.0 mm diameter). X-ray contrast agent (Visipaque, GE Healthcare) was injected to verify the position of the catheter. The balloon was inflated to occlude the blood outflow from the hepatic venous branch. A pGL4/MB solution (2 mL/kg solution containing: 0.67 mg/kg pGL4, 0.1 mL/kg MBs, 0.2 mL/kg 50% glucose, and PBS to total weight-based volume) was injected to the occluded hepatic region. Transcutaneous tUS was delivered simultaneously to the injected lobe for four minutes. The MB distribution in the target liver lobe was visualized by ultrasound imaging using a C60xp transducer (bandwidth=5-2 MHZ) connected to the SonoSite X-Porte before and after US treatment. This allowed us to verify the location of pGL4/MB solution and the required direction of US energy via the therapeutic transducer before treatment, as well as to determine whether MB destruction occurred after treatment. After the ultrasound treatment, catheters were removed and the incision was closed using sutures. The pigs were allowed to recover. Post-operative systemic analgesics were administered (ketoprofen: 2 mg/kg, buprenorphine: 0.02 mg/kg). Physiological parameters including heart rate, oxygen saturation (SpO2), body temperature, and systolic and diastolic blood pressure for the pig were monitored throughout the surgery by a licensed veterinary technician. After 24 hours, the pigs were sacrificed, and the treated and control lobes were harvested for sectioning and processing for luciferase expression. Blood and tissue samples were collected for liver enzyme and histological analysis.

Hepatic venography. Under anesthesia (acepromazine: 1.1 mg/kg, glycopyrrolate: 0.005 mg/kg, 3% isoflurane), a balloon catheter (Boston Scientific, Marlborough, MA) is threaded into the jugular vein and the inferior/caudal vena cava to a level cranial to the liver of the pig. A laparotomy was performed to expose the main portal vein. The portal vein was then cannulated to allow a guidewire to be placed, over which an 8F sheath was inserted into the vessel. The main portal vein was then ligated to secure the sheath. 60,000 units of heparin saline was perfused via the inferior vena cava (IVC) caudal to the liver to flush the blood from the hepatic venous system. The balloon catheter was then inflated to occlude the IVC cranial to the liver. The IVC caudal to the liver was ligated and the body wall closed. X-ray contrast agent (Visipaque, GE Healthcare) in saline was infused into the IVC and hepatic venous system via the balloon catheter. A venogram of the hepatic venous system was then obtained using an OEC 9900 Elite C-arm X-ray machine (GE Healthcare) and the pig was euthanized thereafter.

Luciferase gene expression analysis. All liver lobes were resected following sacrifice of the pig 24 hours after the procedure when the pGL4 luciferase expression reaches the peak level as experimentally determined before. The control lobe is defined as lobes without having directly been injected with the pGL4/MB solution and not exposed to tUS. Treated and control lobes were spatially mapped and sectioned into smaller pieces, and some samples were assayed for luciferase expression as previously described (Noble-Vranish et al., Mol Ther Methods Clin Dev. 10:179-188, 2018). Briefly, supernatant was collected from homogenate that underwent several freeze-thaw cycles and its luciferase activity was analyzed using a commercially available kit (Luciferase Assay System, Promega, Madison, WI) and measured by a luminometer (Victor 3: Perkin Elmer, Wellesley, MA). Luciferase expression was normalized to total protein content, measured by BCA assay kit (Bio-Rad, Hercules, CA), and reported as relative light units (RLU)/mg protein.

Blood analysis. To detect liver damage after treatment, blood samples were collected before euthanasia and sent to a commercial veterinary diagnostic laboratory (Phoenix Central Laboratory, Mukilteo, WA) for a complete blood count and a chemistry panel including alanine (ALT) and aspartate transaminase (AST) values.

Histological analysis. Treated and control liver biopsies were fixed in 10% neutral buffered formalin, then processed and embedded in paraffin. Routine hematoxylin and eosin and trichrome-stained slides were made to examine any liver damage.

Statistical analysis. All data are presented as means with standard deviation or medians with upper and lower quartiles as well as maximums and minimums. Tissue sections were selected from regions that were known to have MBs/pDNA distribution as indicated by corresponding fluoroscopy images. Additional sections were collected beyond the area of apparent MB/pDNA distribution to include analysis of regions without US treatment. Control liver lobes were also assayed for luciferase activity. An adequate number of tissue sections is believed to have been collected to cover analysis of roughly 50% or more of the liver lobe. Statistical methods were not used to predetermine sample size. Data gathered from multiple liver lobes treated with the same US protocol and transducer from different pigs were pooled. To differentiate between treated and non-treated tissue sections, a maximum to minimum luciferase activity ratio was calculated for 3 control (no MBs+no US treatment) liver lobes and the ratios then averaged to provide a background threshold ratio. A minimum luciferase activity value above zero was used, and the average background threshold ratio was determined to be approximately 100. A max-to-min ratio was calculated for each tissue section for an individual, treated liver lobe, and the section was determined to be treated if the ratio surpassed the threshold ratio. Therefore, several tissue sections were excluded from analysis due to having zero luciferase activity for control lobes or were below the max-to-min ratio for treated lobes. The minimum luciferase activity value within the individual liver lobe being analyzed was used as the denominator to calculate the max-to-min ratio, and this differed across individual liver lobes. Outliers were identified and removed from the pooled data using the ROUT method from a statistical package (GraphPad Prism 7, Prism Inc., Reston, VA, USA) with Q=1%. The data set was tested for Gaussian distribution using the D'Agostino-Pearson omnibus normality test with a confidence interval set to 95%. The data sets did not pass the normality test. Therefore, a Kruskal-Wallis single factor Analysis of Variance (ANOVA) was performed using GraphPad to compare the means of each group to the control and correcting for multiple comparisons using Dunn's test. If statistical difference was found, a Tukey test was also performed post-hoc for multiple pairwise comparisons. P-values of less than 0.05 were considered to be statistically significant.

### Example 4: Ultrasound-Targeted Microbubble Destruction Mediated FVIII Plasmid Delivery for hemophilia A gene therapy

Ultrasound (US)-targeted microbubble (MB) destruction (UTMD) has been applied for gene delivery as a promising non-viral physical strategy. As described herein, an US/MB mediated gene delivery (UMGD) system has been successfully developed, which significantly enhanced luciferase reporter gene transfer efficiency in mouse and rat models. In this study, gene delivery of a hepatocyte-specific human-factor VIII (FVIII) plasmid mediated by US/MB was further explored in the livers of hemophilia (HA) mice. On day 1 after treatment, FVIII activities in plasma showed significant enhancement, reaching up to 100% of normal levels in plasma. The therapeutic levels persisted until day 28 post-treatment with immunomodulation. Furthermore, phenotypic correction of treated mice was significantly improved compared with those of untreated HA mice. Evaluation of transaminase values and histology indicated that transient liver damages following treatment recovered within 14-30 days. Phenotypic correction of hemophilia A mice was examined by tail clip assay. Blood loss of US/MB treated mice was significantly reduced compared with naive HA mice. All these data demonstrated that US/MB can significantly enhance gene transfer of therapeutic FVIII and has high potential to improve treatment for hemophilia.

Introduction: Hemophilia A is an X-linked recessive bleeding disorder that caused by a deficiency of blood clotting factor VIII. According to the classification based on FVIII levels, individuals with less than 1% of normal activity are severe hemophilia A, with 1-5% are moderate, and with 5-40% are mild. Conventional treatment involves the replacement therapy of FVIII by intravenous infusion of plasma-derived or recombinant FVIII concentrates, which has faced challenges including the expense, the inconvenience of administration, and availability of safe products. Gene therapy as an alternative approach for hemophilia treatment has drawn significant interest, as even a continuous modest increase of FVIII levels (>1%) can result in dramatic improvement of severe bleeding phenotype (Ljung, Thrombosis and Haemostasis 82:525-530, 1999; Lofqvist, et al., Journal of internal medicine 241:395-400, 1997; Franchini et al., Journal of thrombosis and Haemostasis: JTH 8:421-432, 2010). Besides the wide therapeutic window, it also benefits from some other features of hemophilia A disease, including a wide variety of target tissues for FVIII biosynthesis (Elder et al., Genomics 16:374-379, 1993; Hollestelle et al., Thrombosis and Haemostasis 86:855-861, 2001; Wion et al., Nature 317:726-729, 1985; Rall et al., Lancet 1:44, 1985), excellent hemophilia animal models such as mouse (Bi et al., Nature genetics 10:119-121, 1995; Bri et al., Thrombosis and Haemostasis 95:341-347, 2006), rat (Booth et al., Comparative medicine 60:25-30, 2010), sheep (Porada et al., Journal of thrombosis and Haemostasis: JTH 8:276-285, 2010), dog (Lozier et al., Proceedings of the National Academy of Sciences of the United States of America 99:12991-12996, 2002). In the past, a variety of gene transfer based hemophilia A therapy were reported, including viral (Hu & Lipshutz, Gene therapy 19:1166-1176, 2012; Brown et al., Blood 122, 2013; Sarkar, et al., Blood 103:1253-1260, 2004) and non-viral strategies (Roth et al., The New England journal of medicine 344:1735-1742, 2001; Kren et al., J Clin Invest 119:2086-2099, 2009; Dhadwar et al., JTH 8:2743-2750, 2010). However, further clinical applications were hampered by vector immunogenicity (Mingozz et al., Blood 122:23-36, 2013), limit of transduced target cells (Powel et al., Blood 102:2038-2045. 2003), insertional mutagenesis (Ramezani & Hawley, Blood 114:526-534, 2009), limited gene-carrying capacity or low transgene efficiency (Brown et al., Blood 122, 2013; Grieger et al., Journal of virology 79:9933-9944, 2005).

Ultrasound (US) applied in the presence of microbubbles (MBs), also called as ultrasound-targeted microbubble destruction (UTMD), has been considered as a promising non-viral gene delivery method. In vitro studies showed that US induced acoustic cavitation of MBs can transiently increase the cell membrane permeability and enhance cellular uptake of plasmid (Schlicher et al., Ultrasound Med Biol 36:677-692, 2010; Zhou et al., Journal of controlled release: official journal of the Controlled Release Society 157:103-111, 2012). For in vivo gene delivery, UTMD can also create tissue hemorrhage, allowing extravasations of molecules from the microvessels into extravascular tissue space (Bekeredjian et al., Ultrasound Med Biol 33:1592-1598, 2007). US combine with MBs has been applied in vivo to deliver drug or gene into different target organ such as liver, brain, pancreas, kidney, and cornea for therapeutic treatment of tumor, cardiovascular, etc. (Suzuk et al., Journal of Controlled Release 149:36-41, 2011; Song et al., Molecular pharmaceutics 9:2187-2196, 2012; Aryal et al., Advanced drug delivery reviews 72:94-109, 2014; Deelman et al., Advanced drug delivery reviews 62:1369-1377, 2010).

A therapeutic US combined with an albumin-based US contrast agent, Optison (GE Healthcare Discovery Systems, Piscataway, NJ) was previously used for FIX plasmid gene transfer in a normal mouse model (Miao et al., Human gene therapy 16:893-905, 2005). The mixture of plasmid and Optison MBs was delivered by hepatic injection into the mouse liver which was simultaneously treated with US. The results show significant enhancement of short-term FIX gene expression, preliminarily demonstrating the feasibility of applying UTMD in plasmid DNA gene therapy. Subsequently, the US system, MBs and treatment protocol, with luciferase reporter gene plasmid, was developed and optimized to further improve the gene transfer efficiency (Shen et al., Gene therapy 15:1147-1155, 2008; Song et al., Gene therapy 18:1006-1014, 2011). The following most important explorations were performed in those in vivo experiments: (1) injection routes (2) comparison of focused US, far-field unfocused US and near-field unfocused US systems (3) US parameters including peak negative pressure, pulse duration, duty factor, pulse repetition frequency (4) pulse-train US exposure regimens (5) MB types and concentrations (6) liver damages induced by US/MBs treatment. Portal vein injection showed superior to intrahepatic injection as the gene delivery route into live, with better distribution, resulting in more efficient gene transfer. Inertial acoustic cavitation mechanism was indicated by the dependency of gene transfer efficiency upon PNP as well as MB concentrations. Furthermore, US parameters and MBs were optimized, based on the correlation of gene expression and liver damages induced by acoustic cavitation, to produce maximal desired gene transgene expression with the minimum liver tissue damages. Moreover, application of pulse-train US even offered comparable or higher gene transfer efficiency with less liver damage (Song et al., Molecular pharmaceutics 9:2187-2196, 2012; Song et al., Gene therapy 18:1006-1014, 2011). Based on these studies, the US/MBs system has been scaled up from mouse model to rat model, and forward towards large animal models (Song et al., Molecular pharmaceutics 9:2187-2196, 2012; Noble et al., Molecular therapy: the journal of the American Society of Gene Therapy 21:1687-1694, 2013). Accordingly, novel neutral or cationic MBs developed for UMGD was explored in vitro and in vivo (Sun et al., Journal of controlled release: official journal of the Controlled Release Society 182:111-120, 2014), which implied a potential to generate more efficient UTMD system by various functional modifications.

All the previous achievements provided a platform for pursuing an efficient and safe treatment with UTMD for hemophilia A gene therapy. As a first step, US/MBs mediated gene transfer of therapeutic FVIII plasmid was explored in a hemophilia mouse model. The successful results paved the way to deliver FVIII plasmids into large animals and humans.

### Method

Animals: All mice were maintained at a specific pathogen-free (SPF) vivarium in accordance with the guidelines for animal care of both National Institutes of Health and Seattle Children's Research Institute. Animal protocols were approved by the Institutional Animal Care and Use Committee of Seattle Children's Research Institute. 8-week-old C57BL/6 normal mice were purchased from Charles River Laboratories International, Inc. (Wilmington, MA, USA) and housed under SPF conditions for at least 3 days before the experiments. Hemophilia A mice in Balb/C background (HA/Balb/C) were generated by backcrossing FVIII exon 16 knockout hemophilia A mice in C57BL/6/129sv mixed genetic background with Balb/C mice for more than 10 generations. 8-12 week-old HA/Balb/C mice were used for the studies.

Plasmids and MBs: Luciferase reporter plasmid (pGL4.13 [luc2/SV40] (Promega)) was prepared by GenScript Inc. (Piscataway, NJ, USA). Reporter pGFP plasmid driven by a CMV promoter (pGFP) was prepared and amplified using Maxiprep kit (Qiagen, Germantown, MD) according to the manufacturer's protocol. A liver specific human FVIII plasmid pBS-HCRHP-FVIII/N6) carrying a hFVIII/N6 cDNA driven by the hepatic control region (HCR) and a liver-specific α1-antrypsin (HP) promoter was constructed. The hFVIII/N6 molecule contains a partial B-domain deletion leaving an N-terminal 226 amino acid stretch containing 6 intact putative asparagine-linked glycosylation sites. This modification increases in vitro and in vivo secretion of FVIII by 10-15 fold and also reduces anti-FVIII immune response (Miao et al., Blood 103:3412-3419, 2004). This modified FVIII/N6 cDNA as obtained from Steven Pipe at U. Michigan and inserted it into previously reported liver-specific gene expression vector (Miao et al., Human gene therapy 14:1297-1305, 2003). In all gene transfer experiments, 50 μg of reporter plasmid DNA or 100 μg of therapeutic plasmid DNA per mouse was mixed with 5% Vol. NuvOx MBs in 400 μl PBS containing 5% glucose. NuvOx MBs (NuvOx Pharma, Tucson, AZ) is a lipid-shelled US contrast agent composed of encapsulated octafluoropropane gas, with a concentration of ˜1×10¹⁰ MBs/ml and a mean diameter of ˜2 μm. MBs were reconstituted by shaking the vial for 45 sec with a Vialmix agitator (Lantheus Medical Imaging, North Billerica, MA) immediately right before use.

US system: The US system, as described previously (Song et al., Molecular pharmaceutics 9:2187-2196, 2012; Song et al., Gene therapy 18:1006-1014, 2011), was composed of a separate pulse generator and a high power radio frequency pulse amplifier, which is capable of generating up to 1000 W of electrical power into a 50 0 load (model RFG-1000; JJ&A Instruments, Duvall, WA). A customized single-element, 1.1 MHZ, 16 mm-diameter transducer (model H158A; Sonic Concepts, Bothell, WA) driven by the US system was used to directly apply acoustic pressure to the surgically exposed liver surface via enhanced coupling with sterile US transmission gel.

In vivo reporter gene transfer: C57BL/6 normal mice were anesthetized by continuous inhalation of isofluorane during treatment. The liver and portal vein was exposed by midline incision. For each mouse, ˜400 μl mixture of 50 μg plasmid and pre-activated MBs were co-injected into the liver through portal vein using a 24-G catheter for 30 sec. The liver was simultaneously treated by pulse-train US (1 sec on 2 sec off, 1.1 MHz frequency, 20 cycle pulses, 2.7 MPa PNP, 13.9 Hz PRF) for total exposure duration of 60 sec. With immediate hemostasis and suturing, the mice recovered from anesthesia within 30 min following treatment. The mice were euthanized for liver harvest on day 1 after gene transfer.

Immunofluorescent staining of GFP: 5-μm frozen cryosection of mouse liver collected on day 1 was fixed, permeabilized, and blocked with 10% serum in PBS for 30 min. The section was then stained by using anti-GFP-Alexa Fluor® conjugates (Life Technologies, Grand Island, NY, USA) at 1:200 dilution in PBS. Liver section from plasmid injected mouse without US treatment was also stained as a negative control. All sections were counterstained with UltraCruz® Mounting Medium containing DAPI (4′, 6-diamidino-2-phenylindole) (Santa Cruz Biotechnology, Inc., Dallas, TX, USA) to visualize cell nuclei. Following staining, all liver sections were examined by Leica DM6000 B fluorescence microscope system.

Luciferase expression in liver parenchymal and non-parenchymal cells: On day 1 after US/MB mediated gene transfer, hepatocytes and non-parenchymal cells were isolated from treated livers by liver perfusion and centrifuge method. Luciferase assay was performed by using Luciferase Assay System (Promega) and a luminometer (Victor 3; Perkin-Elmer, Wellesley, MA, USA). Luciferase activity was normalized to the total cell number or protein mass of either cell population, and is expressed as RLU per 10⁷ cells or RLU per mg protein.

Therapeutic FVIII gene transfer in HA/Balb/C mice with immunomodulation: For FVIII gene delivery, the mice were anesthetized and midline incisions were made as described previously. For each mouse, ˜400 μl mixture of 100 μg plasmid and 5% Vol. MBs were injected into the liver through portal vein using a 24-G catheter for 30 sec with simultaneous pulse-train US exposure (1 sec on 2 sec off, 1.1 MHz frequency, 20 cycle pulses, 2.0 MPa PNP, 13.9 Hz PRF) for 60 sec. At the end of the procedure of portal injection and US scanning, GELFOAM sterile compressed sponge (AmerisourceBergen, Chesterbrook, PA) and Bleed-X hemostatic powder (First Veterinary Supply, Livonia, MI) were applied with direct pressure to the injection site to control bleeding. On day 1 following treatment, blood samples were collected by retro-orbital bleeding for FVIII activity evaluation.

Long term study was further performed. For immunomodulation, the experimental HA/Balb/C mice was pretreated with IL-2/IL-2mAb complexes via intraperitoneal injection on day -5,-4,-3 before gene transfer of therapeutic FVIII plasmid (Liu et al., Molecular therapy: the journal of the American Society of Gene Therapy 19:1511-1520, 2011). IL-2/IL-2mAb complexes were prepared by incubating the mixture of 1 μg recombinant mouse IL-2 (PeproTech, Rocky Hill, NJ, USA) and 5 μg IL-2 mAb (JES6-1A12) (eBioscience, San Diego, CA, USA) at 37° C. for 30 min immediately before injection. In addition, to improve the bleeding diathesis of HA/Balb/C mice, 3 unit recombinant human FVIII protein and 200 μl pooled normal mouse plasma were injected (i.p.) into mouse at 30 min before surgery and 1 unit extra human FVIII protein was given at 2 hr after surgery. IL-2/IL-2mAb complexes+plasma pretreated HA/Balb/C and naïve HA/Balb/C mice were included as controls. Blood samples were collected at serial time points.

APTT and anti-FVIII assay: Human FVIII activity was measured by a modified clotting assay using FVIII-deficient plasma and activated partial thromboplastin time (APTT) reagents, and calculated from a standard curve of pooled normal human plasma (Ye et al., Molecular therapy: the journal of the American Society of Gene Therapy 10:117-126, 2004). Inhibitory antibody against FVIII was measured by Bethesda assay as previously described (Kasper et al., Thrombosis et diathesis hemorrhagica 34:612, 1975).

Tail clip bleeding assay: The mouse tail was pre-warmed in 0.9% saline at 37° C. for 2 min and subsequently cut at a 3-mm length. The cut tail was then immersed in 14 ml saline at 37° C. and blood was collected for 10 min. The bleeding was terminated by cauterizing the tail. Blood loss was quantified by the hemoglobin level of the collected blood in saline, with the absorbance measured at 560 nm.

Evaluation of FVIII encoding vector copy number in liver cells: Liver tissues were collected from treated HA/Balb/C mice on day 1 and day 4 after US mediated gene transfer. Genomic DNA was extracted using Wizard® Genomic DNA Purification Kit (Promega Corporation, Madison, WI, USA). Genomic DNA was also extracted from untreated mouse liver and hydrodynamic injection treated mouse liver as the negative and positive control. Quantitative real-time polymerase chain reaction (qPCR) of the genomic DNA was performed to measure the copy number of delivered plasmid by using Applied Biosystems Power SYBR® Green PCR Master Mix and 7500 Real-Time PCR system. The for hFVIII plasmid were primers 5′-as follows: CCAGAGTTCCAAGCCTCCAACA-3′ (forward) and 5′-GGAAGTCAGTCTGTGCTCCAATG-3′ (reverse) (Invitrogen, Carlsbad, CA, USA). The concentration of genomic DNA and the plasmid pBSHCRHP-human FVIII/N6 were measured using Spectrophotometer (NanoDrop ND-1000) and the corresponding plasmid copy number concentration was calculated using the following equation (Ye et al., Molecular therapy: J Am Soc Gene Therapy 10:117-126, 2004):

Copy number conc.(molecules/μl)=plasmid conc.(g/μl)×6.02×10²³(molecules/mol)/bp size of plasmid×330Dax2nucleotide/bp(g/mol)

A 10-fold serial dilution series of plasmid, ranging from 10¹ to 10⁸ copies/μl was used to construct the standard curve. The absolute copy numbers of FVIII encoding vector were determined by their CT values using the linear equation defined by the standard curve and normalized by extracted genomic DNA concentration

Transaminase assay: Blood samples were collected from treated or untreated control mice on day 1, 4, 7, 14, 21, 28 after gene delivery for the transaminase assay. Alanine aminotransferase (ALT) and aspartate aminotransferase (AST) levels were measured by using a commercial assay kit (Teco Diagnostics, Anaheim, CA, USA).

Histological evaluation: Treated liver tissues were harvested on day 1, 3, 7, 14, 28 after portal vein injected with plasmid/MB and US treatment. Normal mouse liver was used as control. All samples were fixed in 10% neutrally buffered formalin, embedded in paraffin, and sections were stained with hematoxylin and eosin (H&E) for evaluation of liver damage.

Statistical analysis: All data are presented as the mean±S.D. Student's T-Test was used to determine statistical significance for independent samples. Data were considered significant at P-value less than 0.05.

### Results

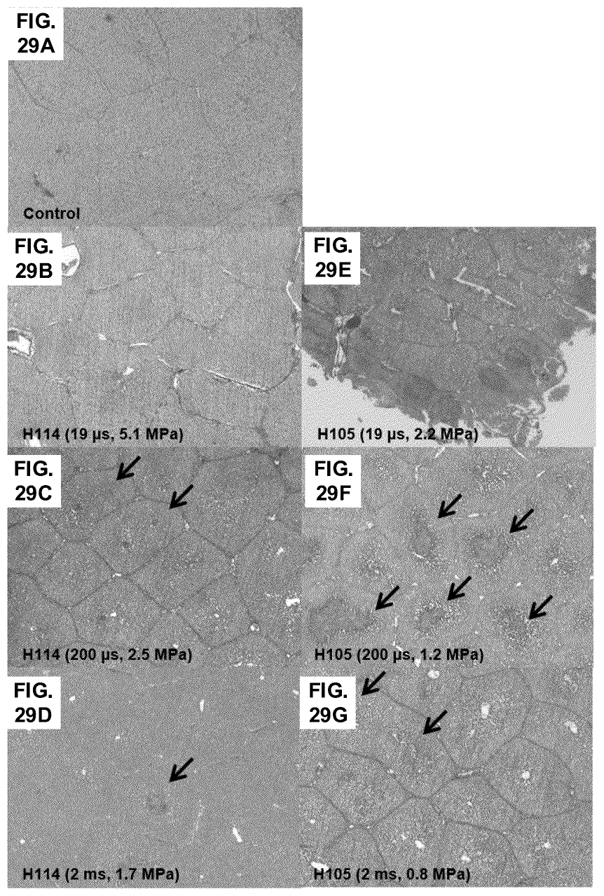

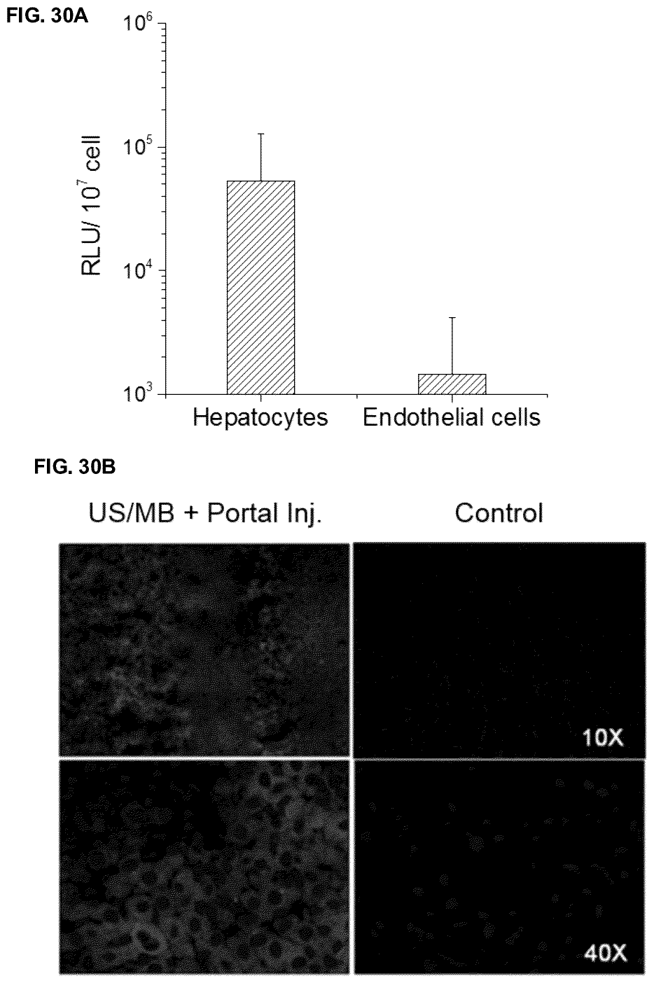

Distribution of reporter gene expression in mouse liver: In order to achieve the best therapeutic effect from delivery of FVIII plasmid, the distribution of gene delivery and expression following US/MB-mediated gene transfer of a luciferase reporter construct (pGL4.13 driven by a ubiquitous SV40 promoter) was first explored in C57BL/6 mice. A mixture of GFP plasmid and MBs was delivered through portal vain and simultaneously treated with pulse-train US at 2.7 MPa PNP for 60 sec under standard US condition (Shen et al., Gene therapy 15:1147-1155, 2008; Song et al., Gene therapy 18:1006-1014, 2011). On day 1 after gene transfer, hepatocytes and non-parenchymal cells were isolated from treated mouse livers by liver perfusion and centrifuge method and evaluated the luciferase expression from the two cell populations. The transgene expression was found in both cell types, however, the total luciferase expression levels on a per cell basis in hepatocytes (5.35×10⁴ RLU/10⁷ cells) were much higher than in non-parenchymal cells (1.46×10³ RLU/10⁷ cells; FIG. 30B). In addition, normalized by total protein level, gene expression in hepatocytes (8.32×10⁴ RLU/mg protein) is also significantly higher than that in non-parenchymal cells (1.12×10⁴ RLU/mg protein). Furthermore, a pGFP plasmid driven by a ubiquitous CMV promoter was delivered into the mouse liver by UMGD. Treated mouse liver sections were stained with fluorochrome conjugated anti-GFP to amplify the fluorescence signals. Control liver injected with plasmid only didn't show any fluorescence signal, while US/MB treated liver showed significant GFP expression (30-50% GFP+ cells). As shown in FIG. 30B, the distribution of GFP fluorescence signals was homogeneous but intensity of fluorescence appeared with a non-uniform spatial pattern. Nonetheless, within various specific local areas, the signal intensities were fairly uniform. More interestingly, GFP+signals were mostly present in hepatocytes. These data are consistent with the results obtained from the luciferase reporter construct, indicating that following US/MB delivery, transgenes were predominantly expressed in hepatocytes.

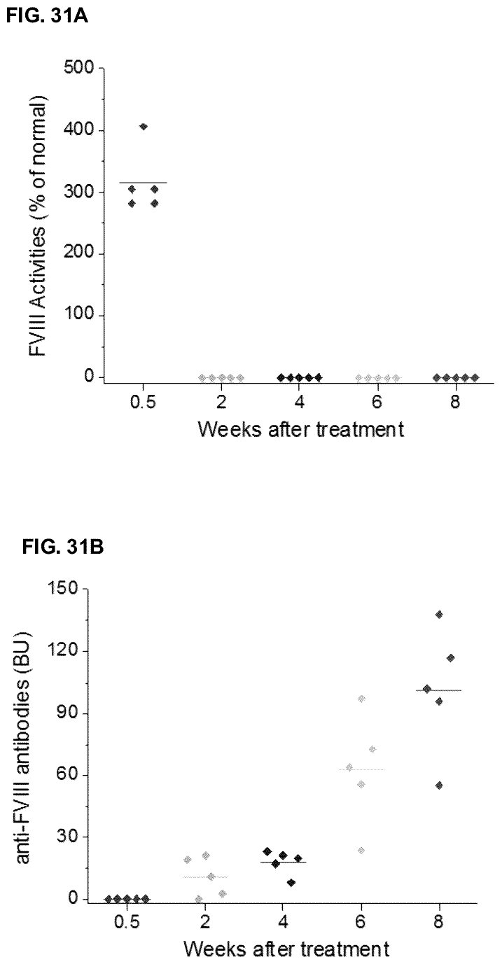

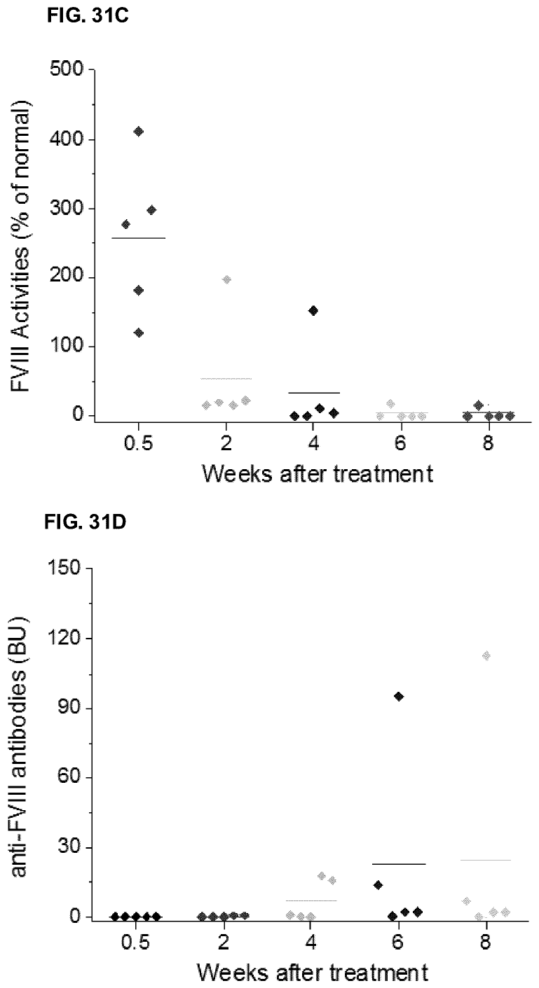

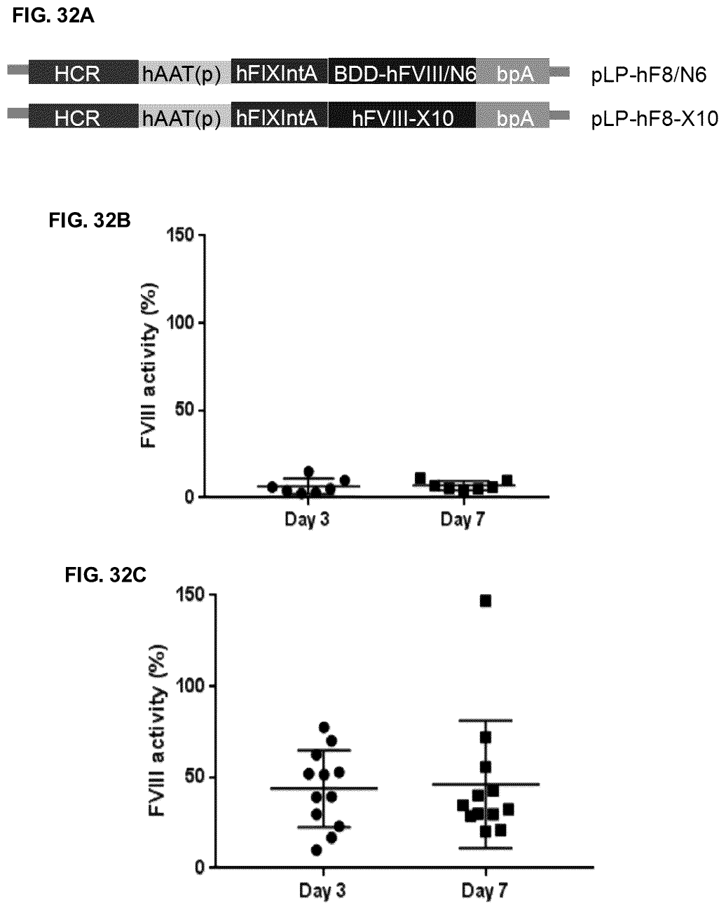

UMGD of high-expressing hFVIII variant construct achieved high-level FVIII gene expression in HemA mice. To test potential application of UMGD to treat HemA, we delivered FVIII plasmids targeting the liver. using the unfocused H158A transducer and the standard US parameters (Song et al., Mol Pharm 9:218702196, 2012) with laparotomy procedures. As demonstrated in the study of distribution of transgene expression from plasmids delivered by US/MB, hepatocyte was found to be the primary cell population of gene transfer. Therefore, we delivered a hepatocyte-specific hFVIII construct (pLP-hF8/N641; FIG. 32A) carrying a B-domain deleted (BDD)-FVIII/N6 cDNA42 driven by a potent liver-specific HCR-hAAT promoter/enhancer into the HemA mouse livers. BDD-FVIII/N6 consisted of a partial B-domain deletion leaving an N-terminal 226 amino acid stretch containing 6 intact putative asparagine-linked glycosylation which increased in vitro and in vivo secretion of FVIII by 10-15 fold and also reduced anti-FVIII immune response (FIG. 31A-31D). It should be noted that the hemophilia A mice are prone to develop anti-FVIII inhibitory antibodies within 10-14 days. However, we were able to detect FVIII expression ranging from 3-15% in mice on day 3 and 7 following UMGD treatment (FIG. 32B). Since our first target is to treat hemophilia dogs and patients who are not prone to develop anti-FVIII antibodies, we did not follow the FVIII expression for long-term in these mice. From our experience in nonviral gene transfer of FVIII plasmids in tolerized mice, FVIII expression levels usually drop several fold initially then stabilize afterwards over time. We seek to increase the levels of FVIII gene expression so that therapeutic levels can be sustained for long term.

A FVIII plasmid incorporating a modified FVIII cDNA producing FVIII protein with higher expression level, enhanced bioactivity and longer half-life would be highly desirable for successful gene therapy of HemA (Roberts et al., J Genet Syndr Gene Ther. 1, 2011). Compared with human FVIII, canine FVIII(Wang et al., Gene Therapy 23:597-605, 2016) is functionally more active, whereas porcine FVIII (Doering et al., J Biol Chem 277:38345-38349, 2002) expresses at higher levels. Recently, a variant hFVIII cDNA with 10 amino acid porcine FVIII-like substitutions in the A1 domain of the FVIII heavy chain, hF8-X10 (Cao et al., Am Soc Gene Cell therapy, 17th Annual Meeting, Abstract 460, 2014), significantly improved FVIII gene expression levels. We have incorporated this FVIII variant into our liver-specific plasmid vector to generate pLP-hF8-X10 (FIG. 32A). When we delivered this plasmid into the HemA mice by hydrodynamic delivery, more than 10-fold higher FVIII gene expression was obtained compared with the control construct pLP-hF8/N6A. When we used UMGD to transfer pLP-hF8-X10 containing the FVIII variant into HemA mice (n=12), we obtained 30-77% FVIII gene expression on day 3 and 30-150% on day 7 (FIG. 32C). These data indicated that high-level FVIII gene expression could be achieved following UMGD in HemA mice.

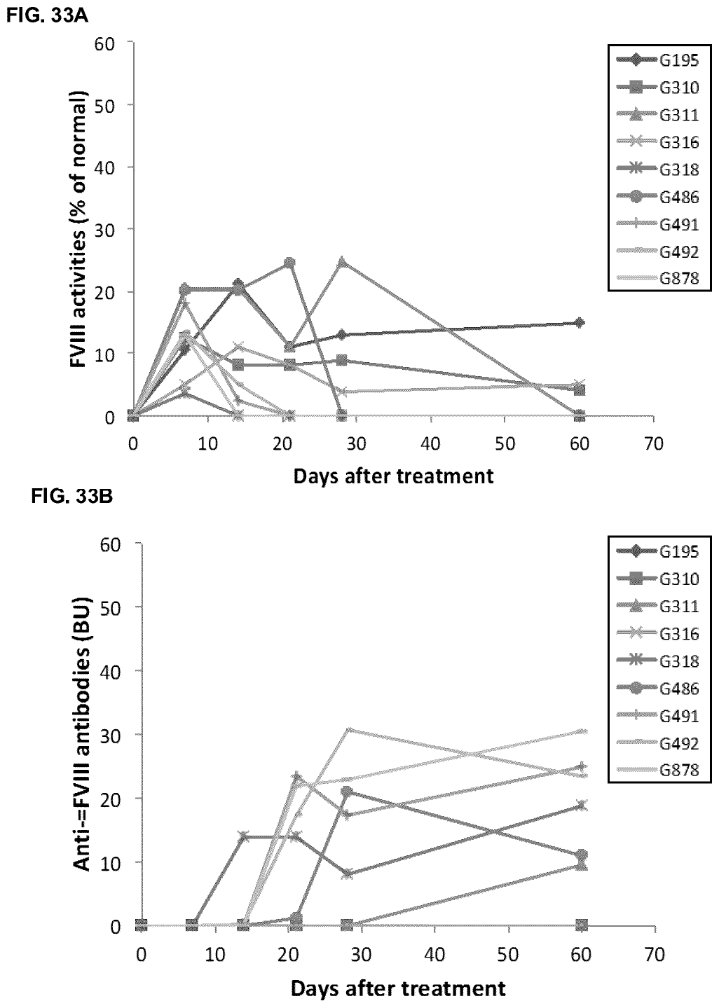

In longer term follow up of gene expression, both control mice and US/MB treated mice injected with FVIII plasmid containing the BDD-FVIII cDNA, FVIII became undetectable 2 weeks post injection and anti-FVIII antibodies were generated. In order to modulate anti-FVIII immune responses, experimental mice were pretreated with IL-2/IL-2 mAb complexes to prevent the formation of high-titer inhibitory antibody responses (Liu et al., Molecular therapy: the journal of the American Society of Gene Therapy 19:1511-1520, 2011). Normal mouse plasma 200 μl and human FVIII protein 3 units were given to the mice to maintain hemostasis prior to surgery. As shown in FIG. 33A, 10-20% FVIII activity was achieved by day 7. However, due to pretreatment of FVIII protein and gene transfer, most of the treated mice still produced low-titer inhibitors starting from day 14 (FIG. 33B). On day 28, FVIII activities persisted in the average level of 10% at low antibody levels. Three of the mice still maintained 5-15% activity after 60 days (FIG. 33A). Interestingly, FVIII levels inversely correlated with the titers of corresponding anti-FVIII antibodies at various time points. Taken together, these data indicated that long-term and therapeutic FVIII gene expression can be achieved following UMGD in a hemophilia mouse with successful immunomodulation.

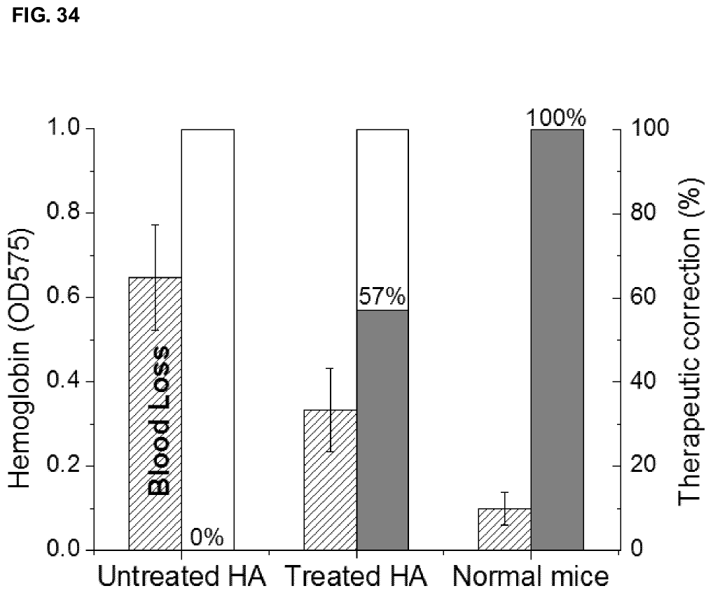

Correction of the bleeding disorder in hemophilia A mice: In order to evaluate the phenotype correction of hemophilia A mice after US/MB mediated gene transfer of hFVIII plasmid, a tail clip bleeding assay was performed on treated and untreated HA/Balb/C mice, as well as normal mice. Blood loss during 10 min time period following mouse tail transection was evaluated using hemoglobin levels. In FIG. 34, significantly larger amount of blood loss was observed in untreated hemophilia A mice compared to wild-type normal mice. Total blood loss was significantly reduced in treated mice compared with untreated hemophilia A mice, although still higher than normal mice. Most of hemophilia A mice treated with US/MB mediated gen transfer showed partial phenotype correction of hemophilia A with some variability for individual mouse. In average, 58% therapeutic correction was achieved compared with normal mice as positive control (100%) and untreated HA/Balb/C as negative control (0%).

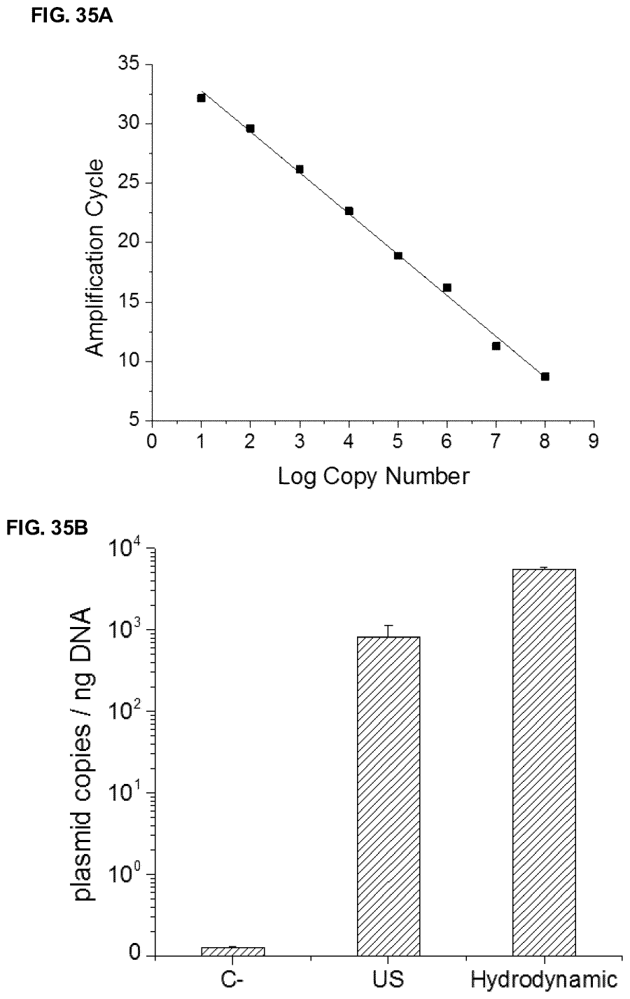

Evaluation of plasmid copy number in mouse liver: A standard curve from serial 10-fold dilutions of hFVIII plasmid was generated by SYBR-based qPCR, as shown in FIG. 35A. The slope of standard curve equals-3.45, with coefficient of 0.996, indicating a high PCR efficiency of 95%. In addition, the melting curve analysis showed only one peak, confirming the specificity of amplicon. Absolute quantification of FVIII encoding vector copy number in genomic DNA was determined by relating CT value to the standard curve. The copy numbers were then normalized by DNA quantities for different HA/Balb/C groups (FIG. 35B), including untreated mice as negative control, the mice treated by hydrodynamic injection as positive control, and US/MB treated mice. US treated mice showed significantly higher copy numbers of vector DNA on days 1 and 4 compared with untreated mice. However, compared to mice treated with hydrodynamic injection, the vector copies in the liver are still ˜8 fold lower.

Mouse liver damage: Transaminase levels (ALT and AST) were examined to assess liver functional damages associated with US/MB mediated gene transfer (Table 4). Compared to untreated control mice, treated mice had significantly increased ALT levels (˜1035 IU/L) and AST levels (˜524 IU/L) on day 1 after treatment. However, both ALT and AST dramatically decreased on day 4 (ALT ˜37IU/L; AST ˜87IU/L) and quickly recovered to control levels by day 28. The liver enzyme levels indicated that the procedure of plasmid/MBs portal-vein injection and pulse-train acoustic exposure produced transient liver functional damages however the damages were repaired rapidly.

TABLE 4
Alanine transaminase (ALT) and aspartate transaminase (AST)
of plasma collected from hemophilia A mice treated with portal
vein injection and simultaneous 2.0 MPa pulse-train US.

| | Days after treatment | | | | | |
|---|---|---|---|---|---|---|
| | 1 | 4 | 7 | 14 | 28 | C− |
| ALT | 1035 ± 254 | 37 ± 17 | 19 ± 7 | 44 ± 13 | 29 ± 2 | 21 ± 6 |
| (IU/L) | | | | | | |
| AST | 524 ± 297 | 87 ± 13 | 61 ± 10 | 96 ± 8 | 79 ± 10 | 77 ± 15 |
| (IU/L) | | | | | | |

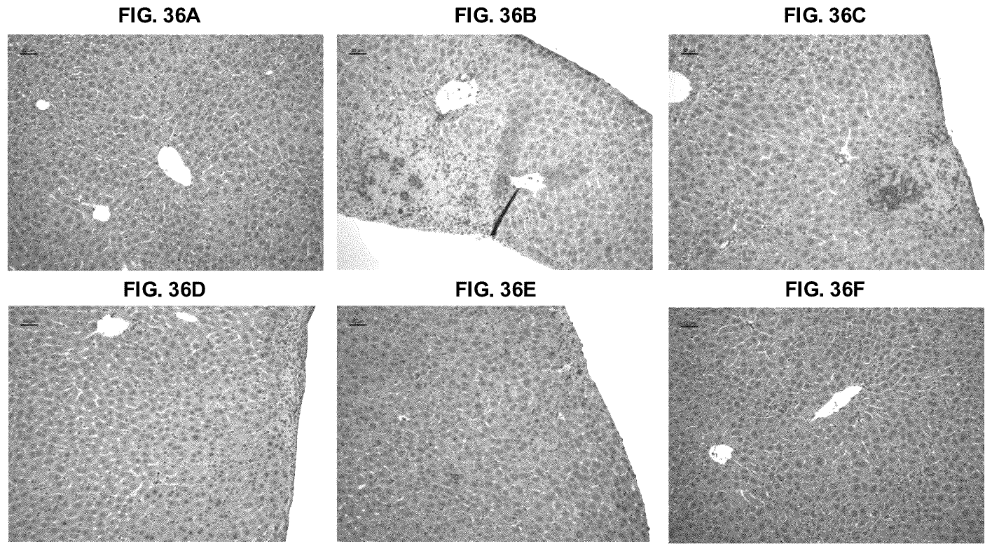

Liver damages were further determined by H&E staining of tissue sections, which were collected from portal injection+US treated HA/Balb/C mice on day 1, 3, 7, 14 and 28 after treatment (FIG. 36B-36F). Untreated mouse liver was also examined as control (FIG. 36A). The representative sections in the figure were intentionally selected to present the types of hepatic damages and the areas with maximal extent of injury. On day 1 after treatment, there was some focal coagulative necrosis and hemorrhage mostly close to treated liver surface. The extent of necrosis was variable in sections from different treated liver lobes, ranging from 10˜30% of total area of the section examined. There was sharp demarcation between the areas of necrosis and viable tissue, with scattered apoptotic hepatocytes near the boundary. Some extracellular micro-vesicles and dilatation of sinusoid were observed, probably due to plasmid/MBs fluid injected through portal vein. The area of necrosis with scattered hemorrhage and extravasated erythrocytes considerably decreased on day 3 post-treatment, and lymphocytes infiltration appeared. On day 7, liver damages were remarkably diminished and repaired, only with a small amount of scattered hemorrhage. In a few small areas of treated surface, granulation and fibrous tissue was present, which was probably caused by manipulation of US scanning and application of US coupling gel. Damages were mostly repaired and recovered to normal by day 14 after treatment.

Discussion: US combined with MB has been applied for gene transfer as a non-viral physical delivery strategy. An unfocused US system with MBs has previously been developed and optimized to significantly enhance gene transfer efficiency of reporter plasmids in murine models (Song et al., Molecular pharmaceutics 9:2187-2196, 2012; Shen et al., Gene therapy 15:1147-1155, 2008; Song et al., Gene therapy 18:1006-1014, 2011). In this study, an efficient non-viral gene delivery method for hemophilia A gene therapy was developed by applying US/MBs to deliver FVIII naked DNA into the mouse liver.

First, the distribution of reporter gene expression from plasmids delivered by US/MBs was investigated. The luciferase gene expression was examined in parenchyma (hepatocytes) and non-parenchyma cells including sinusoidal endothelial, Kupffer, stellate, and othercells. These results demonstrated that plasmids were delivered and expressed in both parenchyma and non-parenchyma cells. However, the transgene was predominantly expressed in hepatocytes by US/MBs at current conditions. The distribution of transgene was further explored using a GFP reporter plasmid. The fluorescent staining of treated liver sections showed that 30-50% of liver cells had GFP expression. Compared to US/MBs mediated gene transfer with intrahepatic administration of plasmid/MBs mixtures in a previous study (Miao et al., Human gene therapy 16:893-905, 2005; Shen et al., Gene therapy 15:1147-1155, 2008), there was a greater percentage of cells expressing GFP (30-50% vs. 5-10%). This might be due to the following improvement of treatment procedure: (1) the delivery route of portal vein injection allowed the fluid of plasmid and MBs to spread more evenly in the liver tissue through capillary vessels; (2) the treatment regimen of pulse-train US provided longer quiescent periods between US pulses to allow reperfusion of plasmid and MBs, achieving more sustained distribution in the live tissue; (3) the unfocused US transducer with larger-footprint could treat larger tissue volume with more homogeneous average intensity of US exposure. In addition, GFP+signals are predominantly present in hepatocytes and homogeneously distributed in spatially localized areas. This is different from hydrodynamic based gene transfection (Miao et al., Human gene therapy 16:893-905, 2005; Miao et al., Human gene therapy 14:1297-1305, 2003; Ye et al., Journal of thrombosis and Haemostasis: JTH 1:103-111, 2003, which showed higher intensity but in more concentrated and scattered areas. However, the intensity of GFP expression exhibited regional variability in the whole live section, suggesting that in the presence of MBs, US induced inertial cavitation was probably in some extent restricted by liver tissue size, physiological structure, and capillary vessel distribution, etc., These factors would influence the flow distribution of MBs in liver tissue, propagation, scattering and attenuation of US in tissue, vessel wall rupture and permeability, leading to regional difference in expression intensity. In any case, the predominance of gene expression in hepatic cells is consistent with what was observed in luciferase gene transfer study.

Based on this finding of gene expression distribution, a human FVIII plasmid driven by hepatocytes-specific promoter was employed in therapeutic gene transfer for hemophilia A mice. In a previous study, it was found that gene expression was correlated with liver damage induced by acoustic cavitation of US/MBs. In previous normal mouse experiments, gene expression increased as acoustic peak negative pressure (PNP) increased at the range of 0-3.2 MPa, and presented a gradual plateau trend at PNP >2 MPa while liver damage increased more rapidly. Although gene transfer efficiency will be enhanced by higher acoustic pressure, it is also critical to minimize any potential liver damage. Therefore, in this study 2 MPa of PNP was used for treatment of hemophilia mice, instead of higher pressure, in order to obtain desired gene expression with minimal tissue damage. In addition, the pulse-train US procedure (1 sec on 2 sec off) was employed to allow more efficient perfusion of plasmid/MBs mixtures in the liver and further decrease the liver damages. Previously it has been shown that pulse-train US procedure using ⅓ of the US energy could produce comparable or even higher gene transfer efficiency compared with continuous US pulses, therefore significantly decreasing acoustic cavitation induced tissue damages (Song et al., Molecular pharmaceutics 9:2187-2196, 2012).

Using the pulse-train US procedure at modest acoustic pressure, treated hemophilia A mice showed a significant enhancement of hFVIII activity on day 1 post-treatment compared to untreated mice. For longer term follow up, a separate group of mice was treated with hFVIII protein as well as normal mouse plasma to control bleeding during and after surgery. The experimental mice were also treated with immunomodulation therapy with IL2/IL2mAb complexes according to the successful protocol in a previous study to prevent the FVIII-specific immune responses following gene transfer (Liu et al., Molecular therapy: the journal of the American Society of Gene Therapy 19:1511-1520, 2011). To avoid the influence from pre-injected FVIII protein and plasma on FVIII activity, data was collected from day 4. It showed that therapeutic levels were achieved initially, and then FVIII activity presented a fluctuation trend associated with the opposite tendency of anti-FVIII levels. Within 28 day after gene transfer FVIII activities mostly persisted in the average level of 20%, however they declined on day 60 and presented a larger individual variation afterwards (0-45%). On the contrary, inhibitory antibodies occurred on day 14 after treatment and appeared a low level until day 28, following with a significant increase on day 60 and varying greatly between 60 to 120 days. Based on initial significant enhancement of FVIII expression, as well as correlation of FVIII gene expression levels and inhibitory FVIII antibodies, it indicated continuous FVIII gene expression mediated by US/MBs, although long-term gene expression was inhibited by transgene-specific immune response against hFVIII. IL2/IL2 mAb complexes were reported to effectively inhibit inhibitory antibody formation following FVIII gene therapy. In this study, similar immunosuppressive treatment of IL2 complexes were followed, however the immune responses was only partially modulated following gene transfer mediated by US. This might due to multiple possible factors, such as immunogenicity of different mouse strains, infusion of recombinant human FVIII protein, plasmid and animal health condition after surgery (Qadura et al., Haemophilia: the official journal of the World Federation of Hemophilia 17:288-295, 2011). It suggests that a more effective immunomodulation strategy is required for persistent long-term tolerance to FVIII gene expression produced by US/MB.

The difference of plasmid copy levels between two groups is less than the difference of luciferase gene expression (US treated group is 50-100 fold lower than hydrodynamic injection treated group), which might be due to the limited plasmid nuclei (Song et al., Gene therapy 18:1006-1014, 2011).

For gene therapy of hemophilia A mice, liver damage has a more important impact on gene transfer efficiency than that in normal mice, which could lead to serious bleeding, hepatic apoptosis or even possible mortality of HA mice. As mentioned above, pulse-train US exposure at 2 MPa was employed for treatment to minimize liver damage induced by acoustic cavitation. Transaminase levels presented a rapid increase on day 1 but decreased very soon back to normal level within 7 days after treatment, indicating a similar pattern of acute liver functional damage compared to that in previous normal mice study (Song et al., Molecular pharmaceutics 9:2187-2196, 2012; Miao et al., Human gene therapy 16:893-905, 2005). Consistently, in histological examination, hepatic damage type and the pattern of tissue repair were also similar to what was found in normal mouse and rat studies, appearing coagulative necrosis with hemorrhage and inflammation in the process of injuring and repairing. However, it was noticed that, in treated liver of hemophilia mice, there was still a small amount of hemorrhage until day 7 after treatment, which is rare to appear in treated normal mice or rats. Initial mild bleeding from the mechanical tissue damage caused by injection/US was extended and prolonged in Hemophilia mice during tissue repairing due to their FVIII deficiency. In spite of this marginal histological difference between hemophilia A mice and normal mice or rats, treated livers of hemophilia A mice similarly revealed rapid and significant recovery and repaired completely within 14-28 days post-treatment. In all sections, same as previously shown (Song et al., Molecular pharmaceutics 9:2187-2196, 2012; Miao et al., Human gene therapy 16:893-905, 2005), there was no thermal denaturation observed, indicating no thermal effect at current US condition. Looking at the above data of transaminase levels and histology, despite showing slightly more hemorrhage, hemophilia mice presented a pattern of liver damages similar to normal mice, which was considered to be induced by an inertial cavitation-mediated mechanism.

Taken together, on the basis of previous reporter gene transfer studies, the feasibility of applying US/MBs delivery system in therapeutic plasmid gene transfer was further explored. Mouse liver was surgically exposed and injected with plasmid/MBs through portal vein and treated with US scanning. This treatment was a direct and applicable procedure of simultaneous MBs infusion and US exposure for mouse model. For further larger animal models and clinical application, minimally invasive procedure is being developed by infusing plasmid/MBs into liver with interventional radiology technique and applying US transcutaneously. This report constitutes a proof of principle study and demonstrates successful transfer of FVIII plasmid into hemophilia A mouse model by US/MBs. Significant FVIII expression levels and partial phenotypic correction of hemophilia A were achieved although long-term therapeutic FVIII levels were undermined by the formation of FVIII-specific inhibitory antibodies. This study provided a promising strategy for efficient and safe treatment of hemophilia A, which is the first step of US/MBs mediated gene therapy towards hemophilia clinical application.

### Example 5: Multivariate Ultrasound Signal Manipulation for Effective Gene Delivery to Pig Livers

Previously, ultrasound (US) transducer piezomaterial limitations were surmounted in US-mediated gene delivery (UMGD) while achieving significant enhancement of nonviral gene transfer in mice. This was accomplished by prolonging pulse duration (PD) and lowering peak negative pressure (PNP). The goal was to scale-up the results in a porcine model, and to further develop transducers planned for minimally invasive UMGD. pGL4 (encoding a luciferase reporter gene) plasmid DNA and microbubbles were infused into a portal vein branch of a porcine liver lobe and exposed to US for four minutes. Over 100-fold enhancement in luciferase expression was obtained in pig livers compared to sham-treated using a cylindrically focused, three-element transducer (H185D, center frequency (fc)=1.05 MHZ) and a (19 μs, 6.9 MPa) US protocol. Over 50-fold enhancement in gene expression was obtained relative to sham when performing UMGD transcutaneously. Transcutaneous expression was further enhanced when the transducer focal depth was revised to 30 mm (H185F, fc=1.07 MHZ). Equivalent enhancement of transgene expression was attained when lowering PNP and lengthening PD (200 μs, 4.6 MPa; 2 ms, 2.6 MPa) for both transcutaneous and open UMGD procedures. These results demonstrate the advancement of safe UMGD technology and its potential for clinical application.

### INTRODUCTION

Ultrasound-mediated gene delivery (UMGD) has been demonstrated to be a viable method for gene transfer. Key advantages of UMGD over viral methods include low toxicity and reduced immunogenicity (Miao & Brayman, Ultrasound-mediated gene delivery, Non-viral gene therapy (Yuan X, Ed.), Intech, Rijeka, Croatia (2011) pp213-242; Noble-Vranish et al., Mol Ther Methods Clin Dev, 10:179-188, 2018; Liu et al., Oncol Rep, 34:2977-2986, 2015; Mead et al., J Control Release, 223:109-117, 2016). Additionally, construct size in UMGD is much less constrained, accommodating larger size genes. Finally, it can target gene delivery to specific tissue/cells (Mead et al., J Control Release, 223:109-117, 2016; Chang, et al., J Control Release, 255;164-175, 2017; Fan et al., Biomaterials, 106:46-57, 2016; Noble et al., Mol Ther, 21:1687-1694, 2013; Song et al., Gene Ther, 18:1006-1014, 2011). Gene transfer efficiency in UMGD is enhanced by the presence of exogenous nuclei such as lipid-shelled microbubbles (MBs). The MBs may oscillate radially at relevant driving frequencies and applied pressures, termed cavitation. These cavitation nuclei may oscillate violently and collapse under certain ultrasound (US) parameter settings. Collapse of the cavitation nuclei can lead to transient permeabilization of the vascular endothelium and pore formation along the cell membrane of target cells-called sonoporation-allowing entry of plasmid DNA (pDNA). This use of UMGD in combination with nonviral gene therapy is motivated by the primary aim of treating genetic diseases safely with comparable gene transfer efficiency to viral methods.

The liver is a desirable target for gene therapy as it is a primary site of important metabolic pathways and serum protein production. It has previously been demonstrated the ability to perform efficient gene transfer to the liver using UMGD in both small and large animals (Noble-Vranish et al., Mol Ther Methods Clin Dev, 10:179-188, 2018; Noble et al., Mol Ther, 21:1687-1694, 2013; Song et al., Gene Ther, 18:1006-1014, 2011; Song et al., Mol Pharm, 9:2187-2196, 2012). In a recent swine study, further improvement of gene transfer efficiency was attempted to exploring different transducer designs (Noble-Vranish et al., Mol Ther Methods Clin Dev, 10:179-188, 2018). Using a cylindrically focused transducer, H185D, it was possible to achieve up to 9000-fold increase in average transgene expression relative to sham. Using H185D with a 19 μs pulse duration required a peak negative pressure (PNP) at the focus of at least 2.7 MPa for effective transfection in swine. Transfection enhancement was further increased up to PNPs of 6.9 MPa, the max rated pressure limit for that transducer.

These advances in selection of appropriate US transducers and conditions have moved us towards the secondary goal of developing a minimally invasive, clinically relevant procedure to perform UMGD. A component of that goal is performing UMGD transcutaneously, thereby circumventing the need for a laparotomy and reducing associated surgical risks. This is a substantial transition, and one associated challenge is the attenuation of acoustic power that occurs across several intervening tissue layers. For a given transducer, this attenuation reduces the maximum pressure attainable in the target liver tissue. While higher pressures could be achieved by using a more tightly (e.g. spherically) focused transducer design, prior studies have shown that the resulting smaller treatment volume produces decreased expression when compared to cylindrically focused designs of the same diameter (Noble-Vranish et al., Mol Ther Methods Clin Dev, 10:179-188, 2018). To a lesser extent, higher pressure could also be achieved by using a larger transducer, but experience indicates that transducers such as H185D are near the upper limit of size that can be manipulated to target the liver in transcutaneous treatment without risking off-target treatment of either the ribcage or intestines. Because of these constraints, other US protocols were investigated, varying parameter settings such as PNP and pulse duration (PD), that would allow effective gene transfer at a lower applied acoustic pressure. It was found that prolonging the PD of an US cycle lowers the acoustic pressure threshold for efficient transfection in HEK293T cells and mice (Tran et al., J Control Release, 279:345-354, 2018). Here, the feasibility of scaling recent findings to a large animal model was examined. Early attempts to perform UMGD transcutaneously with the intention of facilitating translation of safe US and surgical protocols to clinical application are also presented.

### Materials and Methods

Plasmid and MB preparation: A luciferase reporter plasmid with an SV40 promoter, pGL4.13 [luc2/SV40] (Promega, Madison, WI) was produced by GenScript Inc. (Piscataway, NJ) according to standard industry techniques. Preparation and characterization of RN18 MBs was described previously by Sun et al (Sun et al., J Control Release, 182:111-120, 2014). Briefly, the MB shells were comprised of lipids at an 82:10:8 molar ratio of 1, 2-distearoyl-sn-glycero-3-phosphocholine (DSPC), 1,2-distearoyl-sn-glycero-3-phosphate (DSPA), and N-(Carbonylmethoxypolyethyleneglycol 5000)-1,2-distearoyl-sn-glycero-e-phosphoethanolamine (MPEG-5000-DSPE) (Avanti@ Polar Lipids, Alabaster, AL). The lipids were reconstituted in PBS, 10% glycerol, and 10% ethylene glycol (Sigma-Aldrich, St. Louis, MO) in a 3-mL vial and sealed. Gas-exchange was performed, and the headspace was filled with octafluoropropane gas (American Gas Group, Toledo, OH). Before use, MBs were produced by vigorous agitation of the lipid emulsion for 45 s using a Vialmix™ (Lantheus Medical Imaging, N. Billerica, MA), yielding an average concentration of 2-5×10⁹ MBs/mL and an average size of 1.5 μm.

Transducer and US systems: A laptop was used to control a signal-generating amplifier (Model: RFG-1500BB, JJ&A Instruments, Duvall, WA; Model: RPR-4000-HP, Ritec Inc, Warwick, RI) via custom serial interface (Sonic Concepts, Bothell, WA). The combined pulse generator and radio-frequency power amplifier was connected to an impedance matching network to minimize reflections and maximize power transfer. The matching network was subsequently connected to a three-element, 49 mm diameter, cylindrically focused transducer (Model H185D, Sonic Concepts). The lens focus is 20 mm from the exit plane of the transducer with a linear focal pressure gain of 4.46. US application was driven at a center frequency of 1.05 MHz. US treatment monitoring was performed by capturing current, voltage, and calculated power from a gated, triggered waveform using a high sample-rate oscilloscope (44MXs-B, Teledyne Lecroy, Chestnut Ridge, NY).

A second three-element, 49 mm diameter, cylindrically focused transducer (Model H185F, Sonic Concepts) was used for semi-open UMGD. The lens focus is 30 mm from the exit plane of the transducer with a focal pressure gain of 2.73. US application was driven at a center frequency of 1.07 MHz. Effective treatment areas for H185D and H185F are approximately 1.6 cm², defined as the total-6 dB enclosed area across all three focuses in the XY (parallel to the face) plane at the transducer focal depth.

Each transducer is coated with a thin film of epoxy and the housing is plastic. The transducer face, housing, and cable exit are watertight. The transducer interior was filled with acoustic backing material to make them hermetic throughout. A 1.0 mm diameter hydrophone was used to measure absolute pressure of the transducers at the focus using a 1 MHZ fundamental frequency and normalized to 1 MPa peak pressure at acoustic maximum.

Ultrasound Propagation Simulations: All simulations were performed within MATLAB (Mathworks, Natick, MA) using the k-Wave toolbox v2.1, which employs a k-space pseudo-spectral time domain solution (Treeby et al., J Acoust Soc Am, 131:4324-4336, 2012). The computational grid was 512×512 points including a perfectly matched layer (PML) on all sides that was 20 points in width. Solutions were found using a first-order solution, accounting for attenuation and non-linear effects. The pressure signal at the transducer surface was modeled as a dirichlet source following a damped sinusoid with maximum amplitude determined based on physical measurements of each transducer made by the manufacturer. Composition and thickness of layers for transcutaneous simulation were determined by dissection of an intervening tissue sample that was collected from a pig post-mortem. Constituent layers were separated, and their thicknesses were measured using a dissection probe needle and Vernier calipers. These measurements were corroborated by comparing against distance measurements performed using diagnostic ultrasound prior to any surgical incisions.

Furthermore, in order to assess potential thermal damage from at least a first-order estimate, the k-Wave model was extended to calculate anticipated post-treatment temperatures using a 2D implementation of the Pennes' Bioheat Equation. This thermal model showed that the maximum power used at all pulse durations for each transducer produced a CEM43 thermal dose of 0 across the transducer field. This result indicates no anticipated thermal damage produced as a result of the treatment conditions. Typical acoustic and thermal constants were used for all the simulations (Azhari, Basics of biomedical ultrasound for engineers, Wiley: IEEE, Hoboken, N.J., 2010; Hasgall et al., IT′IS Database for thermal and electromagnetic parameters of biological tissues, Version 4.0 (2018) DOI: 10.13099/VIP21000-13004-13090. itis.swiss/database).

Porcine surgery: All procedures were performed according to the guidelines for animal care of both the National Institutes of Health and Seattle Children's Research Institute, with protocol approval of Institutional Animal Care and Use Committees. S.P.F. derived Yorkshire hybrid swine (8-15 kg, Male or female) were obtained from S&S Farms (Ramona, CA). The swine were acclimated to their new housing in the SCRI vivarium for at least four days prior to surgery. The animal numbers used in each set of experiments were determined in reference to Eng (Radiology 227 (2): 309-313, 2003). Simple randomization was used to allocate animals to different groups.

After induction of general anesthesia (ketamine/xylazine subcutaneously, and 3% isofluorane by inhalation), each animal was placed in the supine position. Surgeries were performed as previously described by Noble et al. (Noble-Vranish et al., Mol Ther Methods Clin Dev, 10:179-188, 2018). Briefly, the abdomen was shaved, prepped and draped in a sterile fashion. A midline incision was made, and a Balfour retractor was used to further expose the liver. An angiocath was inserted in the portal vein (PV) segmental branch leading to the left lateral lobe and subsequently connected with an extension set to a syringe containing the pDNA/MB solution. The inferior vena cava (IVC) was occluded prior to injection of pGL4/MB solution (2 mL/kg solution containing: 0.67 mg/kg pGL4, 0.2 mL/kg MBs, 0.2 mL/kg 50% glucose, and PBS to total volume) to retain MBs within the liver. Shortly after occlusion and simultaneous to injection, US was applied on the liver surface for four minutes (1.05 MHz center frequency, 50 Hz pulse repetition frequency). Sham pig experiments received the same pDNA/MB treatment and an identical application of a transducer which was disconnected from any power source. For semi-open UMGD, the peritoneal cavity was filled with 37° C. normal saline to ensure acoustic coupling, and the midline incision was temporarily closed and held together by forceps to allow the skin to cover the surface of the liver. US was subsequently applied on the skin directly over the position of the target liver lobe (1.07 MHz, 50 Hz pulse repetition frequency). The pGL4/MB solution was injected at a rate of 3 mL/10 s.

The MB distribution in the target liver lobe was visualized using a 4V1 vector array transducer connected to an Acuson Sequoia C512 imaging system (Siemens, Mountain View, CA) before and after US treatment. Afterwards, cannulation sites were repaired, and the incision was closed using sutures and surgical staples. Post-operative local (lidocaine) and systemic (ketoprofen) analgesics were administered during recovery. After 24 hours, the pigs were sacrificed. The treated and control lobes were sectioned and processed for luciferase expression. For sham experiments the (sham) treated and control lobes were harvested identically, and tissue from the sham-treated lobe was used to obtain results for the sham group.

Gene expression evaluation by luciferase assay: Harvested lobes (right and left lateral lobes) were resected immediately following euthanasia, wrapped in foil, and covered with dry ice. The right lateral lobe, which was not directly injected with the pGL4/MB solution and was not exposed to US, was collected as control for comparison with the treated lobe (left lateral lobe). The treated and control lobes were then sectioned into smaller pieces (˜2-3 g) and spatially mapped based on Cartesian coordinates, which were then assayed for luciferase expression as previously described (Noble-Vranish et al., Mol Ther Methods Clin Dev, 10:179-188, 2018; Noble et al., Mol Ther, 21:1687-1694, 2013). Briefly, tissues were homogenized in reporter lysis buffer (Promega, Madison, WI), and underwent three freeze-thaw cycles wherein the supernatant was collected after centrifugation. Luciferase activity was induced using a commercially available kit (Luciferase Assay System, Promega) and measured by a luminometer (Victor 3; Perkin Elmer, Wellesley, MA). Luciferase expression was normalized to total protein content, measured by BCA assay kit (Bio-Rad, Hercules, CA), and reported as relative light units (RLU)/mg protein. These assays were performed as blinded tests.

Blood analysis: Blood samples were collected before euthanasia and sent to a commercial veterinary diagnostic laboratory (Phoenix Central Laboratory, Mukilteo, WA) for a complete blood count and chemistry panel including alanine- and aspartate-aminotransferase (ALT and AST, respectively) to determine liver damage. These assays were also performed as blinded tests.

Histological analysis: Treated and control liver biopsies were fixed in 10% neutral buffered formalin, then processed and embedded in paraffin. Routine hematoxylin and eosin and trichrome-stained slides were made to determine liver damage and evaluated by a clinical pathologist (K.R.L.). These analyses were carried out as blinded tests.

Statistical analysis: All data are presented as means±SD. All statistical analyses were performed using a one-way Analysis of Variance (ANOVA) with post-hoc analysis correcting for multiple comparisons using a Tukey test (GraphPad Prism7, San Diego, CA). P-values of less than 0.05 were considered to be statistically significant.

### Results

Prolonged pulse duration can lower acoustic pressure threshold for gene delivery in porcine livers: In a previous cell and mouse study, the effects of prolonging PD on the acoustic pressure threshold needed for MB cavitation and subsequent gene transfer were investigated. It was found that the pressure threshold for gene delivery can be lowered by prolonging the PD while also enhancing gene transfer (Tran et al., J Control Release, 279:345-354, 2018). Several different US transducers were developed with Sonic Concepts. It was found that the cylindrically focused H185D transducer significantly enhanced gene transfer relative to other transducer variants and was most effective when using a PNP of 6.9 MPa (Noble-Vranish et al., Mol Ther Methods Clin Dev, 10:179-188, 2018). Hence, this specific transducer was used to perform swine studies. The experimental conditions evaluated for UMGD in swine are summarized in (Table 5). The H185D transducer was used to deliver PNPs varying between 0 MPa (sham; transducer applied, but not activated) and 6.9 MPa for a total exposure time of 4 minutes. The pGL4/MB solution was injected into a portal vein branch, with the IVC occluded to retain MBs within the liver, and the transducers were scanned at a rate of 1 cm/s across the targeted liver surface. The total volume of pGL4/MB solution injected also varied depending on the pig weight from 19 mL to 31 mL containing 1.9-3.1 mL of RN18 MBs in solution. The pDNA/MB solution was injected simultaneously to US treatment at a rate of 3 mL/10 s and then flushed with 3 mL of PBS to ensure full injection of pDNA/MBs. After 24 hours when luciferase expression had peaked, the swine were sacrificed, and the treated lobe (left lateral lobe) and control lobe (right lateral lobe) were harvested and assayed for luciferase expression.

TABLE 5
Summary of experimental conditions used in ultrasound-mediated gene delivery

| | | | Focal | | | | | |
|---|---|---|---|---|---|---|---|---|
| | | Frequency | Depth | PNP | Pulse | | Duty | Interpulse |
| Group | Transducer | (MHz) | (mm) | (MPa) | Duration | | Cycle | Interval |
| Sham | — | — | — | 0 | — | | — | — |
| 1 | H185D | 1.05 | 20 | 6.9 | 19 | μs | 0.095% | 20 ms |
| 2 | H185D | 1.05 | 20 | 4.6 | 200 | μs | 1% | 20 ms |
| 3 | H185D | 1.05 | 20 | 2.6 | 2 | ms | 10% | 18 ms |
| 4 | H185F | 1.20 | 30 | 6.9 | 19 | μs | 0.095% | 20 ms |
| 5 | H185F | 1.20 | 30 | 2.6 | 2 | ms | 10% | 18 ms |

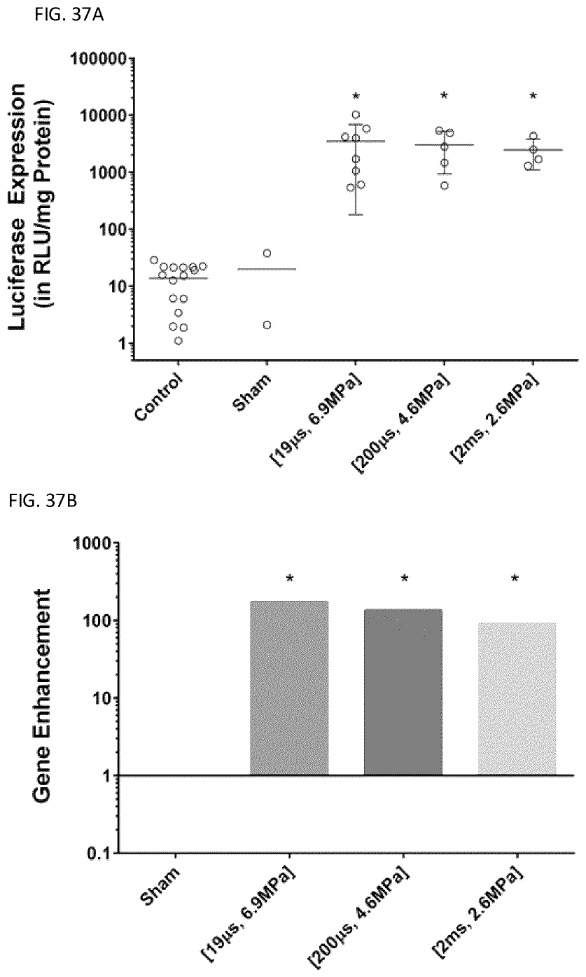

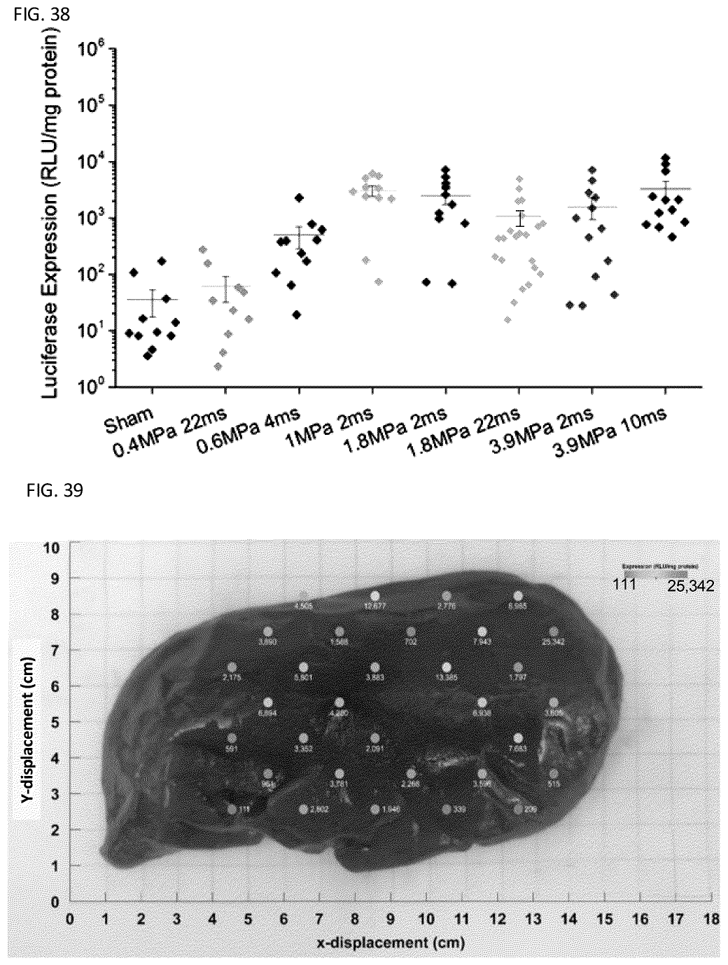

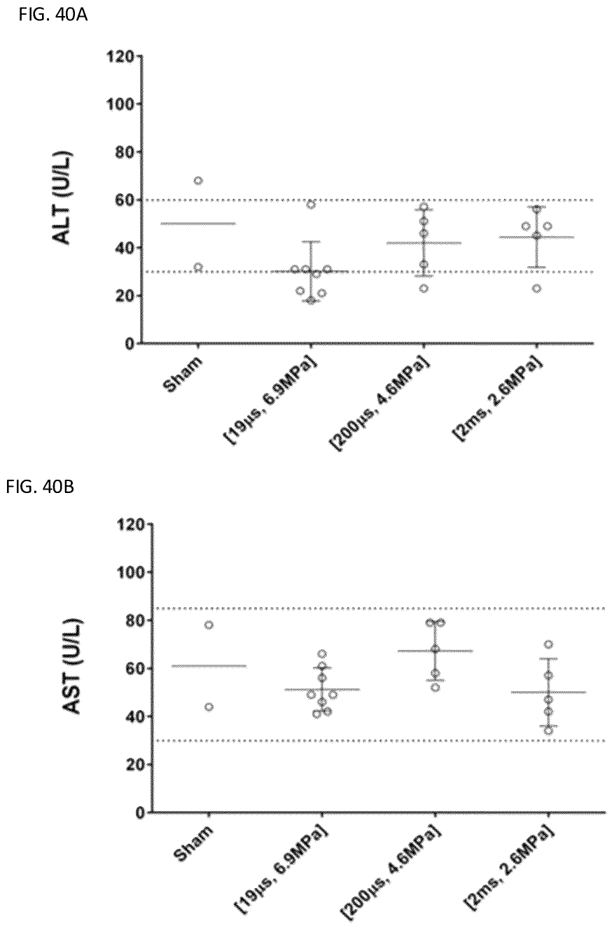

The resulting gene expression after lowering acoustic pressure and increasing PD can be found in (FIG. 37A). Three specific groups of PD-PNP parameter combinations were selected: (19 μs, 6.9 MPa), (200 μs, 4.6 MPa) and (2 ms, 2.6 MPa). The three prior mentioned US protocols were chosen based on optimal gene transfer comparison to other US protocols. Results of conditions that were tested preliminarily but not selected for repeated experiments can be found in FIG. 38. The (19 μs, 6.9 MPa) combination was used as a positive control. Using a parameter pairing of (19 μs, 6.9 MPa), a transgene expression of approximately 3500 RLU/mg protein averaged across the area of the treated lobe was obtained. Using the other two conditions, (200 μs, 4.6 MPa) and (2 ms, 2.6 MPa), an expression of approximately 3000 and 2000 RLU/mg protein was achieved, respectively. Despite differences in absolute transgene expression, there was no statistically significant difference in average expression between the parameter settings (19 μs, 6.9 MPa) and (200 μs, 4.6 MPa), or (19 μs, 6.9 MPa) and (2 ms, 2.6 MPa). The average luciferase gene expression from each US parameter group was normalized relative to the average sham-treated luciferase expression and plotted in (FIG. 37B). There is a slight decrease in gene enhancement as pressure is decreased and PD is prolonged, but all groups are significantly increased compared to sham-treated swine. Representative spatial distribution of expression values is shown in FIG. 39.

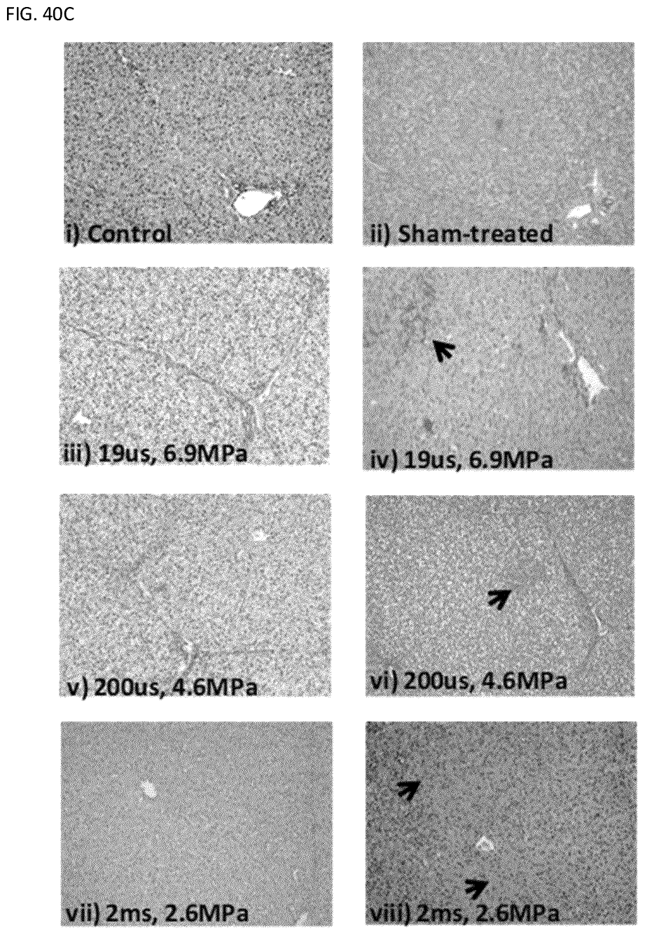

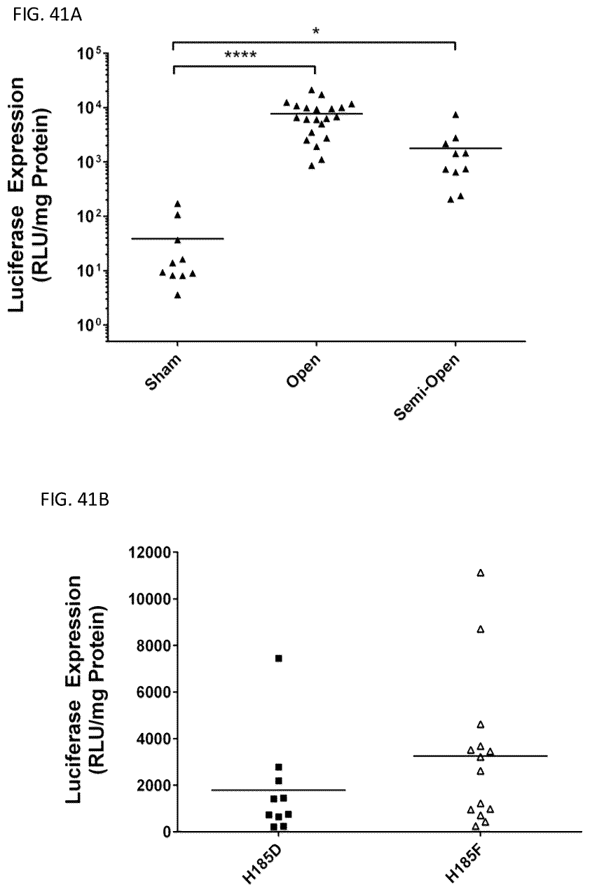

Minor tissue damage revealed in histological and transaminase analysis: Alanine aminotransaminase (ALT) (FIG. 40A) and aspartate aminotransaminase (AST) (FIG. 40B) liver enzyme levels were assessed to determine the impact of various PNP settings on liver damage. ALT and AST levels for all US-treated groups showed no significant difference from sham-treated pigs and were within the normal range. Since treated part of the liver was only, minor damage may not be detected in transaminase levels. Therefore, liver biopsies were performed 24 hours following surgery for a detailed histological examination of any potential hepatic injury.

Representative hematoxylin and eosin-stained slides of sham-treated and US-treated pigs are shown in (FIG. 40C). Control and sham-treated liver lobes (FIG. 40i and ii), wherein no pDNA/MBs infused or US applied, were used as a comparison to treated liver lobes. The portal tracts were intact, and no significant capsular inflammation was detected. The hepatic sinusoids and lobular areas were also preserved without inflammation or necrosis, and the central veins were focally distended and patent. For the majority of sections of US-treated livers, the liver parenchyma was preserved without significant tissue injury or inflammation (FIG. 40Ciii, v, vii). However, in small number of sections from all three ultrasound conditions there was focal hepatic injury including hemorrhage, congestion and rare apoptotic hepatocytes. The tissue injury was located in the pericentral region suggesting it may be related to the transient outflow obstruction (FIG. 40Civ, vi, viii). The extent of injury was minimal, accounting for less than 2% of the examined tissue from a given sample. Since the sections were harvested within 24 hours of the treatment there was no significant inflammation or repair associated with the injury.

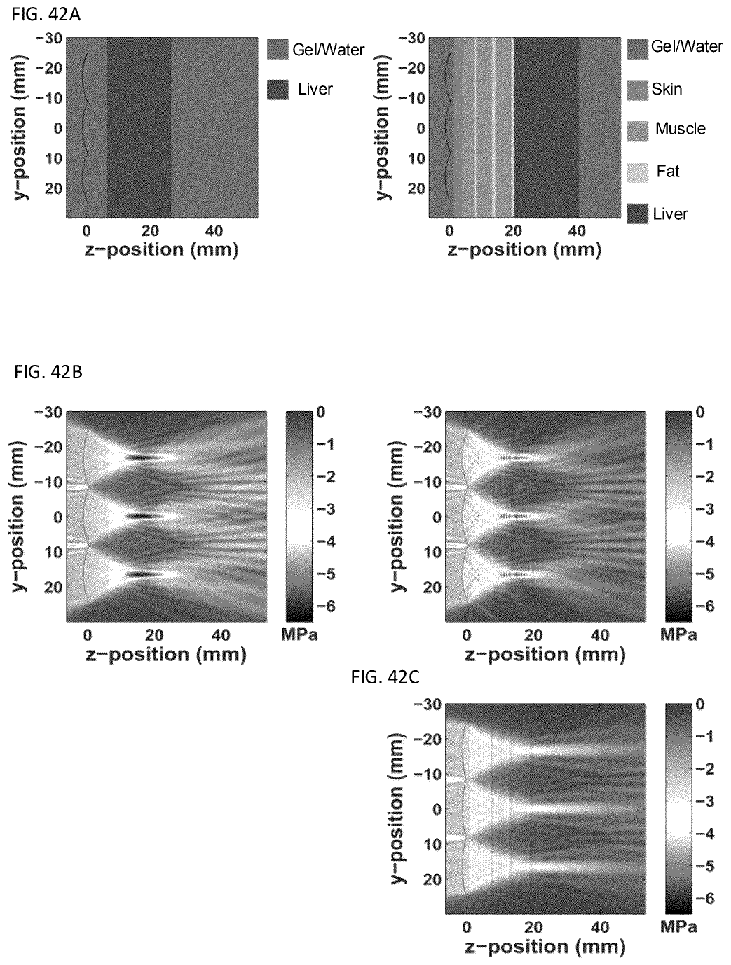

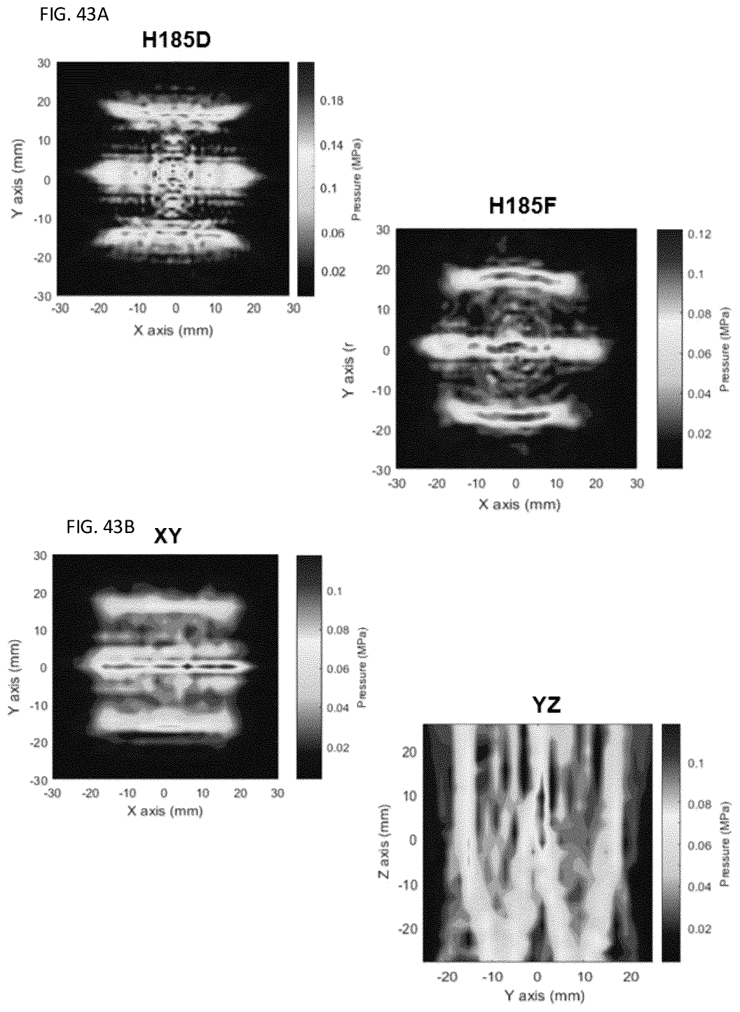

Significant gene transfer occurs in US treatment across skin: Prior results indicate UMGD can be performed using lower pressures, allowing flexibility in transducer design. The next milestone to achieve is to move towards a minimally invasive procedure for UMGD. A consideration is to perform UMGD transcutaneously. To simulate the effects of US treatment transcutaneously using UMGD, the folds of the pig skin were held together by forceps at the site of incision. The pGL4/MB solution was injected via the branch of the portal vein to the target lobe and the IVC occluded as prior. US was then administered across the skin of the pig covering the target lobe. The results of the semi-open compared to the open UMGD procedure can be seen in (FIG. 41A). Representative assayed tissue sections are selected and pooled from two pigs for each paired US parameter group. UMGD was performed using parameter settings of (19 μs, 6.9 MPa). UMGD produced significant gene transfer compared to sham both in direct contact with the target tissue, and transcutaneously across several tissue layers to the target treatment site. When treated across the skin using the same transducer in a semi-open surgical procedure, however, transgene expression is noticeably decreased. To better understand the effects of transcutaneous UMGD, a computational model was developed using the k-Wave Toolbox within MATLAB. The wave-propagation models simulate the resulting acoustic output from using H185D in a laparotomy procedure compared to a transcutaneous procedure (FIG. 42A & 42B). It was determined that the focal depth of H185D where peak US intensity occurs could not reach beyond the muscle and fatty tissue layers.

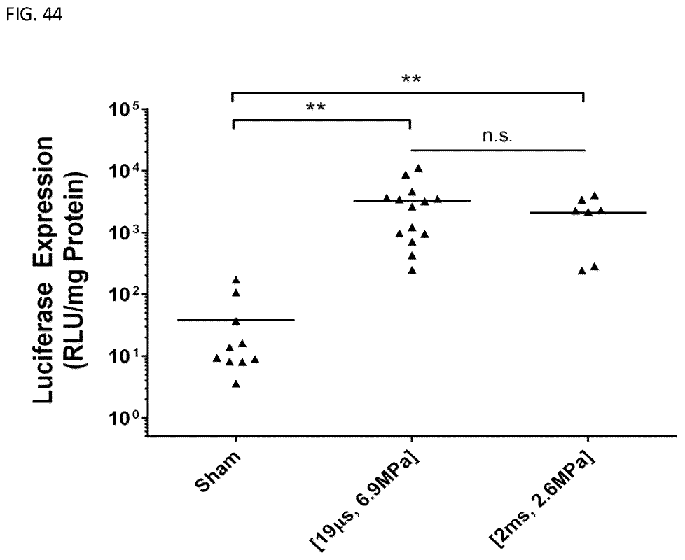

Based on this finding, the H185D transducer was revised to produce H185F with a deeper focus of 30 mm from the exit plane and a focal pressure gain of 2.79. This deeper focus and wider transition region allow H185F to align its focal region with the target liver tissue during transcutaneous treatment and achieve uniform pressures across the entire liver depth (FIG. 42C). Resulting pressure maps visualizing focal depth revision can be seen in (FIG. 43A-43B). H185F was used to perform transcutaneous UMGD in a semi-open procedure and the results can be seen in (FIG. 41B). There was a slight increase in absolute luciferase expression using H185F compared to H185D using a paired US parameter setting of (19 μs, 6.9 MPa). Transgene expression from transcutaneous UMGD using H185F was significantly greater compared to sham-treated, however. A longer pulse, low acoustic pressure condition, (2 ms, 2.6 MPa), was also tested in a semi-open procedure using H185F(FIG. 44). Luciferase expression from selected tissue of the longer PD condition was also significantly greater than sham-treated. There was no significant difference found between expressing tissue treated with the shorter PD, higher pressure and tissue treated with the longer PD, lower pressure condition.

### DISCUSSION

UMGD facilitated by MB cavitation presents a feasible and promising nonviral gene delivery strategy for clinical use to treat genetic diseases such as hemophilia. It has been shown success in developing an US/MB mediated reporter gene delivery method in murine models (Song et al., Gene Ther, 18:1006-1014, 2011; Song et al., Mol Pharm, 9:2187-2196, 2012; Shen et al., Gene Ther, 15:1147-1155, 2008) and have now scaled up into large animal models. Current surgical method of performing gene transfer requires access to the liver via laparotomy. While this procedure has been optimized for efficient gene transfer, the method of delivery is non-ideal for hemophiliac patients. Transcutaneous UMGD is a tangible solution but poses an obstacle to efficient gene transfer due to acoustic energy attenuation across the intervening tissue. While a more tightly focused transducer design (e.g. spherically focused) could potentially maintain PNP in the treatment region, the treated area would then decrease and limit total gene expression. Because of the maximum power density of the piezoceramic, there is an inherent compromise between pressure and treatment volume which is exacerbated by transcutaneous attenuation. The goal then is two-fold-to improve gene transfer while minimizing cell damage and to design a transducer to facilitate development of a minimally invasive procedure for clinical translation.

The therapeutic transducer design had previously been modified to not only significantly increase gene transfer in a porcine model, but also increase the acoustic pressure output (Noble-Vranish et al., Mol Ther Methods Clin Dev, 10:179-188, 2018). This improvement would allow us to overcome the energy attenuation barrier when treating transcutaneously. One drawback to increasing the acoustic pressure is the potential for adverse bioeffects to occur at the site of US exposure. In agreement with others (Apfel et al., Ultrasound Med Biol, 17:179-185, 1991; Holland et al., Ultrasound Med Biol, 22:917-925, 1996), it has been shown in murine models that prolonging PD can lower the acoustic pressure threshold for MB cavitation. The result of prolonging PD produced equivalent gene expression to using high pressure settings (Tran et al., J Control Release, 279:345-354, 2018). Therefore, adverse bioeffects such as severe hemorrhaging or cell death may be avoided. The intent was to scale up the method of using prolonged PD at lower acoustic pressure to a large animal model. For each experimental group the PD was increased by ten-fold, while the acoustic pressure was decreased by about 2 MPa.

These results suggest that prolonging PD and lowering acoustic pressure can produce similar results as using a high pressure, short PD setting. This finding validates the previous discovery in small animal models. This is the first instance this phenomenon has been shown in a large animal model. Several advantages exist to using longer pulse, lower power settings for MB cavitation, especially for transcutaneous treatment. One such reason is to allow transducer designs to retain as large of a treatment volume as possible at the focus without requiring an intractably large housing. Another is to increase the effective focal volume of treatment by allowing MBs to cavitate further downstream from the site of injection via microstreaming and constant blood flow (Mannaris & Averkiou, Ultrasound Med Biol, 38:681-691,2012). While the viscosity of blood may serve to dampen the radial oscillation and inertial cavitation threshold of MBs (Helfield et al., J Acoust Soc Am, 139:204-214, 2016; Helfield et al., Ultrasound Med Biol, 42:782-794, 2016), at high enough PNPs MBs may destruct upon immediate US exposure, effectively reducing treatment volume (Choi & Coussios, J Acoust Soc Am, 132:3538-3549, 2012; Skyba et al., Circulation, 98:290-293, 1998). Immediate destruction of MBs at the injection site, using high PNP conditions, has been compensated for by scanning the US transducer during treatment. However, lower PNPs may also allow cavitating MBs to persist, improving MB distribution within the vasculature and intercellular space while enhancing the volume of potential treatment (Choi & Coussios, J Acoust Soc Am, 132:3538-3549, 2012; Chen, et al., Ultrasound Med Biol, 42:528-538, 2016).

In previously published studies with the improved therapeutic transducer, H185D, it was found that for a PD of 19 μs, a PNP of at least 2.7 MPa is required for efficient gene transfer in porcine model (Noble-Vranish et al., Mol Ther Methods Clin Dev, 10:179-188, 2018). Using a PNP of 6.9 MPa resulted in even greater gene transfer with minimal tissue damage, and was therefore used as the positive control in this study. Efficient gene expression has been achieved using the long PD, lower pressure conditions tested; however, careful selection is required when using longer PDs as thermal effects may occur if too high a corresponding PNP is used. In order to validate the specific conditions tested in these experiments, a 2D Pennes' Bioheat Equation model in k-Wave was constructed and found that the maximum pressure used at each pulse duration had a CEM43 thermal dose of 0 across the transducer field. This result predicted no thermal damage from these conditions when treated for four minutes. Potential thermal effects have additionally been minimized at the transducer surface by ensuring the transducer is surrounded by degassed coupling gel during treatment. It is possible that higher PNP values are required in pigs relative to humans and other models, considering that pigs have a greater concentration of connective tissue in the liver lobule. Because MB oscillation and cavitation potential depend on the mechanical characteristics of their surrounding environment, this differing tissue composition may be responsible for the increased PNP burden observed in the porcine model (Maxwell et al., Ultrasound Med Biol, 39:449-465, 2013). Therefore, more violent MB oscillations induced by high PNPs may be needed to surpass both the lumen and layer of connective tissue to reach the target cell in swine. In comparison, pressure titration of UMGD in dogs showed that higher PNPs above 2.7 MPa had led to greater tissue damage and reduced gene expression (Noble et al., Mol Ther, 21:1687-1694, 2013). Therefore, high PNPs used in these pig studies may not be needed for efficient gene delivery in other large animals or humans, which may further lower the risk of thermal- or cavitation-associated tissue damage in those models.

Furthermore, long PDs at high PNPs can potentially induce prolonged inertial cavitation, increasing mechanical force and energy deposition (Chen, et al., Ultrasound Med Biol, 42:528-538, 2016; Pacella et al., Ultrasound Med Biol, 41:456-464, 2015; Vykhodtseva et al., Ultrasound Med Biol, 20:987-1000, 1994), resulting in significant damage to the vascular wall and cellular membranes (Hu et al., Ultrasound Med Biol, 39:2393-2405, 2013). However, this does not seem to be the case for the tested conditions by combining longer PDs with low PNPs in this study. All treated pigs showed minor damages in limited areas of their livers when examined by detailed histological analysis. Nevertheless, careful considerations should be given to choose a combination of PD and PNP settings for optimal gene transfer with minimal tissue damage. It should also be noted that in a previous rat study (Song et al., Mol Pharm, 9:2187-2196, 2012), damage in the liver was repaired quickly and the liver returned to normal within a few days.

The next step was to determine whether applying lower pressure, longer PD was translatable to transcutaneous UMGD. When treating with H185D across the skin, the expression was reduced compared to directly treating the surface of the liver with US. This indicates the importance of considering the dissipation of acoustic energy across several tissue layers before reaching the surface of the liver. Transitioning from open to transcutaneous UMGD also requires considering the focal depth of the therapeutic transducer. The focus of H185D, where the largest focal gain occurs, lies at 20 mm from the exit plane as measured in degassed water using a hydrophone. Based on distances measured using diagnostic ultrasound, this would place the distance of greatest acoustic output within fat and muscle, slightly shallower than the liver surface. Simulation in k-Wave suggests that this focus in fact becomes shallower when propagating through fat and muscle tissue in the transcutaneous case, resulting in an effective focus 5-10 mm shallower than the liver surface. Beyond the effective focus, the pressure field of H185D becomes scattered, reducing the focal gain and US intensity. As this simulation suggests, this far-field effect may be enhanced by the non-linearity of acoustic wave propagation across soft-tissue (Duck, Proc Inst Mech Eng H, 224:155-170, 2010). Therefore, H185F was designed to compensate for the depth of the liver with a revised focal depth of 30 mm from the exit plane. Absolute gene expression was increased compared to treatment with H185D, but expression was still decreased when compared to results from open surgeries. This suggests that focal depth is an important design parameter in improving future transducers, but that acoustic pressure is still a determinant for sonoporation. However, when comparing a high pressure, short PD pairing to a longer pulse, lower pressure pairing using H185F, there was no significant difference in gene transfer. This suggests the possibility that use of low pressures with prolonged PDs can be translatable to transcutaneous UMGD.

### CONCLUSIONS

In conclusion, these data demonstrated that: (i) prolonging PD lowers the acoustic pressure required for efficient gene delivery in UMGD and is scalable to large animals; and (ii) focal depth is a factor to consider in transducer design for transcutaneous UMGD in addition to acoustic pressure. This success in scaling results from small to large animal models was achieved only by solving the challenges of larger treatment area/volume and overcoming multiple physical barriers to UMGD efficacy such as tissue attenuation and evolving transducer requirements. Future studies to consider include varying the number of transducer elements used, the focal depth, and other beam patterns such as coherent and incoherent phasing to increase effective treatment area. The intent of the experimental approach used was to minimize experimental variance when comparing results of transcutaneous treatment to those from previous direct-scanning experiments. The goal for the semi-open approach was to isolate the effects of the ultrasound transducer and signal parameters used. This work is significant to help us explore the optimal US conditions to be used in a less/noninvasive transcutaneous ultrasound treatment approach in large animal studies and clinical translation. Despite the present challenge of overcoming signal attenuation across multiple tissue barriers, the current study validates previous findings that longer PDs can lower the acoustic pressure threshold needed for efficient gene delivery with minimal tissue damage and shows that this result scales to large animal models. These US protocols may be used in an external application of UMGD for effective gene transfer in a minimally invasive operation. While this study does not directly evaluate a specific disease model, UMGD is a viable candidate for therapeutic gene delivery, and one of its key advantages in comparison to viral methods is its relative mechanistic invariance with respect to the size and specific sequence of the DNA to be delivered. The herein described method of UMGD targeting the liver will benefit a large number of patients with inherited or metabolic diseases, such as hemophilia, MPSI, MPSII, and many others if translated to clinical application. In addition, the liver can be used to synthesize other potential target proteins for treatment of acquired diseases such as cancer. Overall, these results have significant implication for achieving successful translation of US technology for gene delivery to the clinic.

### Example 6: Therapeutic Levels of Factor VIII Gene Expression were Achieved by

Transcutaneous Ultrasound Mediated Gene Delivery into Canine Liver

In this Example, the established transcutaneous UMGD procedure (described above) was investigated to transfer human factor VIII (hFVIII) plasmid in a canine model.

Background: Ultrasound mediated gene delivery (UMGD) combined with microbubbles (MBs) has been shown to be an effective method for non-viral gene delivery, with potential for treating genetic diseases like hemophilia A. Previous studies (see above) demonstrated successful transfection of swine liver using luciferase reporter plasmids and a transcutaneous UMGD procedure.

Methods: A high-expressing liver-specific hFVIII plasmid was injected in combination with RN18 MBs into canine liver lobes through a balloon catheter inserted into the hepatic vein via jugular vein access. Simultaneously, transcutaneous therapeutic ultrasound was applied to enhance gene transfer using an eight-minute pulsed US treatment (4.8 MPa, 1.05 MHZ, 50 Hz PRF). Blood was collected at multiple time points for four weeks. A Western blot was performed to examine the presence of hFVIII in the treated animals. ELISA was also carried out to evaluate the levels of hFVIII expression and antibody formation to hFVIII, and transaminase (AST/ALT) assays were performed to assess potential liver damage following treatment.

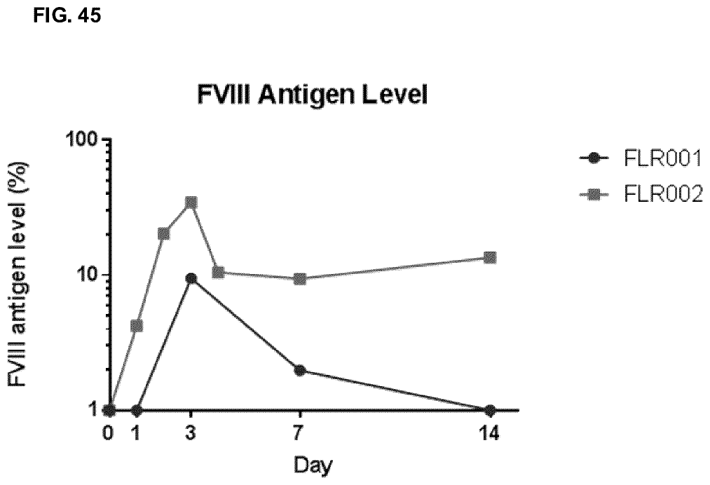

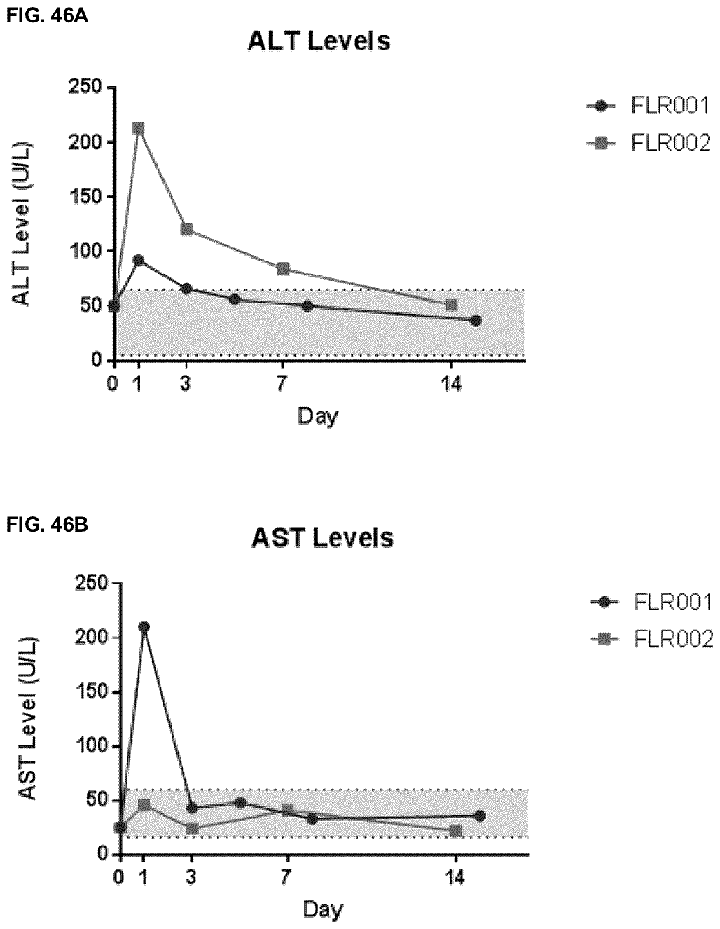

Results: The Western blot confirmed the presence of hFVIII protein in the plasma of treated dogs. hFVIII expression was detected by hFVIll-specific ELISA with levels between 9 and 35% of normal levels at day three and persisted between 1 and 10% at day seven (FIG. 45). Minor elevation in the AST/ALT levels was observed at day one, but these levels decreased to normal by day three and no other parameters from CBC and whole blood chemistry analysis showed any significant changes (FIGS. 46A and 46B).

Conclusions: Transcutaneous UMGD of hFVIII plasmids achieved successful transfection of liver tissue and produced therapeutic levels of hFVIII expression in a canine model. These results show significant promise for a minimally invasive UMGD as a clinically feasible therapy for hemophilia.

### Example 7: Transcutaneous Ultrasound Mediated Gene Delivery in Canine Liver

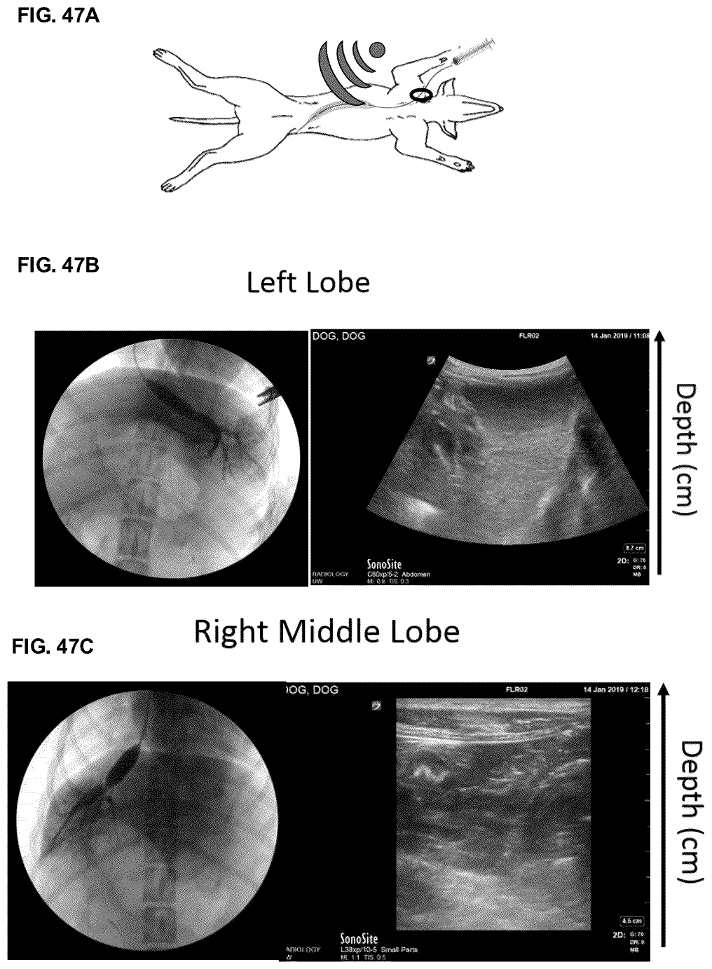

Ultrasound mediated gene delivery (UMGD) in combination with microbubbles (MBs) has been shown to be an effective method for non-viral gene delivery. UMGD is an especially promising strategy for treating genetic diseases, including Hemophilia A, which causes a deficiency of functional Factor VIII and inhibits proper execution of the clotting cascade. Previous studies showed successful transfection of swine liver using luciferase reporter plasmids and a transcutaneous UMGD procedure. As described above, the established transcutaneous UMGD procedure was used to transfer human factor Vill plasmid in a canine model; FIG. 47A is a schematic illustration of this model.

A high-expressing liver-specific human factor VIII plasmid was injected in combination with RN18 MBs into the canine liver lobes through a fluoroscopy-guided balloon catheter inserted into the hepatic vein via jugular vein access. Simultaneously, transcutaneous therapeutic ultrasound was applied to induce cavitation in the liver using an eight-minute pulsed US treatment (4.8 MPa, 1.05 MHz, 50 Hz PRF). Images are shown in FIGS. 47B and 47C. Blood was collected prior to gene delivery and then at days one and three followed by weekly time points for four weeks. A Western blot was performed to examine the presence of human factor VIII in the treated animals. ELISA was also carried out to evaluate the levels of human factor VIII expression and transaminase (AST/ALT) assays were performed to assess potential liver damage at various time points following treatment.

The Western blot confirmed the presence of human factor VIII protein in the plasma of treated dogs. ELISA data confirmed notable expression of human factor VIII at approximately 9% of normal plasma levels at day three, followed by lower expression levels at day seven. The formation of species-specific inhibitor antibodies at later time points is being investigating. The AST/ALT data showed minor elevation above normal levels at day one, but these levels had completely normalized by day three. All the other parameters from CBC and whole blood chemistry analysis showed little or no changes from the normal levels over the entire experimental period. Transcutaneous UMGD of human factor VIII plasmids achieved successful transfection of liver tissue and produced therapeutic levels of human factor VIII expression in a canine model. Taken together, these results show significant promise for a minimally invasive UMGD as a clinically feasible therapy for Hemophilia.

### Example 8: Therapeutic Levels of FVIII Gene Expression were Detected Following Transcutaneous UMGD of pLP-hF8-X10 into the Dog Liver

This example describes therapeutic levels of FVIII gene expression can be achieved following transcutaneous UMGD of a liver-specific, high-expressing hFVIII-X10 plasmids. These studies indicate that non-invasive transcutaneous UMGD can be translated into clinical application to treat hemophilia A.

Four normal dogs (FLR001-FLR004) were treated with transcutaneous UMGD of hFVIII-X10 plasmids. These experiments were performed using the same transcutaneous procedure as described in Example 5 without open surgery.

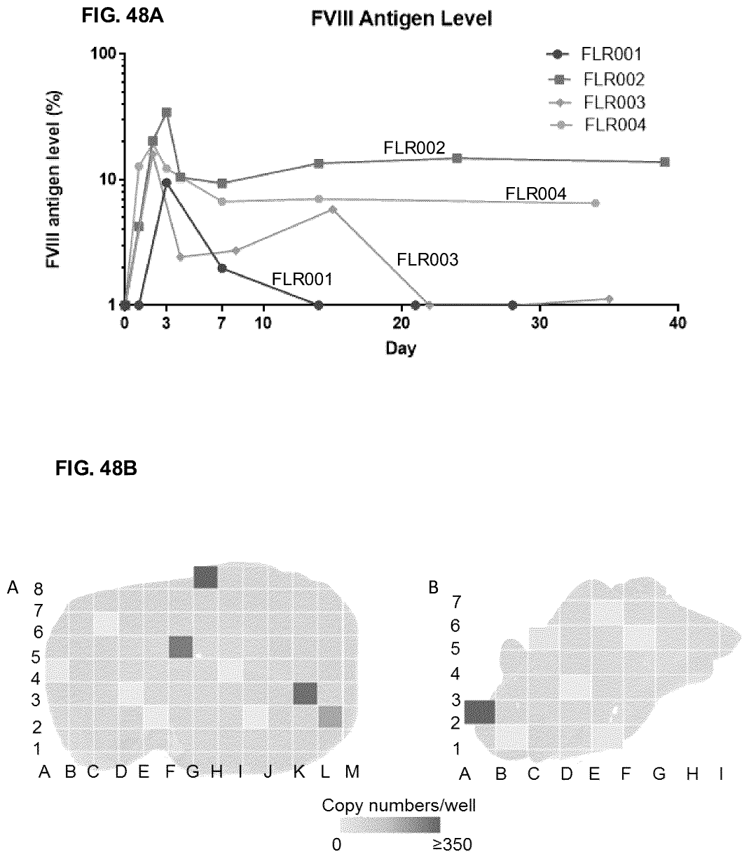

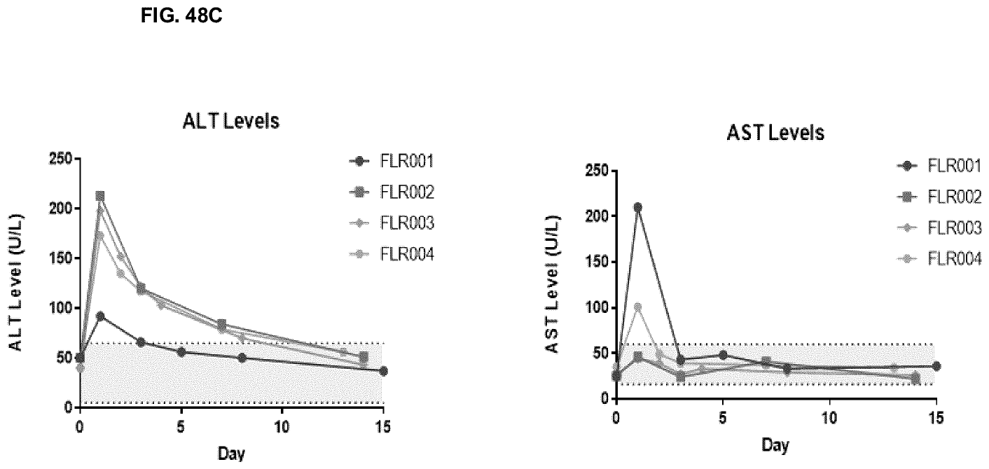

The high-expressing liver-specific pLP-hF8-X10 plasmid was injected in combination with RN18 MBs into canine liver lobes through a balloon catheter inserted into the hepatic vein via jugular vein access. Simultaneously, transcutaneous therapeutic ultrasound was applied to enhance gene transfer using an eight-minute pulsed US treatment using the focused transducer H114 (1.05 MHz, 2.5 MPa derated PNP, 200 μs PD and 50 Hz PRF). Two US treatments were carried out on separate liver lobes (right middle and left lobes, respectively) for each dog. Blood was collected at multiple time points for 4-8 weeks. HFVIII expression was detected by hFVIII-specific ELISA with levels between 9 and 35% of normal levels at day three and persisted between 1 and 10% at day seven (FIG. 48A). Both FLR002 and FLR004 retained nearly 10% of normal level through day 28 and beyond. A very low titer anti-hFVIII antibody was only detected in FLR002, which did not impact hFVIII expression, and no antibodies were detected in the other three treated dogs. This is somewhat surprising since it was hypothesized that species-specific anti-hFVIII can be induced in normal dogs. The normal dogs may be highly tolerant to hFVIII due to high homology between human and canine FVIII.

At day 30 for FLR001 and day 60 for FLR002, the treated dogs were euthanized and livers harvested. Small chunks of liver tissues were randomly selected for confirming the persistence of plasmid vector in the liver lobes. As expected, most of the transfection occurred in right middle lobe and left lobe. Representative maps in right middle and left lobe for FLR002 are shown in FIG. 48B.

Furthermore, it was found that liver enzyme ALT was somewhat elevated in all experimental dogs on day 1 following surgery; AST was also slightly elevated, but did not exceed the normal range for FLR002 or FLR003 (FIG. 8C). All ALT levels returned to normal within one week, while AST levels dropped more rapidly to normal within three days. No notable liver damage was found upon necropsy of FLR001 at 30 days post-surgery or FLR002 at 60 days post-surgery. Histology examination supports that no notable damage was detected in the liver lobes of these two treated dogs.

These data are very encouraging, suggesting that UMGD has great potential for therapeutic treatment of HemA. However, as shown in the FIG. 48A, there are variations of hFVIII levels and the persistence of hFVIII expression among the four treated normal dogs.

As will be understood by one of ordinary skill in the art, each embodiment disclosed herein can comprise, consist essentially of or consist of its particular stated element, step, ingredient, or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. As used herein, a material effect would cause a statistically-significant reduction in transient expression of a therapeutic protein within 7 days following administration of a disclosed nanocarrier to a subject.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of +20% of the stated value; +19% of the stated value; +18% of the stated value; +17% of the stated value; +16% of the stated value; +15% of the stated value; +14% of the stated value; +13% of the stated value; +12% of the stated value; +11% of the stated value; +10% of the stated value; +9% of the stated value; +8% of the stated value; +7% of the stated value; +6% of the stated value; +5% of the stated value; +4% of the stated value; +3% of the stated value; +2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference for their particular cited teachings.

It is to be understood that the embodiments disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A transcutaneous ultrasound-mediated delivery method for administering a therapeutic compound to a target tissue in a subject, the method comprising:
b. positioning a balloon catheter in a blood vessel of the subject such that the balloon is adjacent to the target tissue;
c. inflating the balloon catheter to occlude outflow from a region adjacent to the target tissue;
d. administering the therapeutic compound to the vessel of the subject such that it is substantially retained adjacent to the target tissue by the balloon catheter;
e. determining the location of the therapeutic compound and/or a detectable adjunct compound administered therewith using at least one of diagnostic ultrasound, radiography, or fluorography; and
f. administering therapeutic ultrasound energy (sonication) transcutaneously, such that the energy mediates delivery of the therapeutic compound across the vessel wall and into the adjacent target tissue.

2. The method of claim 1, further comprising administering a composition comprising a coagulation factor to the subject before the therapeutic ultrasound energy (sonication) is administered.

3. The method of claim 1, wherein the therapeutic compound and/or the adjunct compound comprises microbubbles (MBs).

4. The method of claim 1, wherein the therapeutic compound comprises a nucleic acid molecule capable of expression in at least one cell type in the target tissue.

5. The method of claim 4, wherein the nucleic acid molecule comprises naked plasmid DNA encoding at least one peptide, protein, or functional RNA molecule.

6. The method of claim 1, wherein the detectable adjunct compound comprises an ultrasound contrast agent.

7. The method of claim 1, wherein the transcutaneous therapeutic sonication is performed using parameter pairings between the 1 and 3 J energy curves.

8. The method of claim 1, wherein the transcutaneous therapeutic sonication is performed using parameter pairings that can generate effective energy for efficient MB cavitation and gene transfer, as described.

9. The method of claim 1, wherein the transcutaneous therapeutic sonication is performed using an ultrasound transducer selected from model H114, XDR106-5E, and XDR106-10E.

10. The method of claim 1, wherein the transcutaneous therapeutic sonication is performed with a frequency of about 0.5-3 MHZ.

11. The method of claim 1, wherein the transcutaneous therapeutic sonication is performed for a period of between about 10 seconds and about 15 minutes.

12. The method of claim 1, wherein the target tissue is at least 1 cm below, at least 2 cm below, at least 3 cm below, at least 4 cm below, at least 5 cm below, or more than 5 cm below the dermis of the subject.

13. The method of claim 1, wherein the targeted tissue is a liver tissue.

14. The method of claim 1, wherein the targeted tissue is a tumor tissue.

15. The method of claim 14, wherein the tumor tissue is a brain tumor tissue, ovarian tumor tissue, breast tumor tissue, liver tumor tissue, kidney tumor tissue, head tumor tissue, neck tumor tissue, colon tumor tissue, or a combination thereof.

16. A transcutaneous ultrasound-mediated drug delivery system for use with the method of any one of claims 1-15, the system comprising: an ultrasound apparatus, a therapeutic compound capable of being administered to the target tissue, and a balloon catheter.

17. The system according to claim 16, wherein the ultrasound apparatus comprises: a function generator for generating the sonication; an amplifier connected with the function generator to amplify the sonication; a power meter connected with the amplifier; and a transducer connected between the power meter and a removable surface for transferring the sonication to therapeutic compound adjacent to the target tissue, wherein the transducer is selected from model H114, XDR106-5E, and XDR106-10E.

18. The system according to claim 16, further comprising an ultrasound contrast agent administered before or in conjunction with administration of the therapeutic compound, wherein the ultrasound contrast agent comprises microbubbles.

19. The system according to claim 16, further comprising a diagnostic ultrasound device, radiography device, or fluorography device for determining the location of the therapeutic compound and/or a detectable adjunct compound administered therewith.

20. The system of claim 16, wherein the targeted tissue is liver tissue.

21. The system of claim 16, wherein the targeted tissue is a tumor tissue.

22. The system of claim 21, wherein the tumor tissue is brain tumor tissue, ovarian tumor tissue, breast tumor tissue, liver tumor tissue, kidney tumor tissue, head tumor tissue, neck tumor tissue, colon tumor tissue, or a combination thereof.

23. A method for transcutaneous ultrasound treatment to tissue internal to a subject, the method comprising: obtaining percutaneous access to a target site within the subject, percutaneous delivery and capture of administered therapeutic compound(s) adjacent to a tissue to be treated at the target site, fluoroscopic-assisted and/or diagnostic ultrasound-assisted targeting of the therapeutic compound(s), and microbubble insonation at the target site using transcutaneously ultrasound, thereby releasing the therapeutic compound(s) into tissue at the target site.

24. The method of claim 23, wherein the transcutaneous ultrasound insonation is applied using a model H114, XDR106-5E, or XDR106-10 ultrasound transducer.


SEQUENCE LISTING (2 sequences)

SEQ ID NO: 1 - DNA, 22 bp, synthetic construct
  misc_feature 1..22: note = synthetic oliogonucleotide
  source 1..22: mol_type = other DNA
ccagagttccaagcctccaaca

SEQ ID NO: 2 - DNA, 23 bp, synthetic construct
  misc_feature 1..23: note = synthetic oligonucleotide
  source 1..23: mol_type = other DNA
ggaagtcagtctgtgctccaatg